SEQUENCE LISTING (354 sequences)

SEQ ID NO: 1 - DNA, 90 bp, Artificial sequence
  Synthetic polynucleotide
tatacacttctgcttaggatgataattggaggcaagtgaatcctgagcgtgatttgataatgacctaataatgatgggttttatttccag

SEQ ID NO: 2 - DNA, 22 bp, Artificial sequence
  Synthetic polynucleotide
acccagcctgacaccaaattta

SEQ ID NO: 3 - DNA, 22 bp, Artificial sequence
  Synthetic polynucleotide
tactaaaaggcagcctcctaga

SEQ ID NO: 4 - DNA, 22 bp, Artificial sequence
  Synthetic polynucleotide
attggctaccttggttggatga

SEQ ID NO: 5 - DNA, 22 bp, Artificial sequence
  Synthetic polynucleotide
gacagctggctatccaggattc

SEQ ID NO: 6 - DNA, 22 bp, Artificial sequence
  Synthetic polynucleotide
acttgcaggaggtgagggatta

SEQ ID NO: 7 - DNA, 22 bp, Artificial sequence
  Synthetic polynucleotide
attagggaatgcagactctggg

SEQ ID NO: 8 - DNA, 22 bp, Artificial sequence
  Synthetic polynucleotide
tgggtgagattagaggccactg

SEQ ID NO: 9 - DNA, 22 bp, Artificial sequence
  Synthetic polynucleotide
tgcttcctcccttgtctcccta

SEQ ID NO: 10 - DNA, 22 bp, Artificial sequence
  Synthetic polynucleotide
tggcatatgagaaaagtcacag

SEQ ID NO: 11 - DNA, 22 bp, Artificial sequence
  Synthetic polynucleotide
ccttattcttttgatatactcc

SEQ ID NO: 12 - RNA, 80 nt, Artificial sequence
  Synthetic polynucleotide
guuuuaguacucuggaaacagaaucuacuaaaacaaggcaaaaugccguguuuaucucgucaacuuguuggcgagauuuu

SEQ ID NO: 13 - DNA, 20 bp, Artificial sequence
  Synthetic polynucleotide
agagcaacagtgctgtggcc

SEQ ID NO: 14 - RNA, 20 nt, Artificial sequence
  Synthetic polynucleotide
agagcaacagugcuguggcc

SEQ ID NO: 15 - DNA, 22 bp, Artificial sequence
  Synthetic polynucleotide
acccagcctgacaccaaattta

SEQ ID NO: 16 - DNA, 22 bp, Artificial sequence
  Synthetic polynucleotide
tactaaaaggcagcctcctaga

SEQ ID NO: 17 - DNA, 22 bp, Artificial sequence
  Synthetic polynucleotide
attggctaccttggttggatga

SEQ ID NO: 18 - DNA, 22 bp, Artificial sequence
  Synthetic polynucleotide
gacagctggctatccaggattc

SEQ ID NO: 19 - DNA, 22 bp, Artificial sequence
  Synthetic polynucleotide
acttgcaggaggtgagggatta

SEQ ID NO: 20 - DNA, 22 bp, Artificial sequence
  Synthetic polynucleotide
attagggaatgcagactctggg

SEQ ID NO: 21 - DNA, 22 bp, Artificial sequence
  Synthetic polynucleotide
tgggtgagattagaggccactg

SEQ ID NO: 22 - DNA, 22 bp, Artificial sequence
  Synthetic polynucleotide
tgcttcctcccttgtctcccta

SEQ ID NO: 23 - DNA, 22 bp, Artificial sequence
  Synthetic polynucleotide
tggcatatgagaaaagtcacag

SEQ ID NO: 24 - DNA, 22 bp, Artificial sequence
  Synthetic polynucleotide
ccttattcttttgatatactcc

SEQ ID NO: 25 - RNA, 80 nt, Artificial sequence; identical to SEQ ID NO: 12
  Synthetic polynucleotide

SEQ ID NO: 26 - DNA, 102 bp, Artificial sequence
  Synthetic polynucleotide
acccagcctgacaccaaatttaguuuuaguacucuggaaacagaaucuacuaaaacaaggcaaaaugccguguuuaucucgucaacuuguuggcgagauuuu

SEQ ID NO: 27 - DNA, 102 bp, Artificial sequence
  Synthetic polynucleotide
tactaaaaggcagcctcctagaguuuuaguacucuggaaacagaaucuacuaaaacaaggcaaaaugccguguuuaucucgucaacuuguuggcgagauuuu

SEQ ID NO: 28 - DNA, 102 bp, Artificial sequence
  Synthetic polynucleotide
attggctaccttggttggatgaguuuuaguacucuggaaacagaaucuacuaaaacaaggcaaaaugccguguuuaucucgucaacuuguuggcgagauuuu

SEQ ID NO: 29 - DNA, 102 bp, Artificial sequence
  Synthetic polynucleotide
gacagctggctatccaggattcguuuuaguacucuggaaacagaaucuacuaaaacaaggcaaaaugccguguuuaucucgucaacuuguuggcgagauuuu

SEQ ID NO: 30 - DNA, 102 bp, Artificial sequence
  Synthetic polynucleotide
acttgcaggaggtgagggattaguuuuaguacucuggaaacagaaucuacuaaaacaaggcaaaaugccguguuuaucucgucaacuuguuggcgagauuuu

SEQ ID NO: 31 - DNA, 102 bp, Artificial sequence
  Synthetic polynucleotide
attagggaatgcagactctgggguuuuaguacucuggaaacagaaucuacuaaaacaaggcaaaaugccguguuuaucucgucaacuuguuggcgagauuuu

SEQ ID NO: 32 - DNA, 102 bp, Artificial sequence
  Synthetic polynucleotide
tgggtgagattagaggccactgguuuuaguacucuggaaacagaaucuacuaaaacaaggcaaaaugccguguuuaucucgucaacuuguuggcgagauuuu

SEQ ID NO: 33 - DNA, 102 bp, Artificial sequence
  Synthetic polynucleotide
tgcttcctcccttgtctccctaguuuuaguacucuggaaacagaaucuacuaaaacaaggcaaaaugccguguuuaucucgucaacuuguuggcgagauuuu

SEQ ID NO: 34 - DNA, 102 bp, Artificial sequence
  Synthetic polynucleotide
tggcatatgagaaaagtcacagguuuuaguacucuggaaacagaaucuacuaaaacaaggcaaaaugccguguuuaucucgucaacuuguuggcgagauuuu

SEQ ID NO: 35 - DNA, 102 bp, Artificial sequence
  Synthetic polynucleotide
ccttattcttttgatatactccguuuuaguacucuggaaacagaaucuacuaaaacaaggcaaaaugccguguuuaucucgucaacuuguuggcgagauuuu

SEQ ID NO: 36 - protein, 1012 aa, Artificial sequence
  Synthetic polypeptide
MVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 37 - DNA, 90 bp, Artificial sequence; identical to SEQ ID NO: 1
  Synthetic polynucleotide

SEQ ID NO: 38 - DNA, 10640 bp, Artificial sequence
  Synthetic polynucleotide
gtagttcttttgttcttcactattaagaacttaatttggtgtccatgtctctttttttttctagtttgtagtgctggaaggtatttttggagaaattcttacatgagcattaggagaatgtatgggtgtagtgtcttgtataatagaaattgttccactgataatttactctagttttttatttcctcatattattttcagtggctttttcttccacatctttatattttgcaccacattcaacactgtatcttgcacatggcgagcattcaataactttattgaataaacaaatcatccattttatccattcttaaccagaacagacattttttcagagctggtccaggaaaatcatgacttacattttgccttagtaaccacataaacaaaaggtctccatttttgttaacattacaattttcagaatagatttagatttgcttatgatatattataaggaaaaattatttagtgggatagttttttgaggaaatacataggaatgttaatttattcagtggtcatcctcttctccatatcccaccctaagaacaacttaacctggcatatttggagatacatctgaaaaaatagtagattagaaagaaaaaacagcaaaaggaccaaaactttattgtcaggagaagactttgtagtgatcttcaagaatataacccattgtgtagataatggtaaaaacttgctctcttttaactattgaggaaataaatttaaagacatgaaagaatcaaattagagatgagaaagagctttctagtattagaatgggctaaagggcaataggtatttgcttcagaagtctataaaatggttccttgttcccatttgattgtcattttagctgtggtactttgtagaaatgtgagaaaaagtttagtggtctcttgaagcttttcaaaatactttctagaattataccgaataatctaagacaaacagaaaaagaaagagaggaaggaagaaagaaggaaatgaggaagaaaggaagtaggaggaaggaaggaaggaaagaaggaaggaagtaagagggaagcagtgctgctgctgtaggtaaaaatgttaatgaaaatagaaattaagaaagactcctgaaaggcaattatttatcaatatctaagatgaggagaaccatattttgaagaattgaatatgagacttgggaaacaaaatgccacaaaaaatttccactcaataaatttggtgtcaggctgggtgcagtggctcacacttgtaatcctagcacttttggaggcagaggcaggtgaattgcttgagtccaggagtttgagaccagcgtgggcaacatggcaaaccccacctctacaaaaaacacaaacaaaagaaaatagctgggtgtggtggtgtgtgcctgtagtcccagctacttgggaggctgaggtgggaggatcacctgagcctgagaagtggaggctgcagtgagccatgattgcaccactgtaccctagcctaggtgataggctcaaaaaaaaaaaaaattggtgtttgcaatgctaataatacaatttggttgtttctctctccagttgttttcctacatacgaaacagcttttaaaacaaaatagctggaattgtgcattttttcttacaaaaacattttctttcttaaaatgttattatttttcttttatatcttgtatattattactagcagtgttcactattaaaaaattatactataggaggggctgatactaaataagttagcaatggtctaaacaaggatgtttatttatgaaaaggtagtaattgtgtttcatagaatttttaaaattaattctgcgtatgtcttcaagatcaattctatgatagatgtgcaaaaatagctttggaattacaaattccaagacttactggcaattaaatttcaggcagttttattaaaattgatgagcagataattactggctgacagtgcagttatagcttatgaaaagcagctatgaaggcagagttagaggaaggcagtggtcccttgggaatatttaaacacttctgagaaacggagtttactaactcaatctaggaggctgccttttagtagtattaggaatggaacactttatagttttttttggacaaaagatctagctaaaatataagattgaataattgaaaatattaacattttaagttaaatcttacccactcaatacaatttggtaatttgtatcagaagcttaaaagataacctaatagttcttctacttctataacttacccaaatatgtttgcagagatcttatgtaaagctcttcattataacactgctttcaggagccaaaaattgggtgggggagccccataaatgttgaataataggggtttgattagataaattttggtgtagttctataatggcgtgttattcagccaataaaaggtttgttaaagaatgactgtgacggatgtatatgatatactcttaagtgaataaagagttacaaaatgttatgtacaagttacaaaatgtatgtacattatgatccatttttcataaaatcatatgtatgtatatatgtgtgtctggaaggataaatttatcaagttgttatctctgaaattttgggtatattttatatttctagattttctgttactttgttactttactgataaagtaataacgttgttgacttttgtcactctcccctattaataatcatctaggctgcaaaaggatcatgtcttctttatttttatattccaaggactgtcaacaagtgcctagcacttgacaggtatattatagaaatttaactgaatatctttaggaaatagatttttgtttgtagttgttctagtctacattaaatgtcttgcgcttatgaaacttccttgaattattttagtgaagcaatattagtatagaattttgcatcactggatgcccttgactgaaagctggcttatggcatctcaccagtgtgtggggagtttcagtccttctgttgtctgcatcacagctgaagcagtgctgttgctgacaattcctgacaccaccttgtctctattattgatcattgcctcactatggtactgagttttagcttattcttgtaataactgggactcatatgtatagaataagctattagctcacgtttttgcttgctttttatacagaatacatgtctgcaaatagttttatcaatattttggaattttgggagatatgaagttaaaaacatcattgaatatatatatatacacacacacatatatatatgacactatacatgatttattttatttaatttttaaaattttattctttttagagattaggtcttactctgtcacccaggctgaacttcagtggtgtgatcatagctcactgtaaccttgaactcctgggctcaattgacctttccgcttcagcctcccaaagtgctgggtttataggcatgagccactgtgtctggtccaatatgcatatatatatttttaacctggattatcagagctatattgtgtttaggtttataaagctgtactatgtgaaaatatcacttctaggtttaattttgtacaaaggaattttatatagaaatgaggtaattcagattttttcccatgtaataagaattgtaaaatttactgaaacaaacatcaaaaagatatctgttacatgaccttcctttcttttgaatatatttcaggtgatattatttattaaaatttaaaaatgaaaattaaaatatataaaaagttgaaaattattcctttctttactgtctctcatctgtccattttccattctcctgcattccctcatccaaccaaggtagccaatccaggtaactttttttagtatcttcccagagatgtttctctctatatatataatcaatatacattttttattattccccacctctctttttatgtaacaatatgcagagttttgcttcttgcttttcccactatcttggacaactttccatattcaaagcacagaggacttgcacatatgttcagactgctgaatatttctgtctctcccctgccattcatatgttgaaatcctaattcccaaggtgatggtattgcagggtggggcctttgggaggtgattagtccatgagggtgaagtctttagtaaatgagattagtgtctttataaaagaaaccttagagagaccctcacaccttagagagaccctcacccctttctgccatgtgagaacacagcaggaagacagctggctatccaggattcaggagtctcttagcagacccaaatctgctggcaccttgatcttggacttcccagcctccagaactgtgagaaataaattcctgttgtttataagccacacagttcatggtattttgttatagcagcctgaacaaggacacacacacacacacacacacatgcacacacatttaaatagatgcatagtattctatcatatggatggatattctatgatataatgaatcactattgattgacatttgggttgtttccaatattttgttaacacaaagaacaacactacaaataactttatatacatatcatttagcacatctgcaattgtatcagtaggcttcctataagtggtcaagcatttgtgtacttgtgattttggtagatgttgtcaaatgtccttccctgaaatttgtaccaattcgtactcatgccatacactctaaatagagtgctgatttccccacagcattactaacagatgatattatctaatttaaaaagtttctcatcttatagggaaaatagtatgtcaatgtattcttaacttgcatttcttttattataagtagtgtaaaatatcatttcaacttatacacaggaggaatttctctctatataaagtgatcctagaatcataatgaaaaatatcaccaactcattaggaaaatgtacaaaggattgaatagatatctcatcaaaaataaaaatataagtggcctttaaacattgaaaggtaacatttgaacaaagacttgcaggaggtgagggattagggaatgcagactctgggaagagtcttccaagtagcaggtgaagcaagtgcaaagctttcagatgggactgactatacctgtctggtttgaagaacagtaaggaggtcactgaggctggcatagagtaagacagggagggtagaatactgtcagagaagtaatcggcggtggaggtagggggtaaaccataaagtgctcgtaaagactaaggcttatttctctgggtgagattagaggccactggagagttttaaacagaagtaacagggccactttggctaatgtttttaggctattctgtagggagacaagggaggaagcaaggagatgagttaggagtctattgtgccagttcaggcaagtgatgatggtggcttgatccaggtagtagtggaagtagtatagtaggaagtgatcagattcaggacatgctttgaaggaagatccaataggattaatggataagttgaacaatggcatatgagaaaagtcacagaggagtcaaagatgattccaagctttctggactgagtaactggaaggataaatgtgccgtttactagaaagataatgggagaaacaggttttggatggagcttggtttgggaatattaagtttgaaatgcctatttgacatccaaatagagatgttagttggatgtacaagtctagtttcaaggaagagggggctggtagtgtgaagatggggctggataagattctaaaggaaagagggttgataagaagagaaaggggtgtaggggttagcctaagggcattctaagtattagaggttaaggaggtgggtgaagaaaacccaataaaataaaagtctgagaagacaaagctagtgaatgaatgtggtatcccggaacccaactgatgtcaagcagaagggtgttatcaactaggtcaaatgctcattcatcaagtaagatgaaactgttataattaaccggtgtcttctgaaatacggagataactcgtgacttaatgaaagcaatagtagagaaggtcaaacttgaccagaatgaaattagaaagaataagaggaaagaaaagaccaaatacagacaaccattgatgccttattcttttgatatactcctggagtccacttgctaatacaattgacccttaaacaatacaggcttgaactgcatgggtccacttatttgtgaattttttttcagttaatacattggaaaatttttggggttttttgacaatttgaaaaaactcacaaactgtctagcctagaaataccgagaaaattaagaaaaagtaagatatgccatgaatgcataaaatatatgtagacactagcctattttatcatttgctactataaaatatacacaatctattataaaaagttaaaatttatcaaaacttaacacacactaacacctaccctacctggcaccattcacagtaaagagaaatgtaaataaacataaaaatgtagtattaaaccataatggcataaaactaattgtagtacatatggtactactgtaataatttggaagccacttcctgttgctattacggtaagctcaagcattgtggatagccatttaaaacaccacgtgatgctaatcatctccgtgtgagcagttctctctccagtaaattgcatattgcagtaaaaagtgatctctagtggttctcgcatatttttcatcatgtttagtgcaatgccataaaccttgaataacatcaagcaatccatacaaagtgccactagtgatgcacggaaaagttgtaacagtacaagaaaaaagttgagttgcttggtatttaccatatattgaggtctgcagctacagttgcctgcaatttcgagataaatgaacccagtataaagactgttgtaacaaaagaaaagaaaatgtgaaaccatcagtgcagctatgccagcaggtgtgaagtcttgcactttttgcaaaatacaaaatatgaaatatgtgttaattgactgtttatgttatctgtaaggtttccactcaacaataggctattagtagttaagtttttgtggagtcaaaaattatacgtggatttttgactatacagtgggttggcacccctaaccttcatgttgataaagggtcaatggtatattatttaatttttttgtatttatattcataaataagattaaatctatatttccaagtaatctctataagattttgttattaatattactattatttttgagacagagtcttactgtcaccaggctggagcacagtggtgcgatctcggctcactgcaacctctgcctcccgggctcaagcaattctcctgcctcaccctcccaagtagctgggactacaggcacgcacaaccacactcagctaatttttgtatttttagtagagacggggtttcaccatgttggccaggatggtattgatctcttgacctcatgatctgcctgcctcggcctcccaaagtgttgggattacaggcatgagccactgtgcacagccattaatattattgttacccaataaaaaaaatttggaaacttgtcttcttttcccctgattctgtttaaatagcactggagttacctgttttgaattttttttccaagcggtcccttatgagttttctctatgttttatttgtttcatttctttttttttttttttttttttttttgagacggagtctcgctctgtcgcccaggctggagtgcagtggcgggatctcggctcactgcaagctccgcctcccgggttcacgccattctcctgcctcagcctcccaagtagctgggactacaggcgcccgccactacgcccggctaattttttgtatttttagtagagacggggtttcaccgttttagccgggatggtctcgatctcctgacctcgtgatccgcccgcctcggcctcccaaagtgctgggattacaggcgtgagccaccgcgcccggcctgtttcatttcttatatcgtatttttgcaactcctttattgatacttttcttcctgattaggtttctactaaaaccaaacaagctttccatgaattagcttttagatttacttattagtttaactgttctgttgtattgtaactcattaatttataattttatctttattaattattctatttttcttcgcttttttgttgtttttctagtttttgagttagatgtttgacgcttttttaaaaagctgtgcattttcctctgggtaatactttagctgtatattatgtattctgatatatagtgtttccattacattgttttctagaaaatctgtagctttgatttatatttgtttcctctttgacctaagatatcctaagggaaaatttaacattttccagaaagaaaacaaattttctttgttttccaagaatgttgttcaaattatttctactgcttggaatttttatcatttttgtgtatccagtaaatagtcaatatttgtacttgctctctgaccacataaaagaatatattcgtgtagtttctattaatagattagagttcaattcagatattaaatgtacatcattattcatgatatttaggtcttctacatcttcacttatcttttttctacttgctttgccattaacagataaagttgaattaaaggcttctactacatacatttctccctgttattccttataggttctgtaatttttgcttcaagaatattgctttttaaatttaatatatagatacttataattacactctagcattataaagagccttttctttttcattgaatgtatttgggcctgcatatgtctaacatgaaaattatagtcctttttttgtttctttgtttgtatttacagttttaagttccattttcaacctttatgcactctttgctttaggtgtgtctcttttagttagcataaagttaggtttgtctttaatttcacctgaagtcttttcctcttaatagatgggttaagccaactgaaaaataaaactgacttatatacttttatttcaagtatgtcctccacaaatattttttgaatagattagcttatatactttggaatttgttaaaaaaagatttttataaaaaataattgtggtgaaatgtacataacataaaatttatcattttgaccatttttaagggcatagctctgtggcataaagtatactcacatagttgtgcaactatcacctccttttgatttttttttactaattttgtaaatttgtttcatctgagctgtcttattatgttttgttttatgtttttctttcctttattatgaagtcactgtattgtctgtaggctatatgtatctgtgagtgtgtgtgtatatgtgtgtattatggtttttaaaaaagtctatatttgttttccagtggctatacttaatactaataactttatgttaaatttttcattctatgtgactctagttcactaatatgagctctgataaaatcagtgctttttcgaggttaggagatcaagaccatcctggctaacacagtgaaactccgtctctactaaaaatacaaaaaattagccagacgtgatggcgggtgcccgtagtcccagctactcgggaggctgaggcaggagaatggcgtgaacccaggaggcagaacttgcagtgagccgagatcgcgccactgcactctagcctgggtgacagagtgagactctgtctctaaataaataaataaataaataaataaataaataaaatcagtgctttttcttcctctgctacctcctttccttctactcagttttagtcagtagtattatcttttttcagatttatctttgtattgttaaatctgcttatgcttctattactttatttattagctttaaatgataccttttgactttcagcttttcttaataaagcaatcagcaaatttcctttacactccacacttataccccatttcctttgtttgtttatttggtttttacttctaacttttcttattgtcaggacatataacatatttaaactttgtttttcaactcgaattctgccattagttttaatttttgttcacagttatataaatctttgttcactgatagtccttttgtactatcatctcttaaatgactttatactccaagaaaggctcatgggaacaatattacctgaatatgtctctattacttaatctgtacctaataatatgaaggtaatctactttgtaggatttctgtgaagattaaataaattaatatagttaaagcacatagaacagcactcgacacagagtgagcacttggcaactgttagctgttactaacctttcccattcttcctccaaacctattccaactatctgaatcatgtgccccttctctgtgaacctctatcataatacttgtcacactgtattgtaattgtctcttttactttcccttgtatcttttgtgcatagcagagtacctgaaacaggaagtattttaaatattttgaatcaaatgagttaatagaatctttacaaataagaatatacacttctgcttaggatgataattggaggcaagtgaatcctgagcgtgatttgataatgacctaataatgatgggttttatttccag

SEQ ID NO: 39 - RNA, 22 nt, Artificial sequence
  Synthetic polynucleotide
ccauucccucuuuagccagagc

SEQ ID NO: 40 - RNA, 22 nt, Artificial sequence
  Synthetic polynucleotide
uggcgagcauucaauaacuuua

SEQ ID NO: 41 - RNA, 22 nt, Artificial sequence
  Synthetic polynucleotide
guucugguuaagaauggauaaa

SEQ ID NO: 42 - RNA, 22 nt, Artificial sequence
  Synthetic polynucleotide
aaaaugucuguucugguuaaga

SEQ ID NO: 43 - RNA, 22 nt, Artificial sequence
  Synthetic polynucleotide
ugaaaaaaugucuguucugguu

SEQ ID NO: 44 - RNA, 22 nt, Artificial sequence
  Synthetic polynucleotide
auaguuuuuugaggaaauacau

SEQ ID NO: 45 - RNA, 22 nt, Artificial sequence
  Synthetic polynucleotide
ccagguuaaguuguucuuaggg

SEQ ID NO: 46 - RNA, 22 nt, Artificial sequence
  Synthetic polynucleotide
auaugccagguuaaguuguucu

SEQ ID NO: 47 - RNA, 22 nt, Artificial sequence
  Synthetic polynucleotide
agaagacuuuguagugaucuuc

SEQ ID NO: 48 - RNA, 22 nt, Artificial sequence
  Synthetic polynucleotide
augagaaagagcuuucuaguau

SEQ ID NO: 49 - RNA, 22 nt, Artificial sequence
  Synthetic polynucleotide
uauugauaaauaauugccuuuc

SEQ ID NO: 50 - RNA, 22 nt, Artificial sequence
  Synthetic polynucleotide
agaugaggagaaccauauuuug

SEQ ID NO: 51 - RNA, 22 nt, Artificial sequence
  Synthetic polynucleotide
acccagccugacaccaaauuua

SEQ ID NO: 52 - RNA, 22 nt, Artificial sequence
  Synthetic polynucleotide
acucaauaaauuuggugucagg

SEQ ID NO: 53 - RNA, 22 nt, Artificial sequence
  Synthetic polynucleotide
auaaguuagcaauggucuaaac

SEQ ID NO: 54 - RNA, 22 nt, Artificial sequence
  Synthetic polynucleotide
aaaagguaguaauuguguuuca

SEQ ID NO: 55 - RNA, 22 nt, Artificial sequence
  Synthetic polynucleotide
gaauugaucuugaagacauacg

SEQ ID NO: 56 - RNA, 22 nt, Artificial sequence
  Synthetic polynucleotide
agcuauuuuugcacaucuauca

SEQ ID NO: 57 - RNA, 22 nt, Artificial sequence
  Synthetic polynucleotide
gauagaugugcaaaaauagcuu

SEQ ID NO: 58 - RNA, 22 nt, Artificial sequence
  Synthetic polynucleotide
aaauuuaauugccaguaagucu

SEQ ID NO: 59 - RNA, 22 nt, Artificial sequence
  Synthetic polynucleotide
uuaugaaaagcagcuaugaagg

SEQ ID NO: 60 - RNA, 22 nt, Artificial sequence
  Synthetic polynucleotide
uagaggaaggcaguggucccuu

SEQ ID NO: 61 - RNA, 22 nt, Artificial sequence
  Synthetic polynucleotide
uacuaaaaggcagccuccuaga

SEQ ID NO: 62 - RNA, 22 nt, Artificial sequence
  Synthetic polynucleotide
gaggcugccuuuuaguaguauu

SEQ ID NO: 63 - RNA, 22 nt, Artificial sequence
  Synthetic polynucleotide
aagaucuagcuaaaauauaaga

SEQ ID NO: 64 - RNA, 22 nt, Artificial sequence
  Synthetic polynucleotide
acaaauuaccaaauuguauuga

SEQ ID NO: 65 - RNA, 22 nt, Artificial sequence
  Synthetic polynucleotide
cauaagaucucugcaaacauau

SEQ ID NO: 66 - RNA, 22 nt, Artificial sequence
  Synthetic polynucleotide
cacugcuuucaggagccaaaaa

SEQ ID NO: 67 - RNA, 22 nt, Artificial sequence
  Synthetic polynucleotide
gggugggggagccccauaaaug

SEQ ID NO: 68 - RNA, 22 nt, Artificial sequence
  Synthetic polynucleotide
agccccauaaauguugaauaau

SEQ ID NO: 69 - RNA, 22 nt, Artificial sequence
  Synthetic polynucleotide
ucagccaauaaaagguuuguua

SEQ ID NO: 70 - RNA, 22 nt, Artificial sequence
  Synthetic polynucleotide
gguuuguuaaagaaugacugug

SEQ ID NO: 71 - RNA, 22 nt, Artificial sequence
  Synthetic polynucleotide
augauauacucuuaagugaaua

SEQ ID NO: 72 - RNA, 22 nt, Artificial sequence
  Synthetic polynucleotide
gccuagaugauuauuaauaggg

SEQ ID NO: 73 - RNA, 22 nt, Artificial sequence
  Synthetic polynucleotide
gccuagaugauuauuaauaggg

SEQ ID NO: 74 - RNA, 22 nt, Artificial sequence
  Synthetic polynucleotide
uuaauaaucaucuaggcugcaa

SEQ ID NO: 75 - RNA, 22 nt, Artificial sequence
  Synthetic polynucleotide
cuaggcacuuguugacaguccu

SEQ ID NO: 76 - RNA, 22 nt, Artificial sequence
  Synthetic polynucleotide
gucuugcgcuuaugaaacuucc

SEQ ID NO: 77 - RNA, 22 nt, Artificial sequence
  Synthetic polynucleotide
uuuuagugaagcaauauuagua

SEQ ID NO: 78 - RNA, 22 nt, Artificial sequence
  Synthetic polynucleotide
auuaguauagaauuuugcauca

SEQ ID NO: 79 - RNA, 22 nt, Artificial sequence
  Synthetic polynucleotide
uauggcaucucaccagugugug

SEQ ID NO: 80 - RNA, 22 nt, Artificial sequence
  Synthetic polynucleotide
auaauagagacaaggugguguc

SEQ ID NO: 81 - RNA, 22 nt, Artificial sequence
  Synthetic polynucleotide
ugaucauugccucacuauggua

SEQ ID NO: 82 - RNA, 22 nt, Artificial sequence
  Synthetic polynucleotide
acauaugagucccaguuauuac

SEQ ID NO: 83 - RNA, 22 nt, Artificial sequence
  Synthetic polynucleotide
uaauaacugggacucauaugua

SEQ ID NO: 84 - RNA, 22 nt, Artificial sequence
  Synthetic polynucleotide
gcuaauagcuuauucuauacau

SEQ ID NO: 85 - RNA, 22 nt, Artificial sequence
  Synthetic polynucleotide
uucuuauuacaugggaaaaaau

SEQ ID NO: 86 - RNA, 22 nt, Artificial sequence
  Synthetic polynucleotide
uucagauuuuuucccauguaau

SEQ ID NO: 87 - RNA, 22 nt, Artificial sequence
  Synthetic polynucleotide
uguuacaugaccuuccuuucuu

SEQ ID NO: 88 - RNA, 22 nt, Artificial sequence
  Synthetic polynucleotide
auuggcuaccuugguuggauga

SEQ ID NO: 89 - RNA, 22 nt, Artificial sequence
  Synthetic polynucleotide
uuaccuggauuggcuaccuugg

SEQ ID NO: 90 - RNA, 22 nt, Artificial sequence
  Synthetic polynucleotide
uuguuacauaaaaagagaggug

SEQ ID NO: 91 - RNA, 22 nt, Artificial sequence
  Synthetic polynucleotide
auaugugcaaguccucugugcu

SEQ ID NO: 92 - RNA, 22 nt, Artificial sequence
  Synthetic polynucleotide
acuugcacauauguucagacug

SEQ ID NO: 93 - RNA, 22 nt, Artificial sequence
  Synthetic polynucleotide
ggugagggucucucuaaggugu

SEQ ID NO: 94 - RNA, 22 nt, Artificial sequence
  Synthetic polynucleotide
uucucacauggcagaaaggggu

SEQ ID NO: 95 - RNA, 22 nt, Artificial sequence
  Synthetic polynucleotide
agcaggaagacagcuggcuauc

SEQ ID NO: 96 - RNA, 22 nt, Artificial sequence
  Synthetic polynucleotide
gacagcuggcuauccaggauuc

SEQ ID NO: 97 - RNA, 22 nt, Artificial sequence
  Synthetic polynucleotide
ucugcuaagagacuccugaauc

SEQ ID NO: 98 - RNA, 22 nt, Artificial sequence
  Synthetic polynucleotide
auuugggucugcuaagagacuc

SEQ ID NO: 99 - RNA, 22 nt, Artificial sequence
  Synthetic polynucleotide
cauagaauauccauccauauga

SEQ ID NO: 100 - RNA, 22 nt, Artificial sequence
  Synthetic polynucleotide
ugcauaguauucuaucauaugg

SEQ ID NO: 101 - RNA, 22 nt, Artificial sequence
  Synthetic polynucleotide
uggauggauauucuaugauaua

SEQ ID NO: 102 - RNA, 22 nt, Artificial sequence
  Synthetic polynucleotide
auuuagaguguauggcaugagu

SEQ ID NO: 103 - RNA, 22 nt, Artificial sequence
  Synthetic polynucleotide
cacucuauuuagaguguauggc

SEQ ID NO: 104 - RNA, 22 nt, Artificial sequence
  Synthetic polynucleotide
uacucaugccauacacucuaaa

SEQ ID NO: 105 - RNA, 22 nt, Artificial sequence
  Synthetic polynucleotide
guggggaaaucagcacucuauu

SEQ ID NO: 106 - RNA, 22 nt, Artificial sequence
  Synthetic polynucleotide
aucauuucaacuuauacacagg

SEQ ID NO: 107 - RNA, 22 nt, Artificial sequence
  Synthetic polynucleotide
uucucucuauauaaagugaucc

SEQ ID NO: 108 - RNA, 22 nt, Artificial sequence
  Synthetic polynucleotide
cauuaggaaaauguacaaagga

SEQ ID NO: 109 - RNA, 22 nt, Artificial sequence
  Synthetic polynucleotide
acuugcaggaggugagggauua

SEQ ID NO: 110 - RNA, 22 nt, Artificial sequence
  Synthetic polynucleotide
auuagggaaugcagacucuggg

SEQ ID NO: 111 - RNA, 22 nt, Artificial sequence
  Synthetic polynucleotide
cugcuacuuggaagacucuucc

SEQ ID NO: 112 - RNA, 22 nt, Artificial sequence
  Synthetic polynucleotide
ggcuggcauagaguaagacagg

SEQ ID NO: 113 - RNA, 22 nt, Artificial sequence
  Synthetic polynucleotide
gaaguaaucggcgguggaggua

SEQ ID NO: 114 - RNA, 22 nt, Artificial sequence
  Synthetic polynucleotide
ugggugagauuagaggccacug

SEQ ID NO: 115 - RNA, 22 nt, Artificial sequence
  Synthetic polynucleotide
ugcuuccucccuugucucccua

SEQ ID NO: 116 - RNA, 22 nt, Artificial sequence
  Synthetic polynucleotide
acuauacuacuuccacuacuac

SEQ ID NO: 117 - RNA, 22 nt, Artificial sequence
  Synthetic polynucleotide
uucaacuuauccauuaauccua

SEQ ID NO: 118 - RNA, 22 nt, Artificial sequence
  Synthetic polynucleotide
gaaggaagauccaauaggauua

SEQ ID NO: 119 - RNA, 22 nt, Artificial sequence
  Synthetic polynucleotide
uggcauaugagaaaagucacag

SEQ ID NO: 120 - RNA, 22 nt, Artificial sequence
  Synthetic polynucleotide
aagauaaugggagaaacagguu

SEQ ID NO: 121 - RNA, 22 nt, Artificial sequence
  Synthetic polynucleotide
uacauccaacuaacaucucuau

SEQ ID NO: 122 - RNA, 22 nt, Artificial sequence
  Synthetic polynucleotide
gacauccaaauagagauguuag

SEQ ID NO: 123 - RNA, 22 nt, Artificial sequence
  Synthetic polynucleotide
ggcugguagugugaagaugggg

SEQ ID NO: 124 - RNA, 22 nt, Artificial sequence
  Synthetic polynucleotide
uucuuaucaacccucuuuccuu

SEQ ID NO: 125 - RNA, 22 nt, Artificial sequence
  Synthetic polynucleotide
aagaggguugauaagaagagaa

SEQ ID NO: 126 - RNA, 22 nt, Artificial sequence
  Synthetic polynucleotide
ugauaagaagagaaaggggugu

SEQ ID NO: 127 - RNA, 22 nt, Artificial sequence
  Synthetic polynucleotide
caccuccuuaaccucuaauacu

SEQ ID NO: 128 - RNA, 22 nt, Artificial sequence
  Synthetic polynucleotide
cuaaguauuagagguuaaggag

SEQ ID NO: 129 - RNA, 22 nt, Artificial sequence
  Synthetic polynucleotide
ucugagaagacaaagcuaguga

SEQ ID NO: 130 - RNA, 22 nt, Artificial sequence
  Synthetic polynucleotide
cugcuugacaucaguuggguuc

SEQ ID NO: 131 - RNA, 22 nt, Artificial sequence
  Synthetic polynucleotide
acacccuucugcuugacaucag

SEQ ID NO: 132 - RNA, 22 nt, Artificial sequence
  Synthetic polynucleotide
gaacccaacugaugucaagcag

SEQ ID NO: 133 - RNA, 22 nt, Artificial sequence
  Synthetic polynucleotide
cuacuauugcuuucauuaaguc

SEQ ID NO: 134 - RNA, 22 nt, Artificial sequence
  Synthetic polynucleotide
aguagagaaggucaaacuugac

SEQ ID NO: 135 - RNA, 22 nt, Artificial sequence
  Synthetic polynucleotide
acuugaccagaaugaaauuaga

SEQ ID NO: 136 - RNA, 22 nt, Artificial sequence
  Synthetic polynucleotide
guggacuccaggaguauaucaa

SEQ ID NO: 137 - RNA, 22 nt, Artificial sequence
  Synthetic polynucleotide
ccuuauucuuuugauauacucc

SEQ ID NO: 138 - RNA, 22 nt, Artificial sequence
  Synthetic polynucleotide
auuguauuagcaaguggacucc

SEQ ID NO: 139 - RNA, 22 nt, Artificial sequence
  Synthetic polynucleotide
guauuucuaggcuagacaguuu

SEQ ID NO: 140 - RNA, 22 nt, Artificial sequence
  Synthetic polynucleotide
cuuuacugugaauggugccagg

SEQ ID NO: 141 - RNA, 22 nt, Artificial sequence
  Synthetic polynucleotide
uauuacgguaagcucaagcauu

SEQ ID NO: 142 - RNA, 22 nt, Artificial sequence
  Synthetic polynucleotide
cuuaacuacuaauagccuauug

SEQ ID NO: 143 - RNA, 22 nt, Artificial sequence
  Synthetic polynucleotide
accccuaaccuucauguugaua

SEQ ID NO: 144 - RNA, 22 nt, Artificial sequence
  Synthetic polynucleotide
aauagcacuggaguuaccuguu

SEQ ID NO: 145 - RNA, 22 nt, Artificial sequence
  Synthetic polynucleotide
uuuuuuuuccaagcggucccuu

SEQ ID NO: 146 - RNA, 22 nt, Artificial sequence
  Synthetic polynucleotide
uggaaaauguuaaauuuucccu

SEQ ID NO: 147 - RNA, 22 nt, Artificial sequence
  Synthetic polynucleotide
uacuugcucucugaccacauaa

SEQ ID NO: 148 - RNA, 22 nt, Artificial sequence
  Synthetic polynucleotide
cuaaucuauuaauagaaacuac

SEQ ID NO: 149 - RNA, 22 nt, Artificial sequence
  Synthetic polynucleotide
agauguagaagaccuaaauauc

SEQ ID NO: 150 - RNA, 22 nt, Artificial sequence
  Synthetic polynucleotide
aagaaaaggcucuuuauaaugc

SEQ ID NO: 151 - RNA, 22 nt, Artificial sequence
  Synthetic polynucleotide
uaaaagagacacaccuaaagca

SEQ ID NO: 152 - RNA, 22 nt, Artificial sequence
  Synthetic polynucleotide
ugaagucuuuuccucuuaauag

SEQ ID NO: 153 - RNA, 22 nt, Artificial sequence
  Synthetic polynucleotide
auauuagugaacuagagucaca

SEQ ID NO: 154 - RNA, 22 nt, Artificial sequence
  Synthetic polynucleotide
aucagagcucauauuagugaac

SEQ ID NO: 155 - RNA, 22 nt, Artificial sequence
  Synthetic polynucleotide
agcacauagaacagcacucgac

SEQ ID NO: 156 - RNA, 22 nt, Artificial sequence
  Synthetic polynucleotide
guugccaagugcucacucugug

SEQ ID NO: 157 - RNA, 22 nt, Artificial sequence
  Synthetic polynucleotide
ccuccaaaccuauuccaacuau

SEQ ID NO: 158 - RNA, 22 nt, Artificial sequence
  Synthetic polynucleotide
aggggcacaugauucagauagu

SEQ ID NO: 159 - RNA, 80 nt, Artificial sequence; identical to SEQ ID NO: 12
  Synthetic polynucleotide

SEQ ID NO: 160 - DNA, 21 bp, Artificial sequence
  Synthetic polynucleotide
gctaccagtgtgatggagtag

SEQ ID NO: 161 - DNA, 25 bp, Artificial sequence
  Synthetic polynucleotide
agccagggatacaatatcttcacaa

SEQ ID NO: 162 - DNA, 20 bp, Artificial sequence
  Synthetic polynucleotide
ccagtcactgactaaccccg

SEQ ID NO: 163 - DNA, 20 bp, Artificial sequence
  Synthetic polynucleotide
actctgtccagagacacgcg

SEQ ID NO: 164 - DNA, 20 bp, Artificial sequence
  Synthetic polynucleotide
ccagtcactgactaaccccg

SEQ ID NO: 165 - DNA, 23 bp, Artificial sequence
  Synthetic polynucleotide
aacttctaatggtgatgacagcc

SEQ ID NO: 166 - DNA, 22 bp, Artificial sequence
  Synthetic polynucleotide
gtcatgattttcctggaccagc

SEQ ID NO: 167 - DNA, 22 bp, Artificial sequence
  Synthetic polynucleotide
gtcatgattttcctggaccagc

SEQ ID NO: 168 - DNA, 22 bp, Artificial sequence
  Synthetic polynucleotide
gtcatgattttcctggaccagc

SEQ ID NO: 169 - DNA, 22 bp, Artificial sequence
  Synthetic polynucleotide
gtcatgattttcctggaccagc

SEQ ID NO: 170 - DNA, 22 bp, Artificial sequence
  Synthetic polynucleotide
gtcatgattttcctggaccagc

SEQ ID NO: 171 - DNA, 22 bp, Artificial sequence
  Synthetic polynucleotide
gtcatgattttcctggaccagc

SEQ ID NO: 172 - DNA, 22 bp, Artificial sequence
  Synthetic polynucleotide
gggtacagtggtgcaatcatgg

SEQ ID NO: 173 - DNA, 22 bp, Artificial sequence
  Synthetic polynucleotide
gggtacagtggtgcaatcatgg

SEQ ID NO: 174 - DNA, 22 bp, Artificial sequence
  Synthetic polynucleotide
gggtacagtggtgcaatcatgg

SEQ ID NO: 175 - DNA, 22 bp, Artificial sequence
  Synthetic polynucleotide
gggtacagtggtgcaatcatgg

SEQ ID NO: 176 - DNA, 22 bp, Artificial sequence
  Synthetic polynucleotide
gggtacagtggtgcaatcatgg

SEQ ID NO: 177 - DNA, 22 bp, Artificial sequence
  Synthetic polynucleotide
gggtacagtggtgcaatcatgg

SEQ ID NO: 178 - DNA, 22 bp, Artificial sequence
  Synthetic polynucleotide
aaaaggcagcctcctagattga

SEQ ID NO: 179 - DNA, 22 bp, Artificial sequence
  Synthetic polynucleotide
aaaaggcagcctcctagattga

SEQ ID NO: 180 - DNA, 22 bp, Artificial sequence
  Synthetic polynucleotide
aaaaggcagcctcctagattga

SEQ ID NO: 181 - DNA, 22 bp, Artificial sequence
  Synthetic polynucleotide
aaaaggcagcctcctagattga

SEQ ID NO: 182 - DNA, 22 bp, Artificial sequence
  Synthetic polynucleotide
aaaaggcagcctcctagattga

SEQ ID NO: 183 - DNA, 22 bp, Artificial sequence
  Synthetic polynucleotide
aaaaggcagcctcctagattga

SEQ ID NO: 184 - DNA, 25 bp, Artificial sequence
  Synthetic polynucleotide
tgcgtatgtcttcaagatcaattct

SEQ ID NO: 185 - DNA, 25 bp, Artificial sequence
  Synthetic polynucleotide
tgcgtatgtcttcaagatcaattct

SEQ ID NO: 186 - DNA, 25 bp, Artificial sequence
  Synthetic polynucleotide
tgcgtatgtcttcaagatcaattct

SEQ ID NO: 187 - DNA, 25 bp, Artificial sequence
  Synthetic polynucleotide
tgcgtatgtcttcaagatcaattct

SEQ ID NO: 188 - DNA, 25 bp, Artificial sequence
  Synthetic polynucleotide
tgcgtatgtcttcaagatcaattct

SEQ ID NO: 189 - DNA, 25 bp, Artificial sequence
  Synthetic polynucleotide
tgcgtatgtcttcaagatcaattct

SEQ ID NO: 190 - DNA, 25 bp, Artificial sequence
  Synthetic polynucleotide
tgcgtatgtcttcaagatcaattct

SEQ ID NO: 191 - DNA, 34 bp, Artificial sequence
  Synthetic polynucleotide
ttggacaaaagatctagctaaaatataagattga

SEQ ID NO: 192 - DNA, 34 bp, Artificial sequence; identical to SEQ ID NO: 191
  Synthetic polynucleotide

SEQ ID NO: 193 - DNA, 34 bp, Artificial sequence; identical to SEQ ID NO: 191
  Synthetic polynucleotide

SEQ ID NO: 194 - DNA, 34 bp, Artificial sequence; identical to SEQ ID NO: 191
  Synthetic polynucleotide

SEQ ID NO: 195 - DNA, 34 bp, Artificial sequence; identical to SEQ ID NO: 191
  Synthetic polynucleotide

SEQ ID NO: 196 - DNA, 34 bp, Artificial sequence; identical to SEQ ID NO: 191
  Synthetic polynucleotide

SEQ ID NO: 197 - DNA, 27 bp, Artificial sequence
  Synthetic polynucleotide
ggcatccagtgatgcaaaattctatac

SEQ ID NO: 198 - DNA, 27 bp, Artificial sequence
  Synthetic polynucleotide
ggcatccagtgatgcaaaattctatac

SEQ ID NO: 199 - DNA, 27 bp, Artificial sequence
  Synthetic polynucleotide
ggcatccagtgatgcaaaattctatac

SEQ ID NO: 200 - DNA, 27 bp, Artificial sequence
  Synthetic polynucleotide
ggcatccagtgatgcaaaattctatac

SEQ ID NO: 201 - DNA, 25 bp, Artificial sequence
  Synthetic polynucleotide
agttgttatctctgaaattttgggt

SEQ ID NO: 202 - DNA, 25 bp, Artificial sequence
  Synthetic polynucleotide
agttgttatctctgaaattttgggt

SEQ ID NO: 203 - DNA, 25 bp, Artificial sequence
  Synthetic polynucleotide
agttgttatctctgaaattttgggt

SEQ ID NO: 204 - DNA, 25 bp, Artificial sequence
  Synthetic polynucleotide
agttgttatctctgaaattttgggt

SEQ ID NO: 205 - DNA, 25 bp, Artificial sequence
  Synthetic polynucleotide
acaatatagctctgataatccaggt

SEQ ID NO: 206 - DNA, 25 bp, Artificial sequence
  Synthetic polynucleotide
acaatatagctctgataatccaggt

SEQ ID NO: 207 - DNA, 25 bp, Artificial sequence
  Synthetic polynucleotide
acaatatagctctgataatccaggt

SEQ ID NO: 208 - DNA, 25 bp, Artificial sequence
  Synthetic polynucleotide
acaatatagctctgataatccaggt

SEQ ID NO: 209 - DNA, 25 bp, Artificial sequence
  Synthetic polynucleotide
acaatatagctctgataatccaggt

SEQ ID NO: 210 - DNA, 18 bp, Artificial sequence
  Synthetic polynucleotide
ccccaccctgcaatacca

SEQ ID NO: 211 - DNA, 18 bp, Artificial sequence
  Synthetic polynucleotide
ccccaccctgcaatacca

SEQ ID NO: 212 - DNA, 18 bp, Artificial sequence
  Synthetic polynucleotide
ccccaccctgcaatacca

SEQ ID NO: 213 - DNA, 18 bp, Artificial sequence
  Synthetic polynucleotide
ccccaccctgcaatacca

SEQ ID NO: 214 - DNA, 18 bp, Artificial sequence
  Synthetic polynucleotide
ccccaccctgcaatacca

SEQ ID NO: 215 - DNA, 18 bp, Artificial sequence
  Synthetic polynucleotide
cctgcattccctcatcca

SEQ ID NO: 216 - DNA, 18 bp, Artificial sequence
  Synthetic polynucleotide
cctgcattccctcatcca

SEQ ID NO: 217 - DNA, 18 bp, Artificial sequence
  Synthetic polynucleotide
cctgcattccctcatcca

SEQ ID NO: 218 - DNA, 18 bp, Artificial sequence
  Synthetic polynucleotide
cctgcattccctcatcca

SEQ ID NO: 219 - DNA, 18 bp, Artificial sequence
  Synthetic polynucleotide
cctgcattccctcatcca

SEQ ID NO: 220 - DNA, 18 bp, Artificial sequence
  Synthetic polynucleotide
cctgcattccctcatcca

SEQ ID NO: 221 - DNA, 18 bp, Artificial sequence
  Synthetic polynucleotide
cctgcattccctcatcca

SEQ ID NO: 222 - DNA, 18 bp, Artificial sequence
  Synthetic polynucleotide
cctgcattccctcatcca

SEQ ID NO: 223 - DNA, 18 bp, Artificial sequence
  Synthetic polynucleotide
cctgcattccctcatcca

SEQ ID NO: 224 - DNA, 18 bp, Artificial sequence
  Synthetic polynucleotide
gctgtggggaaatcagca

SEQ ID NO: 225 - DNA, 18 bp, Artificial sequence
  Synthetic polynucleotide
gctgtggggaaatcagca

SEQ ID NO: 226 - DNA, 18 bp, Artificial sequence
  Synthetic polynucleotide
gctgtggggaaatcagca

SEQ ID NO: 227 - DNA, 28 bp, Artificial sequence
  Synthetic polynucleotide
tgcacacacatttaaatagatgcatagt

SEQ ID NO: 228 - DNA, 28 bp, Artificial sequence
  Synthetic polynucleotide
tgcacacacatttaaatagatgcatagt

SEQ ID NO: 229 - DNA, 28 bp, Artificial sequence
  Synthetic polynucleotide
tgcacacacatttaaatagatgcatagt

SEQ ID NO: 230 - DNA, 28 bp, Artificial sequence
  Synthetic polynucleotide
tgcacacacatttaaatagatgcatagt

SEQ ID NO: 231 - DNA, 28 bp, Artificial sequence
  Synthetic polynucleotide
tgcacacacatttaaatagatgcatagt

SEQ ID NO: 232 - DNA, 28 bp, Artificial sequence
  Synthetic polynucleotide
tgcacacacatttaaatagatgcatagt

SEQ ID NO: 233 - DNA, 19 bp, Artificial sequence
  Synthetic polynucleotide
ttgcctgaactggcacaat

SEQ ID NO: 234 - DNA, 19 bp, Artificial sequence
  Synthetic polynucleotide
ttgcctgaactggcacaat

SEQ ID NO: 235 - DNA, 19 bp, Artificial sequence
  Synthetic polynucleotide
ttgcctgaactggcacaat

SEQ ID NO: 236 - DNA, 19 bp, Artificial sequence
  Synthetic polynucleotide
ttgcctgaactggcacaat

SEQ ID NO: 237 - DNA, 19 bp, Artificial sequence
  Synthetic polynucleotide
ttgcaggaggtgagggatt

SEQ ID NO: 238 - DNA, 19 bp, Artificial sequence
  Synthetic polynucleotide
ttgcaggaggtgagggatt

SEQ ID NO: 239 - DNA, 19 bp, Artificial sequence
  Synthetic polynucleotide
ttgcaggaggtgagggatt

SEQ ID NO: 240 - DNA, 19 bp, Artificial sequence
  Synthetic polynucleotide
ttgcaggaggtgagggatt

SEQ ID NO: 241 - DNA, 19 bp, Artificial sequence
  Synthetic polynucleotide
ttgcaggaggtgagggatt

SEQ ID NO: 242 - DNA, 19 bp, Artificial sequence
  Synthetic polynucleotide
ttgcaggaggtgagggatt

SEQ ID NO: 243 - DNA, 19 bp, Artificial sequence
  Synthetic polynucleotide
ttgcaggaggtgagggatt

SEQ ID NO: 244 - DNA, 19 bp, Artificial sequence
  Synthetic polynucleotide
tcagttgggttccgggata

SEQ ID NO: 245 - DNA, 19 bp, Artificial sequence
  Synthetic polynucleotide
tcagttgggttccgggata

SEQ ID NO: 246 - DNA, 19 bp, Artificial sequence
  Synthetic polynucleotide
tcagttgggttccgggata

SEQ ID NO: 247 - DNA, 19 bp, Artificial sequence
  Synthetic polynucleotide
tcagttgggttccgggata

SEQ ID NO: 248 - DNA, 29 bp, Artificial sequence
  Synthetic polynucleotide
gtcacagaggagtcaaagatgattccaag

SEQ ID NO: 249 - DNA, 29 bp, Artificial sequence
  Synthetic polynucleotide
gtcacagaggagtcaaagatgattccaag

SEQ ID NO: 250 - DNA, 29 bp, Artificial sequence
  Synthetic polynucleotide
gtcacagaggagtcaaagatgattccaag

SEQ ID NO: 251 - DNA, 29 bp, Artificial sequence
  Synthetic polynucleotide
gtcacagaggagtcaaagatgattccaag

SEQ ID NO: 252 - DNA, 29 bp, Artificial sequence
  Synthetic polynucleotide
gtcacagaggagtcaaagatgattccaag

SEQ ID NO: 253 - DNA, 29 bp, Artificial sequence
  Synthetic polynucleotide
gtcacagaggagtcaaagatgattccaag

SEQ ID NO: 254 - DNA, 29 bp, Artificial sequence
  Synthetic polynucleotide
gtcacagaggagtcaaagatgattccaag

SEQ ID NO: 255 - DNA, 29 bp, Artificial sequence
  Synthetic polynucleotide
gtcacagaggagtcaaagatgattccaag

SEQ ID NO: 256 - DNA, 29 bp, Artificial sequence
  Synthetic polynucleotide
gtcacagaggagtcaaagatgattccaag

SEQ ID NO: 257 - DNA, 29 bp, Artificial sequence
  Synthetic polynucleotide
gtcacagaggagtcaaagatgattccaag

SEQ ID NO: 258 - DNA, 25 bp, Artificial sequence
  Synthetic polynucleotide
gggtaggtgttagtgtgtgttaagt

SEQ ID NO: 259 - DNA, 25 bp, Artificial sequence
  Synthetic polynucleotide
gggtaggtgttagtgtgtgttaagt

SEQ ID NO: 260 - DNA, 25 bp, Artificial sequence
  Synthetic polynucleotide
gggtaggtgttagtgtgtgttaagt

SEQ ID NO: 261 - DNA, 25 bp, Artificial sequence
  Synthetic polynucleotide
gggtaggtgttagtgtgtgttaagt

SEQ ID NO: 262 - DNA, 25 bp, Artificial sequence
  Synthetic polynucleotide
gggtaggtgttagtgtgtgttaagt

SEQ ID NO: 263 - DNA, 25 bp, Artificial sequence
  Synthetic polynucleotide
gggtaggtgttagtgtgtgttaagt

SEQ ID NO: 264 - DNA, 25 bp, Artificial sequence
  Synthetic polynucleotide
gggtaggtgttagtgtgtgttaagt

SEQ ID NO: 265 - DNA, 20 bp, Artificial sequence
  Synthetic polynucleotide
tacaggcttgaactgcatgg

SEQ ID NO: 266 - DNA, 20 bp, Artificial sequence
  Synthetic polynucleotide
tacaggcttgaactgcatgg

SEQ ID NO: 267 - DNA, 20 bp, Artificial sequence
  Synthetic polynucleotide
ccactagtgatgcacggaaa

SEQ ID NO: 268 - DNA, 20 bp, Artificial sequence
  Synthetic polynucleotide
ccactagtgatgcacggaaa

SEQ ID NO: 269 - DNA, 20 bp, Artificial sequence
  Synthetic polynucleotide
ctctgcctcccgggctcaag

SEQ ID NO: 270 - DNA, 20 bp, Artificial sequence
  Synthetic polynucleotide
ctctgcctcccgggctcaag

SEQ ID NO: 271 - DNA, 20 bp, Artificial sequence
  Synthetic polynucleotide
agctgtgcattttcctctgg

SEQ ID NO: 272 - DNA, 20 bp, Artificial sequence
  Synthetic polynucleotide
agctgtgcattttcctctgg

SEQ ID NO: 273 - DNA, 20 bp, Artificial sequence
  Synthetic polynucleotide
agctgtgcattttcctctgg

SEQ ID NO: 274 - DNA, 20 bp, Artificial sequence
  Synthetic polynucleotide
agctgtgcattttcctctgg

SEQ ID NO: 275 - DNA, 25 bp, Artificial sequence
  Synthetic polynucleotide
tctgttaatggcaaagcaagtagaa

SEQ ID NO: 276 - DNA, 25 bp, Artificial sequence
  Synthetic polynucleotide
tctgttaatggcaaagcaagtagaa

SEQ ID NO: 277 - DNA, 25 bp, Artificial sequence
  Synthetic polynucleotide
tctgttaatggcaaagcaagtagaa

SEQ ID NO: 278 - DNA, 22 bp, Artificial sequence
  Synthetic polynucleotide
gatgggttaagccaactgaaaa

SEQ ID NO: 279 - DNA, 22 bp, Artificial sequence
  Synthetic polynucleotide
gatgggttaagccaactgaaaa

SEQ ID NO: 280 - DNA, 26 bp, Artificial sequence
  Synthetic polynucleotide
attcacttgcctccaattatcatcct

SEQ ID NO: 281 - DNA, 26 bp, Artificial sequence
  Synthetic polynucleotide
attcacttgcctccaattatcatcct

SEQ ID NO: 282 - DNA, 26 bp, Artificial sequence
  Synthetic polynucleotide
attcacttgcctccaattatcatcct

SEQ ID NO: 283 - DNA, 26 bp, Artificial sequence
  Synthetic polynucleotide
attcacttgcctccaattatcatcct

SEQ ID NO: 284 - DNA, 21 bp, Artificial sequence
  Synthetic polynucleotide
tacagcgtaccttcagctcac

SEQ ID NO: 285 - DNA, 21 bp, Artificial sequence
  Synthetic polynucleotide
accactgccttcctctaactc

SEQ ID NO: 286 - DNA, 30 bp, Artificial sequence
  Synthetic polynucleotide
cattgatgagtttggacaaaccacaactag

SEQ ID NO: 287 - DNA, 22 bp, Artificial sequence
  Synthetic polynucleotide
cacatggcgagcattcaataac

SEQ ID NO: 288 - DNA, 21 bp, Artificial sequence
  Synthetic polynucleotide
aggcatgatccatgagaactg

SEQ ID NO: 289 - DNA, 30 bp, Artificial sequence
  Synthetic polynucleotide
aagaaatccgtcctgagtgtttgatcttcc

SEQ ID NO: 290 - DNA, 21 bp, Artificial sequence
  Synthetic polynucleotide
tacagcgtaccttcagctcac

SEQ ID NO: 291 - DNA, 22 bp, Artificial sequence
  Synthetic polynucleotide
ggaagtttcataagcgcaagac

SEQ ID NO: 292 - DNA, 30 bp, Artificial sequence; identical to SEQ ID NO: 286
  Synthetic polynucleotide

SEQ ID NO: 293 - DNA, 20 bp, Artificial sequence
  Synthetic polynucleotide
agaaagactcctgaaaggca

SEQ ID NO: 294 - DNA, 21 bp, Artificial sequence
  Synthetic polynucleotide
aggcatgatccatgagaactg

SEQ ID NO: 295 - DNA, 30 bp, Artificial sequence; identical to SEQ ID NO: 289
  Synthetic polynucleotide

SEQ ID NO: 296 - DNA, 21 bp, Artificial sequence
  Synthetic polynucleotide
tacagcgtaccttcagctcac

SEQ ID NO: 297 - DNA, 23 bp, Artificial sequence
  Synthetic polynucleotide
gagtgtatggcatgagtacgaat

SEQ ID NO: 298 - DNA, 30 bp, Artificial sequence; identical to SEQ ID NO: 286
  Synthetic polynucleotide

SEQ ID NO: 299 - DNA, 23 bp, Artificial sequence
  Synthetic polynucleotide
aattcctgacaccaccttgtctc

SEQ ID NO: 300 - DNA, 21 bp, Artificial sequence
  Synthetic polynucleotide
aggcatgatccatgagaactg

SEQ ID NO: 301 - DNA, 30 bp, Artificial sequence; identical to SEQ ID NO: 289
  Synthetic polynucleotide

SEQ ID NO: 302 - DNA, 21 bp, Artificial sequence
  Synthetic polynucleotide
tacagcgtaccttcagctcac

SEQ ID NO: 303 - DNA, 22 bp, Artificial sequence
  Synthetic polynucleotide
accctccctgtcttactctatg

SEQ ID NO: 304 - DNA, 30 bp, Artificial sequence; identical to SEQ ID NO: 286
  Synthetic polynucleotide

SEQ ID NO: 305 - DNA, 36 bp, Artificial sequence
  Synthetic polynucleotide
atgcatatatatatttttaacctggattatcagagc

SEQ ID NO: 306 - DNA, 21 bp, Artificial sequence
  Synthetic polynucleotide
aggcatgatccatgagaactg

SEQ ID NO: 307 - DNA, 30 bp, Artificial sequence; identical to SEQ ID NO: 289
  Synthetic polynucleotide

SEQ ID NO: 308 - DNA, 21 bp, Artificial sequence
  Synthetic polynucleotide
tacagcgtaccttcagctcac

SEQ ID NO: 309 - DNA, 20 bp, Artificial sequence
  Synthetic polynucleotide
cttcacccacctccttaacc

SEQ ID NO: 310 - DNA, 30 bp, Artificial sequence; identical to SEQ ID NO: 286
  Synthetic polynucleotide

SEQ ID NO: 311 - DNA, 21 bp, Artificial sequence
  Synthetic polynucleotide
gtgagaacacagcaggaagac

SEQ ID NO: 312 - DNA, 21 bp, Artificial sequence
  Synthetic polynucleotide
aggcatgatccatgagaactg

SEQ ID NO: 313 - DNA, 30 bp, Artificial sequence; identical to SEQ ID NO: 289
  Synthetic polynucleotide

SEQ ID NO: 314 - DNA, 21 bp, Artificial sequence
  Synthetic polynucleotide
tacagcgtaccttcagctcac

SEQ ID NO: 315 - DNA, 23 bp, Artificial sequence
  Synthetic polynucleotide
aggcatcaatggttgtctgtatt

SEQ ID NO: 316 - DNA, 30 bp, Artificial sequence; identical to SEQ ID NO: 286
  Synthetic polynucleotide

SEQ ID NO: 317 - DNA, 20 bp, Artificial sequence
  Synthetic polynucleotide
acttcccagcctccagaact

SEQ ID NO: 318 - DNA, 21 bp, Artificial sequence
  Synthetic polynucleotide
aggcatgatccatgagaactg

SEQ ID NO: 319 - DNA, 30 bp, Artificial sequence; identical to SEQ ID NO: 289
  Synthetic polynucleotide

SEQ ID NO: 320 - DNA, 21 bp, Artificial sequence
  Synthetic polynucleotide
tacagcgtaccttcagctcac

SEQ ID NO: 321 - DNA, 27 bp, Artificial sequence
  Synthetic polynucleotide
gtagggtaggtgttagtgtgtgttaag

SEQ ID NO: 322 - DNA, 30 bp, Artificial sequence; identical to SEQ ID NO: 286
  Synthetic polynucleotide

SEQ ID NO: 323 - DNA, 20 bp, Artificial sequence
  Synthetic polynucleotide
gagtgctgatttccccacag

SEQ ID NO: 324 - DNA, 21 bp, Artificial sequence
  Synthetic polynucleotide
aggcatgatccatgagaactg

SEQ ID NO: 325 - DNA, 30 bp, Artificial sequence; identical to SEQ ID NO: 289
  Synthetic polynucleotide

SEQ ID NO: 326 - DNA, 21 bp, Artificial sequence
  Synthetic polynucleotide
tacagcgtaccttcagctcac

SEQ ID NO: 327 - DNA, 19 bp, Artificial sequence
  Synthetic polynucleotide
agctgcactgatggtttca

SEQ ID NO: 328 - DNA, 30 bp, Artificial sequence; identical to SEQ ID NO: 286
  Synthetic polynucleotide

SEQ ID NO: 329 - DNA, 20 bp, Artificial sequence
  Synthetic polynucleotide
tagggagacaagggaggaag

SEQ ID NO: 330 - DNA, 21 bp, Artificial sequence
  Synthetic polynucleotide
aggcatgatccatgagaactg

SEQ ID NO: 331 - DNA, 30 bp, Artificial sequence; identical to SEQ ID NO: 289
  Synthetic polynucleotide

SEQ ID NO: 332 - DNA, 22 bp, Artificial sequence
  Synthetic polynucleotide
tggaagacagaatggaagaaat

SEQ ID NO: 333 - DNA, 17 bp, Artificial sequence
  Synthetic polynucleotide
gtcaggtccaccactga

SEQ ID NO: 334 - DNA, 25 bp, Artificial sequence
  Synthetic polynucleotide
cccccacccccataggcgagatccc

SEQ ID NO: 335 - DNA, 102 bp, Artificial sequence; identical to SEQ ID NO: 26
  Synthetic polynucleotide

SEQ ID NO: 336 - DNA, 4700 bp, Artificial sequence
  Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtattgaggaaataaatttaaagacatgaaagaatcaaattagagatgagaaagagctttctagtattagaatgggctaaagggcaataggtatttgcttcagaagtctataaaatggttccttgttcccatttgattgtcattttagctgtggtactttgtagaaatgtgagaaaaagtttagtggtctcttgaagcttttcaaaatactttctagaattataccgaataatctaagacaaacagaaaaagaaagagaggaaggaagaaagaaggaaatgaggaagaaaggaagtaggaggaaggaaggaaggaaagaaggaaggaagtaagagggaagcagtgctgctgctgtaggtaaaaatgttaatgaaaatagaaattaagaaagactcctgaaaggcaattatttatcaatatctaagatgaggagaaccatattttgaagaattgaatatgagacttgggaaacaaaatgccacaaaaaatttccactcaataatatacacttctgcttaggatgataattggaggcaagtgaatcctgagcgtgatttgataatgacctaataatgatgggttttatttccagacttctctgctgatggtcatcatgggcgagctggaacccagtgaggggaagatcaaacactcaggacggatttctttttgcagtcagttctcatggatcatgcctgggaccattaaggagaatatcatttttggagtgtcctacgatgaataccggtacagaagcgtgatcaaggcctgccagctggaggaagacattagcaagttcgcagaaaaagataacatcgtgctgggggagggcgggattactctgagtggaggccagcgggccagaatctcactggctcgcgcagtgtacaaggacgctgatctgtatctgctggactctcccttcggctacctggacgtgctgaccgagaaagaaatcttcgagagttgcgtctgtaagctgatggctaacaaaacccggattctggtgacatcaaagatggaacacctgaagaaagcagacaaaatcctgattctgcatgagggctcaagctacttttatgggaccttcagcgaactgcagaatctgcagcccgatttttcctctaagctgatgggatgtgactcctttgatcagttctctgccgaaaggcgcaactccatcctgactgagaccctgcacagattcagcctggaaggcgacgctcccgtgagctggacagagactaagaaacagtcttttaagcagacaggcgagttcggggaaaagcgaaaaaatagcatcctgaacccaatcaatagtattcggaagttctcaatcgtgcagaaaactcccctgcagatgaacggcattgaggaagactccgatgagccactggaacgacggctgagcctggtgcccgattccgagcagggagaagccatcctgcctaggatcagcgtcatttccactggcccaaccctgcaggctagaaggcgccagagtgtgctgaatctgatgacacactcagtcaaccagggccagaatatccatcggaagactaccgcctctacaagaaaagtgagtctggctccacaggcaaacctgactgagctggacatctacagccggcggctgtcccaggagaccgggctggaaatttctgaggaaatcaatgaggaagatctgaaggaatgctttttcgacgatatggagagtatccccgccgtgacaacttggaacacttacctgcgctatattaccgtccacaagtctctgatttttgtcctgatctggtgtctggtcatcttcctggctgaggtcgcagccagcctggtggtcctgtggctgctgggaaacaccccactgcaggacaaggggaattctacacatagtagaaacaatagctacgccgtgatcattacctccacaagttcatactatgtcttctacatctatgtgggcgtcgctgatacactgctggcaatggggtttttcaggggactgcctctggtgcacacactgatcactgtctctaagattctgcaccataaaatgctgcattctgtgctgcaggctccaatgagtaccctgaacacactgaaggcagggggaatcctgaatcggtttagcaaagacatcgccattctggacgatctgctgcctctgaccatttttgatttcatccagctgctgctgatcgtgattggagcaatcgctgtggtcgccgtgctgcagccttacattttcgtcgctactgtgccagtcattgtggccttcatcatgctgcgcgcctatttcctgcagaccagccagcagctgaagcagctggagtctgaaggccggagtccaatctttacacacctggtgacttccctgaaaggactgtggaccctgagagccttcggcaggcagccctactttgagacactgttccacaaggctctgaacctgcatactgcaaattggtttctgtatctgtctaccctgcgatggtttcagatgcggatcgagatgattttcgtgatctttttcattgccgtcaccttcatcagcattctgaccacaggggagggagaaggcagagtgggcatcattctgactctggccatgaacatcatgagtaccctgcagtgggctgtgaatagctccattgacgtggattcactgatgcgctcagtcagccgagtgtttaagttcatcgacatgcccacagaggggaagcctactaaatctaccaagccctacaaaaacggacagctgagcaaagtgatgatcattgaaaattcccatgtcaagaaagacgacatctggcctagcggcgggcagatgaccgtgaaggatctgaccgctaaatacacagaaggaggcaacgcaattctggagaatatctccttttctattagtccaggacagcgagtgggactgctgggacgaacagggtcaggaaagagcactctgctgtccgcattcctgaggctgctgaatactgagggagaaatccagattgacggcgtgtcctgggattctatcaccctgcagcagtggagaaaggcttttggagtcatccctcagaaagtgtttattttcagcggcacattcaggaagaacctggacccatacgaacagtggtccgatcaggagatctggaaagtcgcagacgaagtgggactgcgctctgtgattgaacagtttcctgggaagctggacttcgtcctggtggatgggggatgcgtgctgagccacggccataaacagctgatgtgcctggcccggagtgtgctgtcaaaggctaaaatcctgctgctggacgagccaagcgcccacctggaccccgtgacctaccagatcattagaaggacactgaagcaggcatttgccgactgcaccgtgatcctgtgcgagcatcgcattgaagctatgctggagtgccagcagttcctggtcatcgaggaaaacaaggtccggcagtatgactctattcagaaactgctgaatgagcggagtctgtttagacaggccatctcacccagcgatagggtgaagctgttccctcaccgcaactctagtaagtgtaaatccaagccacagattgccgcactgaaggaagagactgaagaggaggtccaggatacaagactgtgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttatttggtgtcaggctgggtgcagtggctcacacttgtaatcctagcacttttggaggcagaggcaggtgaattgcttgagtccaggagtttgagaccagcgtgggcaacatggcaaaccccacctctacaaaaaacacaaacaaaagaaaatagctgggtgtggtggtgtgtgcctgtagtcccagctacttgggaggctgaggtgggaggatcacctgagcctgagaagtggaggctgcagtgagccatgattgcaccactgtaccctagcctaggtgataggctcaaaaaaaaaaaaaattggtgtttgcaatgctaataatacaatttggttgtttctctctccagttgttttcctacatacgaaacagcttttaaaacaaaatagctggaattgtgcattttttcttacaaaaacattttctttcttaaaatgttattatttttcttttatatcttgtatattattactagcagtgttcactattaaaaaattataggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 337 - DNA, 102 bp, Artificial sequence; identical to SEQ ID NO: 27
  Synthetic polynucleotide

SEQ ID NO: 338 - DNA, 4700 bp, Artificial sequence
  Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtcagcttttaaaacaaaatagctggaattgtgcattttttcttacaaaaacattttctttcttaaaatgttattatttttcttttatatcttgtatattattactagcagtgttcactattaaaaaattatactataggaggggctgatactaaataagttagcaatggtctaaacaaggatgtttatttatgaaaaggtagtaattgtgtttcatagaatttttaaaattaattctgcgtatgtcttcaagatcaattctatgatagatgtgcaaaaatagctttggaattacaaattccaagacttactggcaattaaatttcaggcagttttattaaaattgatgagcagataattactggctgacagtgcagttatagcttatgaaaagcagctatgaaggcagagttagaggaaggcagtggtcccttgggaatatttaaacacttctgagaaacggagtttactaactcaatctaggaggctgccttttagtagttatacacttctgcttaggatgataattggaggcaagtgaatcctgagcgtgatttgataatgacctaataatgatgggttttatttccagacttctctgctgatggtcatcatgggcgagctggaacccagtgaggggaagatcaaacactcaggacggatttctttttgcagtcagttctcatggatcatgcctgggaccattaaggagaatatcatttttggagtgtcctacgatgaataccggtacagaagcgtgatcaaggcctgccagctggaggaagacattagcaagttcgcagaaaaagataacatcgtgctgggggagggcgggattactctgagtggaggccagcgggccagaatctcactggctcgcgcagtgtacaaggacgctgatctgtatctgctggactctcccttcggctacctggacgtgctgaccgagaaagaaatcttcgagagttgcgtctgtaagctgatggctaacaaaacccggattctggtgacatcaaagatggaacacctgaagaaagcagacaaaatcctgattctgcatgagggctcaagctacttttatgggaccttcagcgaactgcagaatctgcagcccgatttttcctctaagctgatgggatgtgactcctttgatcagttctctgccgaaaggcgcaactccatcctgactgagaccctgcacagattcagcctggaaggcgacgctcccgtgagctggacagagactaagaaacagtcttttaagcagacaggcgagttcggggaaaagcgaaaaaatagcatcctgaacccaatcaatagtattcggaagttctcaatcgtgcagaaaactcccctgcagatgaacggcattgaggaagactccgatgagccactggaacgacggctgagcctggtgcccgattccgagcagggagaagccatcctgcctaggatcagcgtcatttccactggcccaaccctgcaggctagaaggcgccagagtgtgctgaatctgatgacacactcagtcaaccagggccagaatatccatcggaagactaccgcctctacaagaaaagtgagtctggctccacaggcaaacctgactgagctggacatctacagccggcggctgtcccaggagaccgggctggaaatttctgaggaaatcaatgaggaagatctgaaggaatgctttttcgacgatatggagagtatccccgccgtgacaacttggaacacttacctgcgctatattaccgtccacaagtctctgatttttgtcctgatctggtgtctggtcatcttcctggctgaggtcgcagccagcctggtggtcctgtggctgctgggaaacaccccactgcaggacaaggggaattctacacatagtagaaacaatagctacgccgtgatcattacctccacaagttcatactatgtcttctacatctatgtgggcgtcgctgatacactgctggcaatggggtttttcaggggactgcctctggtgcacacactgatcactgtctctaagattctgcaccataaaatgctgcattctgtgctgcaggctccaatgagtaccctgaacacactgaaggcagggggaatcctgaatcggtttagcaaagacatcgccattctggacgatctgctgcctctgaccatttttgatttcatccagctgctgctgatcgtgattggagcaatcgctgtggtcgccgtgctgcagccttacattttcgtcgctactgtgccagtcattgtggccttcatcatgctgcgcgcctatttcctgcagaccagccagcagctgaagcagctggagtctgaaggccggagtccaatctttacacacctggtgacttccctgaaaggactgtggaccctgagagccttcggcaggcagccctactttgagacactgttccacaaggctctgaacctgcatactgcaaattggtttctgtatctgtctaccctgcgatggtttcagatgcggatcgagatgattttcgtgatctttttcattgccgtcaccttcatcagcattctgaccacaggggagggagaaggcagagtgggcatcattctgactctggccatgaacatcatgagtaccctgcagtgggctgtgaatagctccattgacgtggattcactgatgcgctcagtcagccgagtgtttaagttcatcgacatgcccacagaggggaagcctactaaatctaccaagccctacaaaaacggacagctgagcaaagtgatgatcattgaaaattcccatgtcaagaaagacgacatctggcctagcggcgggcagatgaccgtgaaggatctgaccgctaaatacacagaaggaggcaacgcaattctggagaatatctccttttctattagtccaggacagcgagtgggactgctgggacgaacagggtcaggaaagagcactctgctgtccgcattcctgaggctgctgaatactgagggagaaatccagattgacggcgtgtcctgggattctatcaccctgcagcagtggagaaaggcttttggagtcatccctcagaaagtgtttattttcagcggcacattcaggaagaacctggacccatacgaacagtggtccgatcaggagatctggaaagtcgcagacgaagtgggactgcgctctgtgattgaacagtttcctgggaagctggacttcgtcctggtggatgggggatgcgtgctgagccacggccataaacagctgatgtgcctggcccggagtgtgctgtcaaaggctaaaatcctgctgctggacgagccaagcgcccacctggaccccgtgacctaccagatcattagaaggacactgaagcaggcatttgccgactgcaccgtgatcctgtgcgagcatcgcattgaagctatgctggagtgccagcagttcctggtcatcgaggaaaacaaggtccggcagtatgactctattcagaaactgctgaatgagcggagtctgtttagacaggccatctcacccagcgatagggtgaagctgttccctcaccgcaactctagtaagtgtaaatccaagccacagattgccgcactgaaggaagagactgaagaggaggtccaggatacaagactgtgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttattaggaatggaacactttatagttttttttggacaaaagatctagctaaaatataagattgaataattgaaaatattaacattttaagttaaatcttacccactcaatacaatttggtaatttgtatcagaagcttaaaagataacctaatagttcttctacttctataacttacccaaatatgtttgcagagatcttatgtaaagctcttcattataacactgctttcaggagccaaaaattgggtgggggagccccataaatgttgaataataggggtttgattagataaattttggtgtagttctataatggcgtgttattcagccaataaaaggtttgttaaagaatgactgtgacggatgtatatgatatactcttaagtgaataaagagttacaaaatgttatgtacaagttacaaaatgtatgtacattatgatccatttttcataaaatcatatgtatgtatatatgtgtgtctggaaggataaatttatcggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 339 - DNA, 102 bp, Artificial sequence; identical to SEQ ID NO: 28
  Synthetic polynucleotide

SEQ ID NO: 340 - DNA, 4700 bp, Artificial sequence
  Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttagagattaggtcttactctgtcacccaggctgaacttcagtggtgtgatcatagctcactgtaaccttgaactcctgggctcaattgacctttccgcttcagcctcccaaagtgctgggtttataggcatgagccactgtgtctggtccaatatgcatatatatatttttaacctggattatcagagctatattgtgtttaggtttataaagctgtactatgtgaaaatatcacttctaggtttaattttgtacaaaggaattttatatagaaatgaggtaattcagattttttcccatgtaataagaattgtaaaatttactgaaacaaacatcaaaaagatatctgttacatgaccttcctttcttttgaatatatttcaggtgatattatttattaaaatttaaaaatgaaaattaaaatatataaaaagttgaaaattattcctttctttactgtctctcatctgtccattttccattctcctgcattccctcatatacacttctgcttaggatgataattggaggcaagtgaatcctgagcgtgatttgataatgacctaataatgatgggttttatttccagacttctctgctgatggtcatcatgggcgagctggaacccagtgaggggaagatcaaacactcaggacggatttctttttgcagtcagttctcatggatcatgcctgggaccattaaggagaatatcatttttggagtgtcctacgatgaataccggtacagaagcgtgatcaaggcctgccagctggaggaagacattagcaagttcgcagaaaaagataacatcgtgctgggggagggcgggattactctgagtggaggccagcgggccagaatctcactggctcgcgcagtgtacaaggacgctgatctgtatctgctggactctcccttcggctacctggacgtgctgaccgagaaagaaatcttcgagagttgcgtctgtaagctgatggctaacaaaacccggattctggtgacatcaaagatggaacacctgaagaaagcagacaaaatcctgattctgcatgagggctcaagctacttttatgggaccttcagcgaactgcagaatctgcagcccgatttttcctctaagctgatgggatgtgactcctttgatcagttctctgccgaaaggcgcaactccatcctgactgagaccctgcacagattcagcctggaaggcgacgctcccgtgagctggacagagactaagaaacagtcttttaagcagacaggcgagttcggggaaaagcgaaaaaatagcatcctgaacccaatcaatagtattcggaagttctcaatcgtgcagaaaactcccctgcagatgaacggcattgaggaagactccgatgagccactggaacgacggctgagcctggtgcccgattccgagcagggagaagccatcctgcctaggatcagcgtcatttccactggcccaaccctgcaggctagaaggcgccagagtgtgctgaatctgatgacacactcagtcaaccagggccagaatatccatcggaagactaccgcctctacaagaaaagtgagtctggctccacaggcaaacctgactgagctggacatctacagccggcggctgtcccaggagaccgggctggaaatttctgaggaaatcaatgaggaagatctgaaggaatgctttttcgacgatatggagagtatccccgccgtgacaacttggaacacttacctgcgctatattaccgtccacaagtctctgatttttgtcctgatctggtgtctggtcatcttcctggctgaggtcgcagccagcctggtggtcctgtggctgctgggaaacaccccactgcaggacaaggggaattctacacatagtagaaacaatagctacgccgtgatcattacctccacaagttcatactatgtcttctacatctatgtgggcgtcgctgatacactgctggcaatggggtttttcaggggactgcctctggtgcacacactgatcactgtctctaagattctgcaccataaaatgctgcattctgtgctgcaggctccaatgagtaccctgaacacactgaaggcagggggaatcctgaatcggtttagcaaagacatcgccattctggacgatctgctgcctctgaccatttttgatttcatccagctgctgctgatcgtgattggagcaatcgctgtggtcgccgtgctgcagccttacattttcgtcgctactgtgccagtcattgtggccttcatcatgctgcgcgcctatttcctgcagaccagccagcagctgaagcagctggagtctgaaggccggagtccaatctttacacacctggtgacttccctgaaaggactgtggaccctgagagccttcggcaggcagccctactttgagacactgttccacaaggctctgaacctgcatactgcaaattggtttctgtatctgtctaccctgcgatggtttcagatgcggatcgagatgattttcgtgatctttttcattgccgtcaccttcatcagcattctgaccacaggggagggagaaggcagagtgggcatcattctgactctggccatgaacatcatgagtaccctgcagtgggctgtgaatagctccattgacgtggattcactgatgcgctcagtcagccgagtgtttaagttcatcgacatgcccacagaggggaagcctactaaatctaccaagccctacaaaaacggacagctgagcaaagtgatgatcattgaaaattcccatgtcaagaaagacgacatctggcctagcggcgggcagatgaccgtgaaggatctgaccgctaaatacacagaaggaggcaacgcaattctggagaatatctccttttctattagtccaggacagcgagtgggactgctgggacgaacagggtcaggaaagagcactctgctgtccgcattcctgaggctgctgaatactgagggagaaatccagattgacggcgtgtcctgggattctatcaccctgcagcagtggagaaaggcttttggagtcatccctcagaaagtgtttattttcagcggcacattcaggaagaacctggacccatacgaacagtggtccgatcaggagatctggaaagtcgcagacgaagtgggactgcgctctgtgattgaacagtttcctgggaagctggacttcgtcctggtggatgggggatgcgtgctgagccacggccataaacagctgatgtgcctggcccggagtgtgctgtcaaaggctaaaatcctgctgctggacgagccaagcgcccacctggaccccgtgacctaccagatcattagaaggacactgaagcaggcatttgccgactgcaccgtgatcctgtgcgagcatcgcattgaagctatgctggagtgccagcagttcctggtcatcgaggaaaacaaggtccggcagtatgactctattcagaaactgctgaatgagcggagtctgtttagacaggccatctcacccagcgatagggtgaagctgttccctcaccgcaactctagtaagtgtaaatccaagccacagattgccgcactgaaggaagagactgaagaggaggtccaggatacaagactgtgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggtttttttccaaccaaggtagccaatccaggtaactttttttagtatcttcccagagatgtttctctctatatatataatcaatatacattttttattattccccacctctctttttatgtaacaatatgcagagttttgcttcttgcttttcccactatcttggacaactttccatattcaaagcacagaggacttgcacatatgttcagactgctgaatatttctgtctctcccctgccattcatatgttgaaatcctaattcccaaggtgatggtattgcagggtggggcctttgggaggtgattagtccatgagggtgaagtctttagtaaatgagattagtgtctttataaaagaaaccttagagagaccctcacaccttagagagaccctcacccctttctgccatgtgagaacacagcaggaagacagctggctatccaggattcaggagtctcttagcagacccaaatctgctggcaccttgatcttggacttcccagcggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 341 - DNA, 102 bp, Artificial sequence; identical to SEQ ID NO: 29
  Synthetic polynucleotide

SEQ ID NO: 342 - DNA, 4700 bp, Artificial sequence
  Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttattcctttctttactgtctctcatctgtccattttccattctcctgcattccctcatccaaccaaggtagccaatccaggtaactttttttagtatcttcccagagatgtttctctctatatatataatcaatatacattttttattattccccacctctctttttatgtaacaatatgcagagttttgcttcttgcttttcccactatcttggacaactttccatattcaaagcacagaggacttgcacatatgttcagactgctgaatatttctgtctctcccctgccattcatatgttgaaatcctaattcccaaggtgatggtattgcagggtggggcctttgggaggtgattagtccatgagggtgaagtctttagtaaatgagattagtgtctttataaaagaaaccttagagagaccctcacaccttagagagaccctcacccctttctgccatgtgagaacacagcaggaagacagctggctatccaggatatacacttctgcttaggatgataattggaggcaagtgaatcctgagcgtgatttgataatgacctaataatgatgggttttatttccagacttctctgctgatggtcatcatgggcgagctggaacccagtgaggggaagatcaaacactcaggacggatttctttttgcagtcagttctcatggatcatgcctgggaccattaaggagaatatcatttttggagtgtcctacgatgaataccggtacagaagcgtgatcaaggcctgccagctggaggaagacattagcaagttcgcagaaaaagataacatcgtgctgggggagggcgggattactctgagtggaggccagcgggccagaatctcactggctcgcgcagtgtacaaggacgctgatctgtatctgctggactctcccttcggctacctggacgtgctgaccgagaaagaaatcttcgagagttgcgtctgtaagctgatggctaacaaaacccggattctggtgacatcaaagatggaacacctgaagaaagcagacaaaatcctgattctgcatgagggctcaagctacttttatgggaccttcagcgaactgcagaatctgcagcccgatttttcctctaagctgatgggatgtgactcctttgatcagttctctgccgaaaggcgcaactccatcctgactgagaccctgcacagattcagcctggaaggcgacgctcccgtgagctggacagagactaagaaacagtcttttaagcagacaggcgagttcggggaaaagcgaaaaaatagcatcctgaacccaatcaatagtattcggaagttctcaatcgtgcagaaaactcccctgcagatgaacggcattgaggaagactccgatgagccactggaacgacggctgagcctggtgcccgattccgagcagggagaagccatcctgcctaggatcagcgtcatttccactggcccaaccctgcaggctagaaggcgccagagtgtgctgaatctgatgacacactcagtcaaccagggccagaatatccatcggaagactaccgcctctacaagaaaagtgagtctggctccacaggcaaacctgactgagctggacatctacagccggcggctgtcccaggagaccgggctggaaatttctgaggaaatcaatgaggaagatctgaaggaatgctttttcgacgatatggagagtatccccgccgtgacaacttggaacacttacctgcgctatattaccgtccacaagtctctgatttttgtcctgatctggtgtctggtcatcttcctggctgaggtcgcagccagcctggtggtcctgtggctgctgggaaacaccccactgcaggacaaggggaattctacacatagtagaaacaatagctacgccgtgatcattacctccacaagttcatactatgtcttctacatctatgtgggcgtcgctgatacactgctggcaatggggtttttcaggggactgcctctggtgcacacactgatcactgtctctaagattctgcaccataaaatgctgcattctgtgctgcaggctccaatgagtaccctgaacacactgaaggcagggggaatcctgaatcggtttagcaaagacatcgccattctggacgatctgctgcctctgaccatttttgatttcatccagctgctgctgatcgtgattggagcaatcgctgtggtcgccgtgctgcagccttacattttcgtcgctactgtgccagtcattgtggccttcatcatgctgcgcgcctatttcctgcagaccagccagcagctgaagcagctggagtctgaaggccggagtccaatctttacacacctggtgacttccctgaaaggactgtggaccctgagagccttcggcaggcagccctactttgagacactgttccacaaggctctgaacctgcatactgcaaattggtttctgtatctgtctaccctgcgatggtttcagatgcggatcgagatgattttcgtgatctttttcattgccgtcaccttcatcagcattctgaccacaggggagggagaaggcagagtgggcatcattctgactctggccatgaacatcatgagtaccctgcagtgggctgtgaatagctccattgacgtggattcactgatgcgctcagtcagccgagtgtttaagttcatcgacatgcccacagaggggaagcctactaaatctaccaagccctacaaaaacggacagctgagcaaagtgatgatcattgaaaattcccatgtcaagaaagacgacatctggcctagcggcgggcagatgaccgtgaaggatctgaccgctaaatacacagaaggaggcaacgcaattctggagaatatctccttttctattagtccaggacagcgagtgggactgctgggacgaacagggtcaggaaagagcactctgctgtccgcattcctgaggctgctgaatactgagggagaaatccagattgacggcgtgtcctgggattctatcaccctgcagcagtggagaaaggcttttggagtcatccctcagaaagtgtttattttcagcggcacattcaggaagaacctggacccatacgaacagtggtccgatcaggagatctggaaagtcgcagacgaagtgggactgcgctctgtgattgaacagtttcctgggaagctggacttcgtcctggtggatgggggatgcgtgctgagccacggccataaacagctgatgtgcctggcccggagtgtgctgtcaaaggctaaaatcctgctgctggacgagccaagcgcccacctggaccccgtgacctaccagatcattagaaggacactgaagcaggcatttgccgactgcaccgtgatcctgtgcgagcatcgcattgaagctatgctggagtgccagcagttcctggtcatcgaggaaaacaaggtccggcagtatgactctattcagaaactgctgaatgagcggagtctgtttagacaggccatctcacccagcgatagggtgaagctgttccctcaccgcaactctagtaagtgtaaatccaagccacagattgccgcactgaaggaagagactgaagaggaggtccaggatacaagactgtgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttttcaggagtctcttagcagacccaaatctgctggcaccttgatcttggacttcccagcctccagaactgtgagaaataaattcctgttgtttataagccacacagttcatggtattttgttatagcagcctgaacaaggacacacacacacacacacacacatgcacacacatttaaatagatgcatagtattctatcatatggatggatattctatgatataatgaatcactattgattgacatttgggttgtttccaatattttgttaacacaaagaacaacactacaaataactttatatacatatcatttagcacatctgcaattgtatcagtaggcttcctataagtggtcaagcatttgtgtacttgtgattttggtagatgttgtcaaatgtccttccctgaaatttgtaccaattcgtactcatgccatacactctaaatagagtgctgatttccccacagcattactaacagatgatattatctaatttaaggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 343 - DNA, 102 bp, Artificial sequence; identical to SEQ ID NO: 30
  Synthetic polynucleotide

SEQ ID NO: 344 - DNA, 4700 bp, Artificial sequence
  Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtacaaagaacaacactacaaataactttatatacatatcatttagcacatctgcaattgtatcagtaggcttcctataagtggtcaagcatttgtgtacttgtgattttggtagatgttgtcaaatgtccttccctgaaatttgtaccaattcgtactcatgccatacactctaaatagagtgctgatttccccacagcattactaacagatgatattatctaatttaaaaagtttctcatcttatagggaaaatagtatgtcaatgtattcttaacttgcatttcttttattataagtagtgtaaaatatcatttcaacttatacacaggaggaatttctctctatataaagtgatcctagaatcataatgaaaaatatcaccaactcattaggaaaatgtacaaaggattgaatagatatctcatcaaaaataaaaatataagtggcctttaaacattgaaaggtaacatttgaacaaagacttgcaggaggtgagggatatacacttctgcttaggatgataattggaggcaagtgaatcctgagcgtgatttgataatgacctaataatgatgggttttatttccagacttctctgctgatggtcatcatgggcgagctggaacccagtgaggggaagatcaaacactcaggacggatttctttttgcagtcagttctcatggatcatgcctgggaccattaaggagaatatcatttttggagtgtcctacgatgaataccggtacagaagcgtgatcaaggcctgccagctggaggaagacattagcaagttcgcagaaaaagataacatcgtgctgggggagggcgggattactctgagtggaggccagcgggccagaatctcactggctcgcgcagtgtacaaggacgctgatctgtatctgctggactctcccttcggctacctggacgtgctgaccgagaaagaaatcttcgagagttgcgtctgtaagctgatggctaacaaaacccggattctggtgacatcaaagatggaacacctgaagaaagcagacaaaatcctgattctgcatgagggctcaagctacttttatgggaccttcagcgaactgcagaatctgcagcccgatttttcctctaagctgatgggatgtgactcctttgatcagttctctgccgaaaggcgcaactccatcctgactgagaccctgcacagattcagcctggaaggcgacgctcccgtgagctggacagagactaagaaacagtcttttaagcagacaggcgagttcggggaaaagcgaaaaaatagcatcctgaacccaatcaatagtattcggaagttctcaatcgtgcagaaaactcccctgcagatgaacggcattgaggaagactccgatgagccactggaacgacggctgagcctggtgcccgattccgagcagggagaagccatcctgcctaggatcagcgtcatttccactggcccaaccctgcaggctagaaggcgccagagtgtgctgaatctgatgacacactcagtcaaccagggccagaatatccatcggaagactaccgcctctacaagaaaagtgagtctggctccacaggcaaacctgactgagctggacatctacagccggcggctgtcccaggagaccgggctggaaatttctgaggaaatcaatgaggaagatctgaaggaatgctttttcgacgatatggagagtatccccgccgtgacaacttggaacacttacctgcgctatattaccgtccacaagtctctgatttttgtcctgatctggtgtctggtcatcttcctggctgaggtcgcagccagcctggtggtcctgtggctgctgggaaacaccccactgcaggacaaggggaattctacacatagtagaaacaatagctacgccgtgatcattacctccacaagttcatactatgtcttctacatctatgtgggcgtcgctgatacactgctggcaatggggtttttcaggggactgcctctggtgcacacactgatcactgtctctaagattctgcaccataaaatgctgcattctgtgctgcaggctccaatgagtaccctgaacacactgaaggcagggggaatcctgaatcggtttagcaaagacatcgccattctggacgatctgctgcctctgaccatttttgatttcatccagctgctgctgatcgtgattggagcaatcgctgtggtcgccgtgctgcagccttacattttcgtcgctactgtgccagtcattgtggccttcatcatgctgcgcgcctatttcctgcagaccagccagcagctgaagcagctggagtctgaaggccggagtccaatctttacacacctggtgacttccctgaaaggactgtggaccctgagagccttcggcaggcagccctactttgagacactgttccacaaggctctgaacctgcatactgcaaattggtttctgtatctgtctaccctgcgatggtttcagatgcggatcgagatgattttcgtgatctttttcattgccgtcaccttcatcagcattctgaccacaggggagggagaaggcagagtgggcatcattctgactctggccatgaacatcatgagtaccctgcagtgggctgtgaatagctccattgacgtggattcactgatgcgctcagtcagccgagtgtttaagttcatcgacatgcccacagaggggaagcctactaaatctaccaagccctacaaaaacggacagctgagcaaagtgatgatcattgaaaattcccatgtcaagaaagacgacatctggcctagcggcgggcagatgaccgtgaaggatctgaccgctaaatacacagaaggaggcaacgcaattctggagaatatctccttttctattagtccaggacagcgagtgggactgctgggacgaacagggtcaggaaagagcactctgctgtccgcattcctgaggctgctgaatactgagggagaaatccagattgacggcgtgtcctgggattctatcaccctgcagcagtggagaaaggcttttggagtcatccctcagaaagtgtttattttcagcggcacattcaggaagaacctggacccatacgaacagtggtccgatcaggagatctggaaagtcgcagacgaagtgggactgcgctctgtgattgaacagtttcctgggaagctggacttcgtcctggtggatgggggatgcgtgctgagccacggccataaacagctgatgtgcctggcccggagtgtgctgtcaaaggctaaaatcctgctgctggacgagccaagcgcccacctggaccccgtgacctaccagatcattagaaggacactgaagcaggcatttgccgactgcaccgtgatcctgtgcgagcatcgcattgaagctatgctggagtgccagcagttcctggtcatcgaggaaaacaaggtccggcagtatgactctattcagaaactgctgaatgagcggagtctgtttagacaggccatctcacccagcgatagggtgaagctgttccctcaccgcaactctagtaagtgtaaatccaagccacagattgccgcactgaaggaagagactgaagaggaggtccaggatacaagactgtgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttttagggaatgcagactctgggaagagtcttccaagtagcaggtgaagcaagtgcaaagctttcagatgggactgactatacctgtctggtttgaagaacagtaaggaggtcactgaggctggcatagagtaagacagggagggtagaatactgtcagagaagtaatcggcggtggaggtagggggtaaaccataaagtgctcgtaaagactaaggcttatttctctgggtgagattagaggccactggagagttttaaacagaagtaacagggccactttggctaatgtttttaggctattctgtagggagacaagggaggaagcaaggagatgagttaggagtctattgtgccagttcaggcaagtgatgatggtggcttgatccaggtagtagtggaagtagtatagtaggaagtgatcagattcaggacatgctttgaaggaagatccaataggattaatggataagttgaacaatggcatatgagaaaagtcacagggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 345 - DNA, 102 bp, Artificial sequence; identical to SEQ ID NO: 31
  Synthetic polynucleotide

SEQ ID NO: 346 - DNA, 4700 bp, Artificial sequence
  Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaataactttatatacatatcatttagcacatctgcaattgtatcagtaggcttcctataagtggtcaagcatttgtgtacttgtgattttggtagatgttgtcaaatgtccttccctgaaatttgtaccaattcgtactcatgccatacactctaaatagagtgctgatttccccacagcattactaacagatgatattatctaatttaaaaagtttctcatcttatagggaaaatagtatgtcaatgtattcttaacttgcatttcttttattataagtagtgtaaaatatcatttcaacttatacacaggaggaatttctctctatataaagtgatcctagaatcataatgaaaaatatcaccaactcattaggaaaatgtacaaaggattgaatagatatctcatcaaaaataaaaatataagtggcctttaaacattgaaaggtaacatttgaacaaagacttgcaggaggtgagggattagggaatgcagactcttatacacttctgcttaggatgataattggaggcaagtgaatcctgagcgtgatttgataatgacctaataatgatgggttttatttccagacttctctgctgatggtcatcatgggcgagctggaacccagtgaggggaagatcaaacactcaggacggatttctttttgcagtcagttctcatggatcatgcctgggaccattaaggagaatatcatttttggagtgtcctacgatgaataccggtacagaagcgtgatcaaggcctgccagctggaggaagacattagcaagttcgcagaaaaagataacatcgtgctgggggagggcgggattactctgagtggaggccagcgggccagaatctcactggctcgcgcagtgtacaaggacgctgatctgtatctgctggactctcccttcggctacctggacgtgctgaccgagaaagaaatcttcgagagttgcgtctgtaagctgatggctaacaaaacccggattctggtgacatcaaagatggaacacctgaagaaagcagacaaaatcctgattctgcatgagggctcaagctacttttatgggaccttcagcgaactgcagaatctgcagcccgatttttcctctaagctgatgggatgtgactcctttgatcagttctctgccgaaaggcgcaactccatcctgactgagaccctgcacagattcagcctggaaggcgacgctcccgtgagctggacagagactaagaaacagtcttttaagcagacaggcgagttcggggaaaagcgaaaaaatagcatcctgaacccaatcaatagtattcggaagttctcaatcgtgcagaaaactcccctgcagatgaacggcattgaggaagactccgatgagccactggaacgacggctgagcctggtgcccgattccgagcagggagaagccatcctgcctaggatcagcgtcatttccactggcccaaccctgcaggctagaaggcgccagagtgtgctgaatctgatgacacactcagtcaaccagggccagaatatccatcggaagactaccgcctctacaagaaaagtgagtctggctccacaggcaaacctgactgagctggacatctacagccggcggctgtcccaggagaccgggctggaaatttctgaggaaatcaatgaggaagatctgaaggaatgctttttcgacgatatggagagtatccccgccgtgacaacttggaacacttacctgcgctatattaccgtccacaagtctctgatttttgtcctgatctggtgtctggtcatcttcctggctgaggtcgcagccagcctggtggtcctgtggctgctgggaaacaccccactgcaggacaaggggaattctacacatagtagaaacaatagctacgccgtgatcattacctccacaagttcatactatgtcttctacatctatgtgggcgtcgctgatacactgctggcaatggggtttttcaggggactgcctctggtgcacacactgatcactgtctctaagattctgcaccataaaatgctgcattctgtgctgcaggctccaatgagtaccctgaacacactgaaggcagggggaatcctgaatcggtttagcaaagacatcgccattctggacgatctgctgcctctgaccatttttgatttcatccagctgctgctgatcgtgattggagcaatcgctgtggtcgccgtgctgcagccttacattttcgtcgctactgtgccagtcattgtggccttcatcatgctgcgcgcctatttcctgcagaccagccagcagctgaagcagctggagtctgaaggccggagtccaatctttacacacctggtgacttccctgaaaggactgtggaccctgagagccttcggcaggcagccctactttgagacactgttccacaaggctctgaacctgcatactgcaaattggtttctgtatctgtctaccctgcgatggtttcagatgcggatcgagatgattttcgtgatctttttcattgccgtcaccttcatcagcattctgaccacaggggagggagaaggcagagtgggcatcattctgactctggccatgaacatcatgagtaccctgcagtgggctgtgaatagctccattgacgtggattcactgatgcgctcagtcagccgagtgtttaagttcatcgacatgcccacagaggggaagcctactaaatctaccaagccctacaaaaacggacagctgagcaaagtgatgatcattgaaaattcccatgtcaagaaagacgacatctggcctagcggcgggcagatgaccgtgaaggatctgaccgctaaatacacagaaggaggcaacgcaattctggagaatatctccttttctattagtccaggacagcgagtgggactgctgggacgaacagggtcaggaaagagcactctgctgtccgcattcctgaggctgctgaatactgagggagaaatccagattgacggcgtgtcctgggattctatcaccctgcagcagtggagaaaggcttttggagtcatccctcagaaagtgtttattttcagcggcacattcaggaagaacctggacccatacgaacagtggtccgatcaggagatctggaaagtcgcagacgaagtgggactgcgctctgtgattgaacagtttcctgggaagctggacttcgtcctggtggatgggggatgcgtgctgagccacggccataaacagctgatgtgcctggcccggagtgtgctgtcaaaggctaaaatcctgctgctggacgagccaagcgcccacctggaccccgtgacctaccagatcattagaaggacactgaagcaggcatttgccgactgcaccgtgatcctgtgcgagcatcgcattgaagctatgctggagtgccagcagttcctggtcatcgaggaaaacaaggtccggcagtatgactctattcagaaactgctgaatgagcggagtctgtttagacaggccatctcacccagcgatagggtgaagctgttccctcaccgcaactctagtaagtgtaaatccaagccacagattgccgcactgaaggaagagactgaagaggaggtccaggatacaagactgtgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttgggaagagtcttccaagtagcaggtgaagcaagtgcaaagctttcagatgggactgactatacctgtctggtttgaagaacagtaaggaggtcactgaggctggcatagagtaagacagggagggtagaatactgtcagagaagtaatcggcggtggaggtagggggtaaaccataaagtgctcgtaaagactaaggcttatttctctgggtgagattagaggccactggagagttttaaacagaagtaacagggccactttggctaatgtttttaggctattctgtagggagacaagggaggaagcaaggagatgagttaggagtctattgtgccagttcaggcaagtgatgatggtggcttgatccaggtagtagtggaagtagtatagtaggaagtgatcagattcaggacatgctttgaaggaagatccaataggattaatggataagttgaacaatggcatatgagaaaagtcacagaggagtcaaagatgattcggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 347 - DNA, 102 bp, Artificial sequence; identical to SEQ ID NO: 32
  Synthetic polynucleotide

SEQ ID NO: 348 - DNA, 4700 bp, Artificial sequence
  Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttagggaaaatagtatgtcaatgtattcttaacttgcatttcttttattataagtagtgtaaaatatcatttcaacttatacacaggaggaatttctctctatataaagtgatcctagaatcataatgaaaaatatcaccaactcattaggaaaatgtacaaaggattgaatagatatctcatcaaaaataaaaatataagtggcctttaaacattgaaaggtaacatttgaacaaagacttgcaggaggtgagggattagggaatgcagactctgggaagagtcttccaagtagcaggtgaagcaagtgcaaagctttcagatgggactgactatacctgtctggtttgaagaacagtaaggaggtcactgaggctggcatagagtaagacagggagggtagaatactgtcagagaagtaatcggcggtggaggtagggggtaaaccataaagtgctcgtaaagactaaggcttatttctctgggtgagattagaggccatatacacttctgcttaggatgataattggaggcaagtgaatcctgagcgtgatttgataatgacctaataatgatgggttttatttccagacttctctgctgatggtcatcatgggcgagctggaacccagtgaggggaagatcaaacactcaggacggatttctttttgcagtcagttctcatggatcatgcctgggaccattaaggagaatatcatttttggagtgtcctacgatgaataccggtacagaagcgtgatcaaggcctgccagctggaggaagacattagcaagttcgcagaaaaagataacatcgtgctgggggagggcgggattactctgagtggaggccagcgggccagaatctcactggctcgcgcagtgtacaaggacgctgatctgtatctgctggactctcccttcggctacctggacgtgctgaccgagaaagaaatcttcgagagttgcgtctgtaagctgatggctaacaaaacccggattctggtgacatcaaagatggaacacctgaagaaagcagacaaaatcctgattctgcatgagggctcaagctacttttatgggaccttcagcgaactgcagaatctgcagcccgatttttcctctaagctgatgggatgtgactcctttgatcagttctctgccgaaaggcgcaactccatcctgactgagaccctgcacagattcagcctggaaggcgacgctcccgtgagctggacagagactaagaaacagtcttttaagcagacaggcgagttcggggaaaagcgaaaaaatagcatcctgaacccaatcaatagtattcggaagttctcaatcgtgcagaaaactcccctgcagatgaacggcattgaggaagactccgatgagccactggaacgacggctgagcctggtgcccgattccgagcagggagaagccatcctgcctaggatcagcgtcatttccactggcccaaccctgcaggctagaaggcgccagagtgtgctgaatctgatgacacactcagtcaaccagggccagaatatccatcggaagactaccgcctctacaagaaaagtgagtctggctccacaggcaaacctgactgagctggacatctacagccggcggctgtcccaggagaccgggctggaaatttctgaggaaatcaatgaggaagatctgaaggaatgctttttcgacgatatggagagtatccccgccgtgacaacttggaacacttacctgcgctatattaccgtccacaagtctctgatttttgtcctgatctggtgtctggtcatcttcctggctgaggtcgcagccagcctggtggtcctgtggctgctgggaaacaccccactgcaggacaaggggaattctacacatagtagaaacaatagctacgccgtgatcattacctccacaagttcatactatgtcttctacatctatgtgggcgtcgctgatacactgctggcaatggggtttttcaggggactgcctctggtgcacacactgatcactgtctctaagattctgcaccataaaatgctgcattctgtgctgcaggctccaatgagtaccctgaacacactgaaggcagggggaatcctgaatcggtttagcaaagacatcgccattctggacgatctgctgcctctgaccatttttgatttcatccagctgctgctgatcgtgattggagcaatcgctgtggtcgccgtgctgcagccttacattttcgtcgctactgtgccagtcattgtggccttcatcatgctgcgcgcctatttcctgcagaccagccagcagctgaagcagctggagtctgaaggccggagtccaatctttacacacctggtgacttccctgaaaggactgtggaccctgagagccttcggcaggcagccctactttgagacactgttccacaaggctctgaacctgcatactgcaaattggtttctgtatctgtctaccctgcgatggtttcagatgcggatcgagatgattttcgtgatctttttcattgccgtcaccttcatcagcattctgaccacaggggagggagaaggcagagtgggcatcattctgactctggccatgaacatcatgagtaccctgcagtgggctgtgaatagctccattgacgtggattcactgatgcgctcagtcagccgagtgtttaagttcatcgacatgcccacagaggggaagcctactaaatctaccaagccctacaaaaacggacagctgagcaaagtgatgatcattgaaaattcccatgtcaagaaagacgacatctggcctagcggcgggcagatgaccgtgaaggatctgaccgctaaatacacagaaggaggcaacgcaattctggagaatatctccttttctattagtccaggacagcgagtgggactgctgggacgaacagggtcaggaaagagcactctgctgtccgcattcctgaggctgctgaatactgagggagaaatccagattgacggcgtgtcctgggattctatcaccctgcagcagtggagaaaggcttttggagtcatccctcagaaagtgtttattttcagcggcacattcaggaagaacctggacccatacgaacagtggtccgatcaggagatctggaaagtcgcagacgaagtgggactgcgctctgtgattgaacagtttcctgggaagctggacttcgtcctggtggatgggggatgcgtgctgagccacggccataaacagctgatgtgcctggcccggagtgtgctgtcaaaggctaaaatcctgctgctggacgagccaagcgcccacctggaccccgtgacctaccagatcattagaaggacactgaagcaggcatttgccgactgcaccgtgatcctgtgcgagcatcgcattgaagctatgctggagtgccagcagttcctggtcatcgaggaaaacaaggtccggcagtatgactctattcagaaactgctgaatgagcggagtctgtttagacaggccatctcacccagcgatagggtgaagctgttccctcaccgcaactctagtaagtgtaaatccaagccacagattgccgcactgaaggaagagactgaagaggaggtccaggatacaagactgtgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttctggagagttttaaacagaagtaacagggccactttggctaatgtttttaggctattctgtagggagacaagggaggaagcaaggagatgagttaggagtctattgtgccagttcaggcaagtgatgatggtggcttgatccaggtagtagtggaagtagtatagtaggaagtgatcagattcaggacatgctttgaaggaagatccaataggattaatggataagttgaacaatggcatatgagaaaagtcacagaggagtcaaagatgattccaagctttctggactgagtaactggaaggataaatgtgccgtttactagaaagataatgggagaaacaggttttggatggagcttggtttgggaatattaagtttgaaatgcctatttgacatccaaatagagatgttagttggatgtacaagtctagtttcaaggaagagggggctggtagtgtgaagatggggctggataagattctaaaggaaagagggttgaggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 349 - DNA, 102 bp, Artificial sequence; identical to SEQ ID NO: 33
  Synthetic polynucleotide

SEQ ID NO: 350 - DNA, 4700 bp, Artificial sequence
  Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttatcatttcaacttatacacaggaggaatttctctctatataaagtgatcctagaatcataatgaaaaatatcaccaactcattaggaaaatgtacaaaggattgaatagatatctcatcaaaaataaaaatataagtggcctttaaacattgaaaggtaacatttgaacaaagacttgcaggaggtgagggattagggaatgcagactctgggaagagtcttccaagtagcaggtgaagcaagtgcaaagctttcagatgggactgactatacctgtctggtttgaagaacagtaaggaggtcactgaggctggcatagagtaagacagggagggtagaatactgtcagagaagtaatcggcggtggaggtagggggtaaaccataaagtgctcgtaaagactaaggcttatttctctgggtgagattagaggccactggagagttttaaacagaagtaacagggccactttggctaatgtttttaggctattctgtagtatacacttctgcttaggatgataattggaggcaagtgaatcctgagcgtgatttgataatgacctaataatgatgggttttatttccagacttctctgctgatggtcatcatgggcgagctggaacccagtgaggggaagatcaaacactcaggacggatttctttttgcagtcagttctcatggatcatgcctgggaccattaaggagaatatcatttttggagtgtcctacgatgaataccggtacagaagcgtgatcaaggcctgccagctggaggaagacattagcaagttcgcagaaaaagataacatcgtgctgggggagggcgggattactctgagtggaggccagcgggccagaatctcactggctcgcgcagtgtacaaggacgctgatctgtatctgctggactctcccttcggctacctggacgtgctgaccgagaaagaaatcttcgagagttgcgtctgtaagctgatggctaacaaaacccggattctggtgacatcaaagatggaacacctgaagaaagcagacaaaatcctgattctgcatgagggctcaagctacttttatgggaccttcagcgaactgcagaatctgcagcccgatttttcctctaagctgatgggatgtgactcctttgatcagttctctgccgaaaggcgcaactccatcctgactgagaccctgcacagattcagcctggaaggcgacgctcccgtgagctggacagagactaagaaacagtcttttaagcagacaggcgagttcggggaaaagcgaaaaaatagcatcctgaacccaatcaatagtattcggaagttctcaatcgtgcagaaaactcccctgcagatgaacggcattgaggaagactccgatgagccactggaacgacggctgagcctggtgcccgattccgagcagggagaagccatcctgcctaggatcagcgtcatttccactggcccaaccctgcaggctagaaggcgccagagtgtgctgaatctgatgacacactcagtcaaccagggccagaatatccatcggaagactaccgcctctacaagaaaagtgagtctggctccacaggcaaacctgactgagctggacatctacagccggcggctgtcccaggagaccgggctggaaatttctgaggaaatcaatgaggaagatctgaaggaatgctttttcgacgatatggagagtatccccgccgtgacaacttggaacacttacctgcgctatattaccgtccacaagtctctgatttttgtcctgatctggtgtctggtcatcttcctggctgaggtcgcagccagcctggtggtcctgtggctgctgggaaacaccccactgcaggacaaggggaattctacacatagtagaaacaatagctacgccgtgatcattacctccacaagttcatactatgtcttctacatctatgtgggcgtcgctgatacactgctggcaatggggtttttcaggggactgcctctggtgcacacactgatcactgtctctaagattctgcaccataaaatgctgcattctgtgctgcaggctccaatgagtaccctgaacacactgaaggcagggggaatcctgaatcggtttagcaaagacatcgccattctggacgatctgctgcctctgaccatttttgatttcatccagctgctgctgatcgtgattggagcaatcgctgtggtcgccgtgctgcagccttacattttcgtcgctactgtgccagtcattgtggccttcatcatgctgcgcgcctatttcctgcagaccagccagcagctgaagcagctggagtctgaaggccggagtccaatctttacacacctggtgacttccctgaaaggactgtggaccctgagagccttcggcaggcagccctactttgagacactgttccacaaggctctgaacctgcatactgcaaattggtttctgtatctgtctaccctgcgatggtttcagatgcggatcgagatgattttcgtgatctttttcattgccgtcaccttcatcagcattctgaccacaggggagggagaaggcagagtgggcatcattctgactctggccatgaacatcatgagtaccctgcagtgggctgtgaatagctccattgacgtggattcactgatgcgctcagtcagccgagtgtttaagttcatcgacatgcccacagaggggaagcctactaaatctaccaagccctacaaaaacggacagctgagcaaagtgatgatcattgaaaattcccatgtcaagaaagacgacatctggcctagcggcgggcagatgaccgtgaaggatctgaccgctaaatacacagaaggaggcaacgcaattctggagaatatctccttttctattagtccaggacagcgagtgggactgctgggacgaacagggtcaggaaagagcactctgctgtccgcattcctgaggctgctgaatactgagggagaaatccagattgacggcgtgtcctgggattctatcaccctgcagcagtggagaaaggcttttggagtcatccctcagaaagtgtttattttcagcggcacattcaggaagaacctggacccatacgaacagtggtccgatcaggagatctggaaagtcgcagacgaagtgggactgcgctctgtgattgaacagtttcctgggaagctggacttcgtcctggtggatgggggatgcgtgctgagccacggccataaacagctgatgtgcctggcccggagtgtgctgtcaaaggctaaaatcctgctgctggacgagccaagcgcccacctggaccccgtgacctaccagatcattagaaggacactgaagcaggcatttgccgactgcaccgtgatcctgtgcgagcatcgcattgaagctatgctggagtgccagcagttcctggtcatcgaggaaaacaaggtccggcagtatgactctattcagaaactgctgaatgagcggagtctgtttagacaggccatctcacccagcgatagggtgaagctgttccctcaccgcaactctagtaagtgtaaatccaagccacagattgccgcactgaaggaagagactgaagaggaggtccaggatacaagactgtgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttggagacaagggaggaagcaaggagatgagttaggagtctattgtgccagttcaggcaagtgatgatggtggcttgatccaggtagtagtggaagtagtatagtaggaagtgatcagattcaggacatgctttgaaggaagatccaataggattaatggataagttgaacaatggcatatgagaaaagtcacagaggagtcaaagatgattccaagctttctggactgagtaactggaaggataaatgtgccgtttactagaaagataatgggagaaacaggttttggatggagcttggtttgggaatattaagtttgaaatgcctatttgacatccaaatagagatgttagttggatgtacaagtctagtttcaaggaagagggggctggtagtgtgaagatggggctggataagattctaaaggaaagagggttgataagaagagaaaggggtgtaggggttagcctaagggcattctaagtattagaggttaaggaggggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 351 - DNA, 102 bp, Artificial sequence; identical to SEQ ID NO: 34
  Synthetic polynucleotide

SEQ ID NO: 352 - DNA, 4700 bp, Artificial sequence
  Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggattagggaatgcagactctgggaagagtcttccaagtagcaggtgaagcaagtgcaaagctttcagatgggactgactatacctgtctggtttgaagaacagtaaggaggtcactgaggctggcatagagtaagacagggagggtagaatactgtcagagaagtaatcggcggtggaggtagggggtaaaccataaagtgctcgtaaagactaaggcttatttctctgggtgagattagaggccactggagagttttaaacagaagtaacagggccactttggctaatgtttttaggctattctgtagggagacaagggaggaagcaaggagatgagttaggagtctattgtgccagttcaggcaagtgatgatggtggcttgatccaggtagtagtggaagtagtatagtaggaagtgatcagattcaggacatgctttgaaggaagatccaataggattaatggataagttgaacaatggcatatgagaaaagtcatatacacttctgcttaggatgataattggaggcaagtgaatcctgagcgtgatttgataatgacctaataatgatgggttttatttccagacttctctgctgatggtcatcatgggcgagctggaacccagtgaggggaagatcaaacactcaggacggatttctttttgcagtcagttctcatggatcatgcctgggaccattaaggagaatatcatttttggagtgtcctacgatgaataccggtacagaagcgtgatcaaggcctgccagctggaggaagacattagcaagttcgcagaaaaagataacatcgtgctgggggagggcgggattactctgagtggaggccagcgggccagaatctcactggctcgcgcagtgtacaaggacgctgatctgtatctgctggactctcccttcggctacctggacgtgctgaccgagaaagaaatcttcgagagttgcgtctgtaagctgatggctaacaaaacccggattctggtgacatcaaagatggaacacctgaagaaagcagacaaaatcctgattctgcatgagggctcaagctacttttatgggaccttcagcgaactgcagaatctgcagcccgatttttcctctaagctgatgggatgtgactcctttgatcagttctctgccgaaaggcgcaactccatcctgactgagaccctgcacagattcagcctggaaggcgacgctcccgtgagctggacagagactaagaaacagtcttttaagcagacaggcgagttcggggaaaagcgaaaaaatagcatcctgaacccaatcaatagtattcggaagttctcaatcgtgcagaaaactcccctgcagatgaacggcattgaggaagactccgatgagccactggaacgacggctgagcctggtgcccgattccgagcagggagaagccatcctgcctaggatcagcgtcatttccactggcccaaccctgcaggctagaaggcgccagagtgtgctgaatctgatgacacactcagtcaaccagggccagaatatccatcggaagactaccgcctctacaagaaaagtgagtctggctccacaggcaaacctgactgagctggacatctacagccggcggctgtcccaggagaccgggctggaaatttctgaggaaatcaatgaggaagatctgaaggaatgctttttcgacgatatggagagtatccccgccgtgacaacttggaacacttacctgcgctatattaccgtccacaagtctctgatttttgtcctgatctggtgtctggtcatcttcctggctgaggtcgcagccagcctggtggtcctgtggctgctgggaaacaccccactgcaggacaaggggaattctacacatagtagaaacaatagctacgccgtgatcattacctccacaagttcatactatgtcttctacatctatgtgggcgtcgctgatacactgctggcaatggggtttttcaggggactgcctctggtgcacacactgatcactgtctctaagattctgcaccataaaatgctgcattctgtgctgcaggctccaatgagtaccctgaacacactgaaggcagggggaatcctgaatcggtttagcaaagacatcgccattctggacgatctgctgcctctgaccatttttgatttcatccagctgctgctgatcgtgattggagcaatcgctgtggtcgccgtgctgcagccttacattttcgtcgctactgtgccagtcattgtggccttcatcatgctgcgcgcctatttcctgcagaccagccagcagctgaagcagctggagtctgaaggccggagtccaatctttacacacctggtgacttccctgaaaggactgtggaccctgagagccttcggcaggcagccctactttgagacactgttccacaaggctctgaacctgcatactgcaaattggtttctgtatctgtctaccctgcgatggtttcagatgcggatcgagatgattttcgtgatctttttcattgccgtcaccttcatcagcattctgaccacaggggagggagaaggcagagtgggcatcattctgactctggccatgaacatcatgagtaccctgcagtgggctgtgaatagctccattgacgtggattcactgatgcgctcagtcagccgagtgtttaagttcatcgacatgcccacagaggggaagcctactaaatctaccaagccctacaaaaacggacagctgagcaaagtgatgatcattgaaaattcccatgtcaagaaagacgacatctggcctagcggcgggcagatgaccgtgaaggatctgaccgctaaatacacagaaggaggcaacgcaattctggagaatatctccttttctattagtccaggacagcgagtgggactgctgggacgaacagggtcaggaaagagcactctgctgtccgcattcctgaggctgctgaatactgagggagaaatccagattgacggcgtgtcctgggattctatcaccctgcagcagtggagaaaggcttttggagtcatccctcagaaagtgtttattttcagcggcacattcaggaagaacctggacccatacgaacagtggtccgatcaggagatctggaaagtcgcagacgaagtgggactgcgctctgtgattgaacagtttcctgggaagctggacttcgtcctggtggatgggggatgcgtgctgagccacggccataaacagctgatgtgcctggcccggagtgtgctgtcaaaggctaaaatcctgctgctggacgagccaagcgcccacctggaccccgtgacctaccagatcattagaaggacactgaagcaggcatttgccgactgcaccgtgatcctgtgcgagcatcgcattgaagctatgctggagtgccagcagttcctggtcatcgaggaaaacaaggtccggcagtatgactctattcagaaactgctgaatgagcggagtctgtttagacaggccatctcacccagcgatagggtgaagctgttccctcaccgcaactctagtaagtgtaaatccaagccacagattgccgcactgaaggaagagactgaagaggaggtccaggatacaagactgtgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcagaggagtcaaagatgattccaagctttctggactgagtaactggaaggataaatgtgccgtttactagaaagataatgggagaaacaggttttggatggagcttggtttgggaatattaagtttgaaatgcctatttgacatccaaatagagatgttagttggatgtacaagtctagtttcaaggaagagggggctggtagtgtgaagatggggctggataagattctaaaggaaagagggttgataagaagagaaaggggtgtaggggttagcctaagggcattctaagtattagaggttaaggaggtgggtgaagaaaacccaataaaataaaagtctgagaagacaaagctagtgaatgaatgtggtatcccggaacccaactgatgtcaagcagaagggtgttatcaactaggtcaaatgctcattcatcaagtaagatgaaactgttataattaaccggtgtcttctgaaatacggagataactcgtgacttaggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 353 - DNA, 102 bp, Artificial sequence; identical to SEQ ID NO: 35
  Synthetic polynucleotide

SEQ ID NO: 354 - DNA, 4700 bp, Artificial sequence
  Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggaatattaagtttgaaatgcctatttgacatccaaatagagatgttagttggatgtacaagtctagtttcaaggaagagggggctggtagtgtgaagatggggctggataagattctaaaggaaagagggttgataagaagagaaaggggtgtaggggttagcctaagggcattctaagtattagaggttaaggaggtgggtgaagaaaacccaataaaataaaagtctgagaagacaaagctagtgaatgaatgtggtatcccggaacccaactgatgtcaagcagaagggtgttatcaactaggtcaaatgctcattcatcaagtaagatgaaactgttataattaaccggtgtcttctgaaatacggagataactcgtgacttaatgaaagcaatagtagagaaggtcaaacttgaccagaatgaaattagaaagaataagaggaaagaaaagaccaaatacagacaaccattgatgccttattcttttgatatactatacacttctgcttaggatgataattggaggcaagtgaatcctgagcgtgatttgataatgacctaataatgatgggttttatttccagacttctctgctgatggtcatcatgggcgagctggaacccagtgaggggaagatcaaacactcaggacggatttctttttgcagtcagttctcatggatcatgcctgggaccattaaggagaatatcatttttggagtgtcctacgatgaataccggtacagaagcgtgatcaaggcctgccagctggaggaagacattagcaagttcgcagaaaaagataacatcgtgctgggggagggcgggattactctgagtggaggccagcgggccagaatctcactggctcgcgcagtgtacaaggacgctgatctgtatctgctggactctcccttcggctacctggacgtgctgaccgagaaagaaatcttcgagagttgcgtctgtaagctgatggctaacaaaacccggattctggtgacatcaaagatggaacacctgaagaaagcagacaaaatcctgattctgcatgagggctcaagctacttttatgggaccttcagcgaactgcagaatctgcagcccgatttttcctctaagctgatgggatgtgactcctttgatcagttctctgccgaaaggcgcaactccatcctgactgagaccctgcacagattcagcctggaaggcgacgctcccgtgagctggacagagactaagaaacagtcttttaagcagacaggcgagttcggggaaaagcgaaaaaatagcatcctgaacccaatcaatagtattcggaagttctcaatcgtgcagaaaactcccctgcagatgaacggcattgaggaagactccgatgagccactggaacgacggctgagcctggtgcccgattccgagcagggagaagccatcctgcctaggatcagcgtcatttccactggcccaaccctgcaggctagaaggcgccagagtgtgctgaatctgatgacacactcagtcaaccagggccagaatatccatcggaagactaccgcctctacaagaaaagtgagtctggctccacaggcaaacctgactgagctggacatctacagccggcggctgtcccaggagaccgggctggaaatttctgaggaaatcaatgaggaagatctgaaggaatgctttttcgacgatatggagagtatccccgccgtgacaacttggaacacttacctgcgctatattaccgtccacaagtctctgatttttgtcctgatctggtgtctggtcatcttcctggctgaggtcgcagccagcctggtggtcctgtggctgctgggaaacaccccactgcaggacaaggggaattctacacatagtagaaacaatagctacgccgtgatcattacctccacaagttcatactatgtcttctacatctatgtgggcgtcgctgatacactgctggcaatggggtttttcaggggactgcctctggtgcacacactgatcactgtctctaagattctgcaccataaaatgctgcattctgtgctgcaggctccaatgagtaccctgaacacactgaaggcagggggaatcctgaatcggtttagcaaagacatcgccattctggacgatctgctgcctctgaccatttttgatttcatccagctgctgctgatcgtgattggagcaatcgctgtggtcgccgtgctgcagccttacattttcgtcgctactgtgccagtcattgtggccttcatcatgctgcgcgcctatttcctgcagaccagccagcagctgaagcagctggagtctgaaggccggagtccaatctttacacacctggtgacttccctgaaaggactgtggaccctgagagccttcggcaggcagccctactttgagacactgttccacaaggctctgaacctgcatactgcaaattggtttctgtatctgtctaccctgcgatggtttcagatgcggatcgagatgattttcgtgatctttttcattgccgtcaccttcatcagcattctgaccacaggggagggagaaggcagagtgggcatcattctgactctggccatgaacatcatgagtaccctgcagtgggctgtgaatagctccattgacgtggattcactgatgcgctcagtcagccgagtgtttaagttcatcgacatgcccacagaggggaagcctactaaatctaccaagccctacaaaaacggacagctgagcaaagtgatgatcattgaaaattcccatgtcaagaaagacgacatctggcctagcggcgggcagatgaccgtgaaggatctgaccgctaaatacacagaaggaggcaacgcaattctggagaatatctccttttctattagtccaggacagcgagtgggactgctgggacgaacagggtcaggaaagagcactctgctgtccgcattcctgaggctgctgaatactgagggagaaatccagattgacggcgtgtcctgggattctatcaccctgcagcagtggagaaaggcttttggagtcatccctcagaaagtgtttattttcagcggcacattcaggaagaacctggacccatacgaacagtggtccgatcaggagatctggaaagtcgcagacgaagtgggactgcgctctgtgattgaacagtttcctgggaagctggacttcgtcctggtggatgggggatgcgtgctgagccacggccataaacagctgatgtgcctggcccggagtgtgctgtcaaaggctaaaatcctgctgctggacgagccaagcgcccacctggaccccgtgacctaccagatcattagaaggacactgaagcaggcatttgccgactgcaccgtgatcctgtgcgagcatcgcattgaagctatgctggagtgccagcagttcctggtcatcgaggaaaacaaggtccggcagtatgactctattcagaaactgctgaatgagcggagtctgtttagacaggccatctcacccagcgatagggtgaagctgttccctcaccgcaactctagtaagtgtaaatccaagccacagattgccgcactgaaggaagagactgaagaggaggtccaggatacaagactgtgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggtttttttcctggagtccacttgctaatacaattgacccttaaacaatacaggcttgaactgcatgggtccacttatttgtgaattttttttcagttaatacattggaaaatttttggggttttttgacaatttgaaaaaactcacaaactgtctagcctagaaataccgagaaaattaagaaaaagtaagatatgccatgaatgcataaaatatatgtagacactagcctattttatcatttgctactataaaatatacacaatctattataaaaagttaaaatttatcaaaacttaacacacactaacacctaccctacctggcaccattcacagtaaagagaaatgtaaataaacataaaaatgtagtattaaaccataatggcataaaactaattgtagtacatatggtactactgtaataatttggaagccacttcctgttgctattacggtaagctcaagcattgtggatagccatttaaaacaccacgtgatgctaatcaggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

(19) United States
(12) Patent Application Publication
(10) Pub. No.: US 2021/0403906 A1
(43) Pub. Date: Dec. 30, 2021

(54) GENE-EDITING SYSTEMS FOR EDITING A CYSTIC FIBROSIS TRANSMEMBRANE REGULATOR (CFTR) GENE

(71) Applicant: Vertex Pharmaceuticals Incorporated, Boston, MA (US)

(72) Inventors: Marco Weinberg, Encinitas, CA (US); Diego D'Astolfo, Boston, MA (US)

(73) Assignee: Vertex Pharmaceuticals Incorporated, Boston, MA (US)

(21) Appl. No.: 17/339,425

(22) Filed: Jun. 4, 2021

Related U.S. Application Data
(63) Continuation of application No. PCT/US2019/064718, filed on Dec. 5, 2019.
(60) Provisional application No. 62/775,637, filed on Dec. 5, 2018.

(51) Int. Cl. C12N 15/11; C12N 9/22; C12N 15/86; C12N 5/071

(52) U.S. Cl. CPC C12N 15/111; C12N 5/0688; C12N 15/86; C12N 9/22

(57) ABSTRACT

Described herein are highly efficient gene-editing systems comprising a nuclease, a guide RNA, and/or a donor template and uses thereof for editing a cystic fibrosis transmembrane regulator (CFTR) gene either in vitro or in vivo.

### RELATED APPLICATION

This application is a continuation of International Patent Application Serial No. PCT/US2019/064718, filed Dec. 5, 2019, which claims the benefit of priority under 35 U.S.C. § 119(e) of the filing date of U.S. Provisional Application Ser. No. 62/775,637, entitled “GENE-EDITING SYSTEMS FOR EDITING A CYSTIC FIBROSIS TRANSMEMBRANE REGULATOR (CFTR) GENE”, filed on Dec. 5, 2018; the entire contents of each of which are incorporated herein by reference.

### BACKGROUND

Cystic fibrosis (CF) is the most common autosomal recessive disease in the Caucasian population. It causes severe damage to the lungs, pancreas, liver, intestines, sinuses, and other organs of the body. Various known mutations within the CF transmembrane conductance regulator (CFTR) gene cause CF. While technological advances have increased the life expectancy of CF patients, there is still no effective cure for the disease. It is therefore of great interest to develop new therapies for CF.

### SUMMARY

The present disclosure is based, at least in part, on the development of efficient gene editing systems for correcting mutations in a CF transmembrane conductance regulator (CFTR) gene. In some embodiments, the gene editing system relies on the identification of effective editing positions in intron 10 of the CFTR gene (e.g., those disclosed herein) for effective insertion of a nucleic acid encoding exons 11 to 27 of the CFTR gene, which would result in the correction of 99% of the most frequent CFTR non-responsive alleles.

As such, in some aspects, the disclosure relates to gene-editing systems for modifying a cystic fibrosis transmembrane regulator (CFTR) gene. Such a gene-editing system may comprise: (a) a first polynucleotide, which comprises a first nucleotide sequence encoding exons 11 to 27 of the CFTR gene; (b) a second polynucleotide, which comprises a second nucleotide sequence encoding an RNA-guided DNA endonuclease, or the RNA-guided DNA endonuclease; and (c) a third polynucleotide, which comprises a third nucleotide sequence encoding a guide RNA (gRNA), wherein the gRNA directs cleavage by the RNA-guided DNA endonuclease at a target site, which is position 1220, 2068, 3821, 4262, 5041, 5052, 5278, 5343, 5538, or 6150 of intron 10 in the CFTR gene. In some embodiments, the first nucleotide of the first polynucleotide of (a) is free of intron sequences.

In some embodiments, the first polynucleotide of (a) may further comprise a 5′ homologous arm upstream to the first nucleotide sequence and/or a 3′ homologous arm downstream to the first nucleotide sequence. The 5′ homologous arm may comprise a nucleic acid sequence that is homologous to a region upstream to the target site. Alternatively or in addition, the 3′ homologous arm may comprise a nucleic acid sequence that is homologous to a region downstream to the target site. In some embodiments, the 5′ homologous arm and the 3′ homologous arm comprise nucleotide sequences selected from the group consisting of:

(i) SEQ ID NO: 17 and SEQ ID NO: 18, respectively;

(ii) SEQ ID NO: 19 and SEQ ID NO: 20, respectively;

(iii) SEQ ID NO: 21 and SEQ ID NO: 22, respectively;

(iv) SEQ ID NO: 23 and SEQ ID NO: 24, respectively;

(v) SEQ ID NO: 25 and SEQ ID NO: 345, respectively;

(vi) SEQ ID NO: 346 and SEQ ID NO: 347, respectively;

(vii) SEQ ID NO: 348 and SEQ ID NO: 349, respectively;

(viii) SEQ ID NO: 350 and SEQ ID NO: 351, respectively;

(ix) SEQ ID NO: 352 and SEQ ID NO: 353, respectively; and

(x) SEQ ID NO: 354 and SEQ ID NO: 355, respectively.

In some embodiments, the first nucleotide sequence in (a) may further comprise a first fragment upstream to the first nucleotide sequence and downstream to the 5′ homologous arm, and wherein the first fragment contains an acceptor splice site. For example, the first fragment may comprise the nucleotide sequence of TATACACTTCTGCTTAGGATGATAATTGGAGGCAAGTGAATCCTGAGCGTGATTTGA TAATGACCTAATAATGATGGGTTTTATTTCCAG (SEQ ID NO: 1), wherein the 3′ end AG is the splicing acceptor site.

In some embodiments, the second nucleotide sequence encoding the RNA-guided DNA endonuclease in (b) may further comprise a nucleotide sequence encoding a nuclear localization signal (NLS), which is fused in-frame with the RNA-guided DNA endonuclease. In some embodiments, the NLS is a SV40 NLS. In some embodiments, the RNA-guided DNA endonuclease can be a Cas9 endonuclease, for example, a Staphylococcus aureus Cas9 enzyme (saCas9).

In some embodiments, the third nucleotide sequence in (c), which encodes the gRNA, may comprise one of the following nucleotide sequences:

| | (i) |
|---|---|
| (SEQ ID NO: 2) | |
| | ACCCAGCCTGACACCAAATTTA; |
| | (ii) |
| (SEQ ID NO: 3) | |
| | TACTAAAAGGCAGCCTCCTAGA; |
| | (iii) |
| (SEQ ID NO: 4) | |
| | ATTGGCTACCTTGGTTGGATGA; |
| | (iv) |
| (SEQ ID NO: 5) | |
| | GACAGCTGGCTATCCAGGATTC; |
| | (v) |
| (SEQ ID NO: 6) | |
| | ACTTGCAGGAGGTGAGGGATTA; |
| | (vi) |
| (SEQ ID NO: 7) | |
| | ATTAGGGAATGCAGACTCTGGG; |
| | (vii) |
| (SEQ ID NO: 8) | |
| | TGGGTGAGATTAGAGGCCACTG; |
| | (viii) |
| (SEQ ID NO: 9) | |
| | TGCTTCCTCCCTTGTCTCCCTA; |
| | (iv) |
| (SEQ ID NO: 10) | |
| | TGGCATATGAGAAAAGTCACAG; |
| | and |
| | (x) |
| (SEQ ID NO: 11) | |
| | CCTTATTCTTTTGATATACTCC. |

It should be understood that because the third nucleotide sequence encoding the gRNA can be either DNA sequences or RNA sequences, any of the thymines (T) in the sequences may be replaced with a uracil (U). In some embodiments, the third nucleotide sequence in (c) may further comprise a scaffold sequence, which in some examples may comprise the nucleotide sequence of

(SEQ ID NO: 12)
GUUUUAGUACUCUGGAAACAGAAUCUACUAAAACAAGGCAAAAUGCCGUG
UUUAUCUCGUCAACUUGUUGGCGAGAUUUU.

In some embodiments, (a), (b), and (c) may be located on the same vector. In other embodiments, (a), (b), and/or (c) may be located on different vectors. For example, (a) and (c) may be located on a first vector, and (b) may be located on a second vector which is different from the first vector. Alternatively, (a) may be located on a first vector, and (b) and (c) may be located on a second vector which is different from the first vector. In some embodiments, the vector(s) is a viral vector(s), for example an adeno-associated viral (AAV) vector(s).

Also within the scope of the present disclosure are viral particles or sets of viral particles, which collectively comprise any of the gene-editing systems disclosed herein. In some embodiments, the viral particle is, or set of viral particles are, AAV particle(s).

In yet other aspects, the disclosure relates to methods of editing a CFTR gene, the method comprises contacting a cell with (i) any of the gene-editing systems disclosed herein or (ii) a viral particle or a set of viral particles, which collectively comprise the gene-editing system. In some embodiments, the cell may comprise a mutation in one or more of exons 11-27 of the CFTR gene. Example mutations include, but are not limited to, F508del, I507del, G542X, S549N, G551D, R553X, D1152H, N1303K, W1282X, 2789+5G>A, or 3849+10kbC>T.

In some embodiments, the contacting step is performed by administering the gene-editing system or the viral particle(s) to a subject in need thereof. In some examples, the gene-editing system or the viral particle(s) is administered to the respiratory tract of the subject. In some embodiments, the subject is a human patient having cystic fibrosis. In some examples, the human patient is a child.

In some embodiments, the cell is a stem cell, for example an iPSC cell or a bronchioalveolar stem cell.

In some examples, any of the methods described herein may further comprise administering the cell with the edited CFTR gene to a subject in need thereof. In some examples, the cell with the edited CFTR gene is administered to the respiratory tract of the subject (e.g., a human patient having cystic fibrosis). In some examples, the human patient may be a child.

In other aspects, the present disclosure relates to nucleic acids, which may be viral vectors such as AAV vectors. The nucleic acid may comprise: (a) a first nucleotide sequence encoding exons 11 to 27 of a CFTR gene; (b) a 5′ homologous arm upstream to the first nucleotide sequence, wherein the 5′ homologous arm comprises a nucleic acid sequence that is homologous to a region upstream to a target position in intron 10 of the CFTR gene; and (c) a 3′ homologous arm downstream to the first nucleotide sequence, wherein the 3′ homologous arm comprises a nucleic acid sequence that is homologous to a region downstream to a target position in intron 10 of the CFTR gene; wherein the target position is selected from the group consisting of position 1220, 2068, 3821, 4262, 5041, 5052, 5278, 5343, 5538, or 6150 of intron 10 in the CFTR gene. In some embodiments, the first nucleotide sequence of (a) is free of intron sequences.

In other embodiments, the nucleic acid may comprise: (a) a first nucleotide sequence encoding an RNA-guided DNA endonuclease, and (b) a second nucleotide sequence encoding a guide RNA (gRNA), wherein the gRNA directs cleavage by the RNA-guided DNA endonuclease at a target position of a CFTR gene, wherein the target position is selected from the group consisting of position 1220, 2068, 3821, 4262, 5041, 5052, 5278, 5343, 5538, or 6150 of intron 10 in the CFTR gene; wherein each of the first nucleotide sequence and the second nucleotide sequence is in operable linkage to a promoter.

In some embodiments, the first nucleotide sequence in (a) may further comprise a first fragment linked to the 5′ end of the nucleotide sequence encoding exons 11 to exon 27 of the CFTR gene, wherein the first fragment comprises a acceptor splice site (e.g., those disclosed herein).

In some embodiments, the nucleic acid may further comprise a second nucleotide sequence encoding a guide RNA (gRNA), wherein the gRNA directs cleavage by the RNA-guided DNA endonuclease at any of the target positions disclosed herein. Exemplary gRNAs are also provided in the present disclosure. See page 14 below.

In other aspects, the present disclosure relates to genetically edited lung cells or precursor cells, thereof. The genetically edited lung cell or the precursor cell, thereof may comprise a genetically edited endogenous CFTR gene, in which an exogenous nucleic acid is inserted into intron 10 of the endogenous CFTR gene, wherein the exogenous nucleic acid comprises a first nucleotide sequence encoding exons 11 to 27 of a CFTR gene. In some embodiments, the first nucleotide sequence is free of intron sequences. In some embodiments, the exogenous nucleic acid further comprises a second nucleotide sequence linked to the 5′ end of the first nucleotide sequence, the second nucleotide sequence comprising an acceptor slice site. In some embodiments, the second nucleotide sequence comprises SEQ ID NO: 1. In some embodiments, the edited lung cell or precursor cell thereof is a human cell. In some embodiments, the precursor cell is an iPSC cell or a bronchioalveolar stem cell.

Also within the scope of the present disclosure are uses of any of the gene-editing systems described herein, components thereof, or any of the genetically engineered cells described herein for treating CF, as well as uses thereof for manufacturing a medicament for the intended medical treatment.

The details of one or more embodiments of the disclosure are set forth in the description below. Other features or advantages of the present disclosure will be apparent from the detailed description of several embodiments and also from the appended claims.

### BRIEF DESCRIPTION OF THE DRAWINGS

FIGS. 1A-1B is a schematic depicting CFTR gene-editing strategies. FIG. 1A depicts a gene-editing strategy based on a dual AAV-based delivery of CRISPR-Cas9 and CFTR super-exon 11-27. The first (1) AAV vector expresses saCAS9 and single molecule gRNA (sgRNA) to induce a specific double-stranded DNA cut at intron 10 of CFTR gene, and the second (2) AAV vector serves as an HDR donor template. FIG. 1B depicts a gene-editing strategy based on a single AAV-based delivery of the HDR donor template and the sgRNA. LHA: left homology arm; RHA: right homology arm.

FIG. 2 is a diagram showing candidate cut sites identified from a 120 saCas9 gRNA Indel screen in electroporated lung progenitor cells (LPCs). The positive and negative controls were cells electroporated with saCAS9 mRNA together VEGFA gRNA or without gRNA, respectively (black and white diamonds). Indel rates (left y-axis) are represented by solid dots, and cell survival rates (right y-axis) are represented by hollow squares. Nucleotide positions are represented on the x-axis, with the first nucleotide of hsCFTR intron 10 designated a value of 1 and so forth. SEQ ID NOs are provided in TABLE 1.

FIG. 3 is a chart showing validation of 10 candidate sgRNA target sites in intron 10 of the CFTR gene (labeled according to FIG. 2).

FIGS. 4A-4B provide charts showing indel patterns of the 10 candidate sgRNA target sites (labeled according to FIG. 2). Heat map graphs show indel pattern profiles from −25 until +25 nucleotides (y-axis) of the CRISPR-CAS9 cut site of each of the 10 candidate sgRNA target sites. White and black values were set as 0% and 100%, respectively.

FIG. 5 is a diagram showing an overview of off-target analysis of the 10 candidate sgRNA target sites. Summary off-target analysis of 10 candidate gRNAs by using GUIDE-Sequencing and Hybrid capture technologies. Asterisk highlights gRNAs with potential and/or significant off-target editing.

FIG. 6 is a schematic depicting an exemplary experimental workflow used to determine functional correction in LPCs following CRISPR-CAS9 mediated CFTR super-exon AAV insertion. LPC: lung progenitor cell; HBE: human bronchial epithelial cell; HDR: homology-dependent recombination.

FIG. 7 is a diagram showing measurements of CFTR super-exon HDR rates for the 10 candidate sgRNA target sites in LPCs. Graph showing rates of homology-dependent recombination (HDR) of CFTR super-exon 11-27 and cell survival. LPCs were electroporated with or without saCAS9 and the corresponding sgRNA (CRISPR/Cas9). Cells were seeded in media containing the corresponding CFTR super-exon AAV donor at the concentrations shown. Cell survival rates are shown in percentages where mock electroporated cells were set arbitrarily at 100%. Graph shows the mean and SD per experimental condition (n=4).

FIG. 8 is a chart showing functional CFTR correction following CRISPR-CAS9 mediated insertion of CFTR super-exon 11-27 donor at 7 sites in HBEs. Graph showing CFTR functional correction and HDR rates of 7 CFTR selected gRNA sites in HBEs. dF508/dF508 LPCs were electroporated with or without saCAS9 mRNA and the corresponding sgRNA (CRISPR/Cas9). Cells were seeded in media with the corresponding CFTR super-exon AAV donor. Positive controls for CFTR function were cells treated with triple combination of small molecule CFTR correctors and potentiators (659-TC). Graph shows the mean and SD per experimental condition (n=3).

### DETAILED DESCRIPTION

Gene editing (including genomic editing) is a type of genetic engineering in which nucleotide(s)/nucleic acid(s) is/are inserted, deleted, and/or substituted in a DNA sequence, such as in the genome of a targeted cell. Targeted gene editing enables insertion, deletion, and/or substitution at pre-selected sites in the genome of a targeted cell (e.g., in a targeted gene or targeted DNA sequence). When a sequence of an endogenous gene is edited, for example by deletion, insertion or substitution of nucleotide(s)/nucleic acid(s), the endogenous gene comprising the affected sequence may be knocked-out or knocked-down due to the sequence alteration. Therefore, targeted editing may be used to disrupt endogenous gene expression. Alternatively or in addition, a desired nucleic acid may be inserted into a target site in a DNA sequence (e.g., in an endogenous gene), which is known as targeted integration. “Targeted integration” refers to a process involving insertion of one or more exogenous sequences, with or without deletion of an endogenous sequence at the insertion site. Targeted integration can result from targeted gene editing when a donor template containing an exogenous sequence is present.

The present disclosure is based, at least in part, on the development of efficient gene editing systems for correcting a mutation(s) in a CF transmembrane conductance regulator (CFTR) gene, e.g., mutations in regions encoded by one of exons 11-27 and cause CF. As described herein, cell survival, indel rates, and indel patterns were determined at 120 previously uncharacterized target positions in intron 10 of the CFTR gene and a number of effective editing positions were identified based on the results. Accordingly, the gene-editing systems described herein rely on the identification of candidate target positions (e.g., those disclosed herein) that facilitate effective insertion (as determined by cell survival, indel rates, and indel patterns) of a nucleic acid encoding exons 11 to 27 of the CFTR gene, which would result in the correction of 99% of the most frequent CFTR non-responsive alleles.

Accordingly, provided herein are gene-editing systems for efficient modification of CFTR genes and uses thereof for correcting mutations in the CFTR gene, thereby treating CF. Components of the gene-editing systems and genetically modified cells resulting from application of the gene-editing systems are also within the scope of the present disclosure.

### I. Gene-Editing Systems for Genetic Modification of a CFTR Gene

In some aspects, the disclosure relates to gene-editing systems for modifying a cystic fibrosis transmembrane regulator (CFTR) gene. A “gene-editing system” refers to a combination of components for genetic editing a target gene (e.g., CFTR), or one or more agents for producing such components. For example, a gene-editing system may comprise: (a) a nuclease, or an agent (e.g., a nucleic acid encoding the nuclease) for producing such; (b) a guide RNA (gRNA), or an agent for producing such (e.g., a vector capable of expressing the gRNA); and/or (c) a donor template, or an agent for producing such (e.g., a vector capable of producing the donor template).

The gene-editing systems as described herein may exhibit one or more advantageous in modifying a CFTR gene. For example, it would achieve a high gene editing rates, such as homology-directed repair rates (e.g., at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 30%, at least 35%, or at least 40% as assessed by methods known in the art (e.g., by methods as described herein)). Further, cells edited by the gene-editing system disclosed herein would have a high survival rate (e.g., at least 50%, at least 55%, at least 60%, at least 65%, at least 70%, at least 75%, at least 80%, at least 85%, at least 85%, at least 90%, at least 95%, or at least 99%) relative to an unedited control. Alternative or in addition, cells edited by the gene-editing system would exhibit an increased CFTR activity (e.g., by at least 30%, 50%, 100%, 2-fold, 5-fold, or 10-fold) relative to the unedited control.

In one exemplary embodiment, a gene-editing system as described herein may comprise: (a) a first polynucleotide, which comprises a nucleotide sequence encoding the donor template; (b) a second polynucleotide, which comprises a nucleotide sequence encoding the RNA-guided DNA endonuclease; and (c) a third polynucleotide, which comprises a nucleotide sequence encoding the gRNA. In another exemplary embodiment, a gene-editing system may comprise: (a) a first polynucleotide, which comprises a nucleotide sequence encoding the donor template; (b) a polypeptide, which comprises the RNA-guided DNA endonuclease; and (c) a third polynucleotide, which comprises a nucleotide sequence encoding the gRNA.

A. Nuclease

Targeted gene editing can be achieved either through a nuclease-independent approach, or through a nuclease-dependent approach. In the nuclease-independent targeted editing approach, homologous recombination is guided by homologous sequences flanking an exogenous polynucleotide to be introduced into an endogenous sequence through the enzymatic machinery of the host cell. The exogenous polynucleotide may introduce deletions, insertions or replacement of nucleotides in the endogenous sequence.

Alternatively, the nuclease-dependent approach can achieve targeted editing through the introduction of double strand breaks (DSBs) at specific locations using sequence-specific nucleases (e.g., endonucleases). Such nuclease-dependent targeted editing also utilizes DNA repair mechanisms, for example, non-homologous end joining (NHEJ), which occurs in response to DSBs. DNA repair by NHEJ often leads to random insertions or deletions (indels) of a small number of endogenous nucleotides. In contrast to NHEJ mediated repair, repair can also occur by a homology directed repair (HDR). When a donor template containing exogenous genetic material flanked by a pair of homology arms is present, the exogenous genetic material can be introduced into the genome by HDR, which results in targeted integration of the exogenous genetic material.

A nuclease of a gene-editing system may be provided in the form of a polypeptide (i.e., an enzymatic form). Alternatively, a gene-editing system may comprise an agent for the production of a nuclease. For example, a gene-editing system may comprise a nucleotide sequence encoding the sequence of a nuclease and an additional nucleotide sequence that facilitates expression/production of the nuclease as a polypeptide.

Available nucleases capable of introducing specific and targeted DSBs include, but not limited to, zinc-finger nucleases (ZFN), transcription activator-like effector nucleases (TALEN), and RNA-guided endonucleases (e.g., CRISPR-Cas9 or CRISPR/Cas9; Clustered Regular Interspaced Short Palindromic Repeats Associated 9 nucleases). Additional examples of targeted nucleases suitable for use as provided herein include, but are not limited to, Bxbl, phiC31, R4, PhiBT1, and Wβ/SPBc/TP901-1, whether used individually or in combination. Other non-limiting examples of targeted nucleases include naturally-occurring and recombinant nucleases, e.g., CRISPR/Cas9, restriction endonucleases, meganucleases homing endonucleases, and the like.

(i) Zinc Finger Nucleases (ZFNs)

ZFNs are targeted nucleases comprising a nuclease fused to a zinc finger DNA binding domain (ZFBD), which is a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but not limited to, C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A selected zinc finger domain is a domain not found in nature whose production results primarily from an empirical process such as phage display, interaction trap or hybrid selection. ZFNs are described in greater detail in U.S. Pat. Nos. 7,888,121 and 7,972,854. The most recognized example of a ZFN is a fusion of the FokI nuclease with a zinc finger DNA binding domain.

(ii) TALEN Nucleases

A TALEN is a targeted nuclease comprising a nuclease fused to a TAL effector DNA binding domain. A “transcription activator-like effector DNA binding domain”, “TAL effector DNA binding domain”, or “TALE DNA binding domain” is a polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in US Patent Application No. 2011/0145940. The most recognized example of a TALEN in the art is a fusion polypeptide of the FokI nuclease to a TAL effector DNA binding domain.

(iii) RNA-Guided Endonucleases

RNA-guided endonucleases are enzymes that utilize RNA:DNA base-pairing to target and cleave a polynucleotide. RNA-guided endonuclease may cleave single-stranded polynucleic acids or at least one strand of a double-stranded polynucleotide. A gene editing-system may comprise one RNA-guided endonuclease. Alternatively, a gene-editing system may comprise at least two (e.g., two, three, four, five, six, seven, eight, nine, ten, or more than ten) RNA-guided endonucleases.

The CRISPR-Cas9 system is a naturally-occurring defense mechanism in prokaryotes that has been repurposed as a RNA-guided DNA-targeting platform used for gene editing. It relies on the DNA nuclease Cas9, and two noncoding RNAs—crisprRNA (crRNA) and trans-activating RNA (tracrRNA)—to target the cleavage of DNA. crRNA drives sequence recognition and specificity of the CRISPR-Cas9 complex through Watson-Crick base pairing typically with a 20 nucleotide (nt) sequence in the target DNA. Changing the sequence of the 5′ 20 nt in the crRNA allows targeting of the CRISPR-Cas9 complex to specific loci. The CRISPR-Cas9 complex only binds DNA sequences that contain a sequence match to the first 20 nt of the crRNA if the target sequence is followed by a specific short DNA motif (with the sequence NGG) referred to as a protospacer adjacent motif (PAM). TracrRNA hybridizes with the 3′ end of crRNA to form an RNA-duplex structure that is bound by the Cas9 endonuclease to form the catalytically active CRISPR-Cas9 complex, which can then cleave the target DNA.

Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end).

A gene-editing system may comprise a CRISPR endonuclease (e.g., a CRISPR associated protein 9 or Cas9 nuclease). In some embodiments, the endonuclease is from Streptococcus aureus (e.g., saCas9), although other CRISPR homologs may be used. It should be understood that a Cas9 may be substituted with another RNA-guided endonuclease known in the art, such as Cpf1. Finally, it should be understood, that a wild-type RNA-guided endonuclease may be used or modified versions may be used (e.g., evolved versions of Cas9, Cas9 orthologues, Cas9 chimeric/fusion proteins, or other Cas9 functional variants). For example, in some embodiments, the RNA-guided endonuclease is modified to comprise a nuclear localization signal (NLS), such as an SV40 NLS or a NucleoPlasmine NLS. Examples of other nuclear localization signals are known to those having skill in the art. In some embodiments, the NLS comprises an SV40 NLS and a NucleoPlasmine NLS.

B. Guide RNA

The present disclosure provides a genome-targeting nucleic acid, or an agent for producing such (e.g., a polynucleotide comprising a nucleotide sequence encoding a gRNA), that can direct the activities of an associated polypeptide (e.g., an RNA-guided endonuclease) to a specific target sequence within a target nucleic acid. The genome-targeting nucleic acid can be an RNA. A genome-targeting RNA is referred to as a “guide RNA” or “gRNA” herein. In some embodiments, a gene-editing systems comprises one gRNA. In other embodiments, a gene-editing system comprises at least two gRNAs (e.g., two, three, four, five, six, seven, eight, nine, ten, or more than ten gRNAs).

A gRNA of a gene-editing system may be provided in a synthesized form. For example, a guide RNA may be synthesized by chemical means, as illustrated below and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

Alternatively, a gene-editing system may comprise an agent for the production of a gRNA. For example, a gene-editing system may comprise a nucleotide sequence encoding the nucleotide sequence of a gRNA and an additional nucleotide sequence that facilitates expression/production of the gRNA.

A gRNA may be a double-molecule guide RNA. A double-molecule gRNA comprises two strands of RNA. The first strand may comprise in the 5′ to 3′ direction, an optional spacer extension sequence, a spacer sequence and a scaffold sequence a minimum CRISPR repeat sequence. The second strand comprises a minimum tracrRNA sequence (complementary to the minimum CRISPR repeat sequence), a 3′ tracrRNA sequence, and an optional tracrRNA extension sequence.

Alternatively, a gRNA may be a single-molecule guide RNA comprising a spacer sequence and a scaffold sequence. A single-molecule guide RNA may further comprise an optional spacer extension.

(i) gRNA Spacer

As is understood by the person of ordinary skill in the art, each gRNA is designed to include a spacer sequence complementary to its genomic target sequence. See Jinek et al., Science, 337, 816-821 (2012) and Deltcheva et al., Nature, 471, 602-607 (2011). A spacer sequence is a sequence (e.g., a 20 nucleotide sequence) that defines the target sequence (e.g., a DNA target sequences, such as a genomic target sequence) of a target nucleic acid of interest. The gRNA can comprise a variable length spacer sequence with 17-30 nucleotides at the 5′ end of the gRNA sequence. In some embodiments, the spacer sequence is 15 to 30 nucleotides. In some embodiments, the spacer sequence is 15, 16, 17, 18, 19, 29, 21, 22, 23, 24, 25, 26, 27, 28, 29, or 30 nucleotides. In some embodiments, a spacer sequence is 20 nucleotides.

The “target sequence” is adjacent to a PAM sequence and is the sequence modified by an RNA-guided nuclease (e.g., Cas9). The “target nucleic acid” is a double-stranded molecule: one strand comprises the target sequence and is referred to as the “PAM strand,” and the other complementary strand is referred to as the “non-PAM strand.” One of skill in the art recognizes that the gRNA spacer sequence hybridizes to the reverse complement of the target sequence, which is located in the non-PAM strand of the target nucleic acid of interest. Thus, the gRNA spacer sequence is the RNA equivalent of the target sequence. For example, if the target sequence is 5′-AGAGCAACAGTGCTGTGGCC-3′ (SEQ ID NO: 13), then the gRNA spacer sequence is 5′-AGAGCAACAGUGCUGUGGCC-3′ (SEQ ID NO: 14). The spacer of a gRNA interacts with a target nucleic acid of interest in a sequence-specific manner via hybridization (i.e., base pairing). The nucleotide sequence of the spacer thus varies depending on the target sequence of the target nucleic acid of interest.

The spacer sequence is designed to hybridize to a region of the target nucleic acid that is located 5′ of a PAM of the Cas9 enzyme used in the system. The spacer may perfectly match the target sequence or may have mismatches. Each Cas9 enzyme has a particular PAM sequence that it recognizes in a target DNA. For example, S. pyogenes Cas9 recognizes in a target nucleic acid a PAM that comprises the sequence 5′-NRG-3′, where R comprises either A or G, where N is any nucleotide and N is immediately 3′ of the target nucleic acid sequence targeted by the spacer sequence.

In some embodiments, the target nucleic acid sequence comprises 20 nucleotides. In some embodiments, the target nucleic acid comprises less than 20 nucleotides. In some embodiments, the target nucleic acid comprises more than 20 nucleotides. In some embodiments, the target nucleic acid comprises at least: 5, 10, 15, 16, 17, 18, 19, 20, 21, 22, 23, 24, 25, 30 or more nucleotides. In some embodiments, the target nucleic acid comprises at most: 5, 10, 15, 16, 17, 18, 19, 20, 21, 22, 23, 24, 25, 30 or more nucleotides. In some embodiments, the target nucleic acid sequence comprises 20 bases immediately 5′ of the first nucleotide of the PAM. For example, in a sequence comprising 5′-NNNNNNNNNNNNNNNNNNNNNRG-3′, the target nucleic acid comprises the sequence that corresponds to the Ns, wherein N is any nucleotide, and the underlined NRG sequence is the S. aureus PAM.

In some embodiments, a gRNA for use in the gene-editing system disclosed herein may direct cleavage by the RNA-guided endonuclease to a target site at position 1220, 2068, 3821, 4262, 5041, 5052, 5278, 5343, 5538, or 6150 of intron 10 in the CFTR gene, wherein the designated number corresponds to the nucleotide positon within intron 10 of the hsCFTR gene (i.e., the first nucleotide of the intron is given a value of 1, the second a value of 2, and so forth). Exemplary gRNAs may comprise one of the following spacer sequences:

| | (i) |
|---|---|
| (SEQ ID NO: 2) | |
| | ACCCAGCCTGACACCAAATTTA; |
| | (ii) |
| (SEQ ID NO: 3) | |
| | TACTAAAAGGCAGCCTCCTAGA; |
| | (iii) |
| (SEQ ID NO: 4) | |
| | ATTGGCTACCTTGGTTGGATGA; |
| | (iv) |
| (SEQ ID NO: 5) | |
| | GACAGCTGGCTATCCAGGATTC; |
| | (v) |
| (SEQ ID NO: 6) | |
| | ACTTGCAGGAGGTGAGGGATTA; |
| | (vi) |
| (SEQ ID NO: 7) | |
| | ATTAGGGAATGCAGACTCTGGG; |
| | (vii) |
| (SEQ ID NO: 8) | |
| | TGGGTGAGATTAGAGGCCACTG; |
| | (viii) |
| (SEQ ID NO: 9) | |
| | TGCTTCCTCCCTTGTCTCCCTA; |
| | (iv) |
| (SEQ ID NO: 10) | |
| | TGGCATATGAGAAAAGTCACAG; |
| | and |
| | (x) |
| (SEQ ID NO: 11) | |
| | CCTTATTCTTTTGATATACTCC. |

(ii) gRNA Scaffold

In some embodiments, the gRNA further comprises a scaffold sequence. A scaffold sequence may comprise the sequence of a minimum CRISPR repeat sequence, a single-molecule guide linker, a minimum tracrRNA sequence, a 3′ tracrRNA sequence, and/or an optional tracrRNA extension sequence. In some embodiments, the scaffold sequence comprises the nucleotide sequence of GUUUUAGUACUCUGGAAACAGAAUCUACUAAAACAAGGCA AAAUGCCGUGUUUAUCUCGUCAACUUGUUGGCGAGAUUUU (SEQ ID NO: 12). The scaffold sequence may be connected to the 5′ and/or 3′ end of a spacer sequence. In some embodiments the scaffold sequence is connected to the 3′ end of the spacer sequence. In other embodiments, the scaffold sequence is connected to the 5′ end of the spacer sequence.

(iii) Exemplary gRNA Sequences

A gRNA for use in the gene-editing system disclosed herein may comprise, consist essentially of (e.g., contain up to 20 extra nucleotides at the 5′ end and/or the 3′ end of the following sequences), or consist of one of the following nucleotide sequences:

(i)
(SEQ ID NO: 26)
acccagcctgacaccaaatttaGUUUUAGUACUCUGGAAACAGAAUCUAC
UAAAACAAGGCAAAAUGCCGUGUUUAUCUCGUCAACUUGUUGGCGAGAUU
UU;
(ii)
(SEQ ID NO: 27)
tactaaaaggcagcctcctagaGUUUUAGUACUCUGGAAACAGAAUCUAC
UAAAACAAGGCAAAAUGCCGUGUUUAUCUCGUCAACUUGUUGGCGAGAUU
UU;
(iii)
(SEQ ID NO: 28)
attggctaccttggttggatgaGUUUUAGUACUCUGGAAACAGAAUCUAC
UAAAACAAGGCAAAAUGCCGUGUUUAUCUCGUCAACUUGUUGGCGAGAUU
UU;
(iv)
(SEQ ID NO: 29)
gacagctggctatccaggattcGUUUUAGUACUCUGGAAACAGAAUCUAC
UAAAACAAGGCAAAAUGCCGUGUUUAUCUCGUCAACUUGUUGGCGAGAUU
UU;
(v)
(SEQ ID NO: 30)
acttgcaggaggtgagggattaGUUUUAGUACUCUGGAAACAGAAUCUAC
UAAAACAAGGCAAAAUGCCGUGUUUAUCUCGUCAACUUGUUGGCGAGAUU
UU;
(vi)
(SEQ ID NO: 31)
attagggaatgcagactctgggGUUUUAGUACUCUGGAAACAGAAUCUAC
UAAAACAAGGCAAAAUGCCGUGUUUAUCUCGUCAACUUGUUGGCGAGAUU
UU;
(vii)
(SEQ ID NO: 32)
tgggtgagattagaggccactgGUUUUAGUACUCUGGAAACAGAAUCUAC
UAAAACAAGGCAAAAUGCCGUGUUUAUCUCGUCAACUUGUUGGCGAGAUU
UU;
(viii)
(SEQ ID NO: 33)
tgcttcctcccttgtctccctaGUUUUAGUACUCUGGAAACAGAAUCUAC
UAAAACAAGGCAAAAUGCCGUGUUUAUCUCGUCAACUUGUUGGCGAGAUU
UU;
(iv)
(SEQ ID NO: 34)
tggcatatgagaaaagtcacagGUUUUAGUACUCUGGAAACAGAAUCUAC
UAAAACAAGGCAAAAUGCCGUGUUUAUCUCGUCAACUUGUUGGCGAGAUU
UU;
and
(x)
(SEQ ID NO: 35)
ccttattcttttgatatactccGUUUUAGUACUCUGGAAACAGAAUCUAC
UAAAACAAGGCAAAAUGCCGUGUUUAUCUCGUCAACUUGUUGGCGAGAUU
UU.

It is understood that because the gRNA sequences described above are RNA sequences. Any T (thymine) in the sequences referring to gRNAs would refer to U (or uracil) in the context of RNA molecules. Sequences containing T (thymine) herein would encompass both DNA molecules and RNA molecules (wherein T refers to U).

Moreover, the single-molecule gRNA can comprise no uracil at the 3′ end of the gRNA sequence. The gRNA can comprise one or more uracil at the 3′ end of the gRNA sequence. For example, the gRNA can comprise 1 uracil (U) at the 3′ end of the gRNA sequence. The gRNA can comprise 2 uracil (UU) at the 3′ end of the gRNA sequence. The gRNA can comprise 3 uracil (UUU) at the 3′ end of the gRNA sequence. The gRNA can comprise 4 uracil (UUUU) at the 3′ end of the gRNA sequence. The gRNA can comprise 5 uracil (UUUUU) at the 3′ end of the gRNA sequence. The gRNA can comprise 6 uracil (UUUUUU) at the 3′ end of the gRNA sequence. The gRNA can comprise 7 uracil (UUUUUUU) at the 3′ end of the gRNA sequence. The gRNA can comprise 8 uracil (UUUUUUUU) at the 3′ end of the gRNA sequence.

It is further understood that the nucleotides of the gRNAs described above may comprise modified nucleic acids at any nucleotide position. Accordingly, a gRNA can be unmodified or modified. For example, modified gRNAs can comprise one or more 2′-O-methyl phosphorothioate nucleotides. Examples of additional modified nucleic acids are known to those having skill in the art.

(iv) Ribonucleoprotein Complexes

In some instances, the gene-editing system disclosed herein may comprise a ribonucleoprotein complex (RNP), in which a gRNA and a nuclease (e.g., as described above) form a complex. As used herein, the term “ribonucleoprotein” or “RNP” refers to a protein that is structurally associated with a nucleic acid (either DNA or RNA). For example, in some embodiments, a Cas9 RNA-guided endonuclease and a gRNA of a gene-editing system are in the form of an RNP.

C. Donor Template

A donor template comprises a nucleic acid sequence that is to be inserted into a target site in a DNA sequence (e.g., in an endogenous gene). Accordingly, in some embodiments, a donor template of a gene-editing system may comprise a CFTR mini-gene containing exons 11 to 27 of a CFTR gene. The donor template may further comprise the nucleotide sequence of an acceptor splice site and/or one or more homologous arms.

A donor template of a gene-editing system may be provided in a synthesized form. Alternatively, a gene-editing system may comprise an agent (e.g., a nucleic acid such as a vector) for the production of a donor template. For example, a gene-editing system may comprise a nucleic acid (e.g., a vector) for producing the donor template.

(i) CFTR Mini-gene Comprising Exons 11 to 27 of CFTR

The donor template may comprise a CFTR mini-gene coding for exons 11 to 27 of a CFTR gene (e.g., hsCFTR). The CFTR mini-gene may contain one or more of intron 11 to intron 26 as in a wild-type CFTR gene. For example, the CFTR mini-gene may comprise an intron nucleotide sequence located 3′ to exon 11 of the CFTR gene and/or an intron nucleotide sequence between one or more of exons 11 and 12, exons 12 and 13, exons 13 and 14, exons 14 and 15, exons 15 and 16, exons 16 and 17, exons 17 and 18, exons 18 and 19, exons 19 and 20, exons 20 and 21, exons 21 and 22, exons 22 and 23, exons 23 and 24, exons 24 and 25, exons 25 and 26, and exons 26 and 27. In some instances, the CFTR mini-gene lacks at least one of introns 11-26.

If the CFTR mini-gene contains an intron nucleotide sequence between exon 11 and exon 27, such an intron sequence may be a native CFTR intron. For example, the intron 3′ to exon 11 of CFTR may be a wild-type CFTR intron 10. Likewise, wild-type CFTR intron 11, 12, 13, 14, 15, 16, 17, 18, 19, 20, 21, 22, 23, 24, 25, and/or 26 (or any combination thereof) may be positioned between one or more of exons 11 and 12, exons 12 and 13, exons 13 and 14, exons 14 and 15, exons 15 and 16, exons 16 and 17, exons 17 and 18, exons 18 and 19, exons 19 and 20, exons 20 and 21, exons 21 and 22, exons 22 and 23, exons 23 and 24, exons 24 and 25, exons 25 and 26, and/or exons 26 and 27 (or any combination thereof).

Alternatively, the CFTR mini-gene may contain a synthetic intron (i.e., non-endogenous to the CFTR gene) between exon 11-27. In some embodiments, the first nucleotide sequence comprises a synthetic intron between exons 11 and 12, exons 12 and 13, exons 13 and 14, exons 14 and 15, exons 15 and 16, exons 16 and 17, exons 17 and 18, 18 and 19, 19 and 20, 20 and 21, 21 and 22, 22 and 23, 23 and 24, 24 and 25, 25 and 26, and/or 26 and 27 (or any combination thereof). In some embodiments, a synthetic intron comprises a modified CFTR intron nucleotide sequence (i.e., a native CFTR intron that has been modified by addition or deletion of one or more nucleotides).

In some embodiments, the CFTR mini-gene may be free from any intron sequence (e.g., between exon 11-27). In some instances, the CFTR mini-gene encodes a fragment of a wild-type human CFTR, which is encoded by exon 11-27 of a wild-type human CFTR gene. For example, the CFTR mini-gene may comprise a nucleotide sequence encoding the following CFTR fragment (SEQ ID NO: 36):

MVIMGELEPSEGKIKHSGRISFCSQFSWIMPGTIKENIIFGVSYDEYRYR
SVIKACQLEEDISKFAEKDNIVLGEGGITLSGGQRARISLARAVYKDADL
YLLDSPFGYLDVLTEKEIFESCVCKLMANKTRILVTSKMEHLKKADKILI
LHEGSSYFYGTFSELQNLQPDFSSKLMGCDSFDQFSAERRNSILTETLHR
FSLEGDAPVSWTETKKQSFKQTGEFGEKRKNSILNPINSIRKFSIVQKTP
LQMNGIEEDSDEPLERRLSLVPDSEQGEAILPRISVISTGPTLQARRRQS
VLNLMTHSVNQGQNIHRKTTASTRKVSLAPQANLTELDIYSRRLSQETGL
EISEEINEEDLKECFFDDMESIPAVTTWNTYLRYITVHKSLIFVLIWCLV
IFLAEVAASLVVLWLLGNTPLQDKGNSTHSRNNSYAVIITSTSSYYVFYI
YVGVADTLLAMGFFRGLPLVHTLITVSKILHHKMLHSVLQAPMSTLNTLK
AGGILNRFSKDIAILDDLLPLTIFDFIQLLLIVIGAIAVVAVLQPYIFVA
TVPVIVAFIMLRAYFLQTSQQLKQLESEGRSPIFTHLVTSLKGLWTLRAF
GRQPYFETLFHKALNLHTANWFLYLSTLRWFQMRIEMIFVIFFIAVTFIS
ILTTGEGEGRVGIILTLAMNIMSTLQWAVNSSIDVDSLMRSVSRVFKFID
MPTEGKPTKSTKPYKNGQLSKVMIIENSHVKKDDIWPSGGQMTVKDLTAK
YTEGGNAILENISFSISPGQRVGLLGRTGSGKSTLLSAFLRLLNTEGEIQ
IDGVSWDSITLQQWRKAFGVIPQKVFIFSGTFRKNLDPYEQWSDQEIWKV
ADEVGLRSVIEQFPGKLDFVLVDGGCVLSHGHKQLMCLARSVLSKAKILL
LDEPSAHLDPVTYQIIRRTLKQAFADCTVILCEHRIEAMLECQQFLVIEE
NKVRQYDSIQKLLNERSLFRQAISPSDRVKLFPHRNSSKCKSKPQIAALK
EETEEEVQDTRL

In one example, the CFTR mini-gene comprises the nucleotide sequence of SEQ ID NO: 37, which encodes the above-noted CFTR fragment (SEQ ID NO: 36).

Alternatively, the CFTR mini-gene may comprise a nucleotide sequence that shares at least 60, at least 65, at least 70, at least 75, at least 80, at least 85, at least 90, or at least 95 percent identity with SEQ ID NO: 37 and encodes a functional CFTR fragment (e.g., SEQ ID NO: 36).

(ii) Acceptor Splice Site

A donor template may further comprise a nucleotide fragment located 5′ to the CFTR mini-gene to provide an acceptor splice site such that the 5′ end of the CFTR mini-gene may be connected accurately to the upstream exon 9 via RNA splicing after being inserted into intron 10 of the CFTR gene. In some examples, the nucleotide fragment carrying the splice acceptor site may be a 3′ end fragment of intron 10 of a native CFTR gene. In some embodiments, the acceptor splice site may be the native acceptor splice site of intron 10 of the native CFTR gene. In other embodiments, the acceptor splice site can be a synthetic (i.e., non-native) splice acceptor site. Examples of nucleotide sequences comprising synthetic acceptor splice sites are known to those having skill in the art. Any of the nucleotide fragment carrying a slice acceptor site may be of 50-200 nucleotides in length (e.g., 80-150 or 100-150 nucleotides in length).

For example, in some embodiments, the first fragment encodes for a synthetic acceptor splice site comprises, from 5′ to 3′: TATACACTTCTGCTTAGGATGATAATTGGAGGCAA GTGAATCCTGAGCGTGATTTGATAATGACCTAATAATGATGGGTTTTATTTCCAG (SEQ ID NO: 1), wherein the 3′ end AG is the slicing acceptor site.

(iii) Homologous Arms

The donor template may further comprise one or more homologous arms flanking the CFTR mini-gene to allow for efficient homology dependent recombination (HDR) at a genomic location of interest (e.g., intron 10 of CFTR). The length of a homologous arm may vary. For example, a homologous arm may be at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, or at least 1000 nucleotides in length. Likewise, a homologous arm may be 50 to 100, 50 to 200, 50 to 300, 50 to 400, 50 to 500, 50 to 600, 50 to 700, 50 to 800, 50 to 900, 50 to 1000, 100 to 200, 100 to 300, 100 to 400, 100 to 500, 100 to 600, 100 to 700, 100 to 800, 100 to 900, 100 to 1000, 200 to 300, 200 to 400, 200 to 500, 200 to 600, 200 to 700, 200 to 800, 200 to 900, 200 to 1000, 300 to 400, 300 to 500, 300 to 600, 300 to 700, 300 to 800, 300 to 900, 300 to 1000, 400 to 500, 400 to 600, 400 to 700, 400 to 800, 400 to 900, 400 to 1000, 500 to 600, 500 to 700, 500 to 800, 500 to 900, 500 to 1000, 600 to 700, 600 to 800, 600 to 900, 600 to 1000, 700 to 800, 700 to 900, 700 to 1000, 800 to 900, 800 to 1000, or 900 to 1000 nucleotides in length. In particular, a homologous arm may be 500 nucleotides in length.

For example, in some embodiments a donor template comprises a 5′ homologous arm (i.e., positioned upstream to the first nucleotide sequence) and a 3′ homologous arm (i.e., positioned downstream to the first nucleotide sequence), wherein the 5′ homologous arm comprises a nucleic acid sequence that is homologous to a region upstream to the genomic location of interest, and wherein the 3′ homologous arm comprises a nucleic acid sequence that is homologous to a region downstream to the genomic location of interest. In some embodiments, the 5′ homologous arm and the 3′ homologous arm comprise nucleotide sequences selected from the group consisting of:

(i) SEQ ID NO: 17 and SEQ ID NO: 18, respectively;

(ii) SEQ ID NO: 19 and SEQ ID NO: 20, respectively;

(iii) SEQ ID NO: 21 and SEQ ID NO: 22, respectively;

(iv) SEQ ID NO: 23 and SEQ ID NO: 24, respectively;

(v) SEQ ID NO: 25 and SEQ ID NO: 345, respectively;

(vi) SEQ ID NO: 346 and SEQ ID NO: 347, respectively;

(vii) SEQ ID NO: 348 and SEQ ID NO: 349, respectively;

(viii) SEQ ID NO: 350 and SEQ ID NO: 351, respectively;

(ix) SEQ ID NO: 352 and SEQ ID NO: 353, respectively; and

(x) SEQ ID NO: 354 and SEQ ID NO: 355, respectively.

In other embodiments, the donor template may comprise a 5′ homologous arm and lack a 3′ homologous arm. In yet other embodiments, the donor template may comprise a 3′ homologous arm and lack a 5′ homologous arm. A 5′ or 3′ homologous arm may comprise the nucleotide sequence of any one of SEQ ID NOs: 17-25 and SEQ ID NOs: 345-355, respectively.

Alternatively, a donor template may lack homologous arms. For example, in some instances, a donor template may be integrated by NHEJ-dependent end joining following cleavage at the target site.

In some instances, the donor template may comprise, from 5′ end to 3′ end, a 5′ homologous arm, a nucleotide fragment containing a splice acceptor site, a CFTR mini-gene, and a 3′ end homologous arm. These components may be linked directly. Alternatively, they may be linked via a nucleotide linker.

A donor template can be DNA or RNA, single-stranded and/or double-stranded, and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al., (1987) Proc. Natl. Acad. Sci. USA 84:4959-4963; Nehls et al., (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A donor template can be introduced into a cell as part of a vector molecule having additional sequences such as, for example, replication origins, promoters and genes encoding antibiotic resistance. Moreover, a donor template can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

A donor template, in some embodiments, is inserted so that its expression is driven by the endogenous promoter, such as the promoter that drives expression of the endogenous gene into which the donor is inserted.

Furthermore, exogenous sequences may also include transcriptional or translational regulatory sequences, for example, promoters, enhancers, insulators, internal ribosome entry sites, sequences encoding 2A peptides and/or polyadenylation signals.

D. Viral Vector/Viral Particle-Based Gene-Editing System

In some embodiments, the gene-editing system disclosed herein may comprise polynucleic acids (e.g., vectors such as viral vectors) or viral particles comprising such. The polynucleic acid(s) produces the components (e.g., a nuclease, a gRNA, and a donor template) for editing a CFTR gene as described herein.

In some examples, the gene-editing system comprises one polynucleic acid capable of producing all components of the gene-editing system, including a nuclease, a gRNA, and a donor template. In other examples, the gene-editing system comprises two polynucleic acids, one encoding the nuclease and the gRNA and the other comprising the donor template. Alternatively, the gene-editing system comprises two polynucleic acids, one encoding the nuclease and the other comprising the donor template and encoding the gRNA. In another example, the gene-editing system comprises three polynucleic acids, the first one encoding the nuclease, the second one encoding the gRNA, and the third one comprising the donor template.

The nucleic acid (or at least one nucleic acid in the set of nucleic acids) may be a vector such as a viral vector, such as a retroviral vector, an adenovirus vector, an adeno-associated viral (AAV) vector, and a herpes simplex virus (HSV) vector.

In some examples, the gene-editing system may comprise one or more viral particles that carry genetic materials for producing the components of the gene-editing system as disclosed herein. A viral particle (e.g., AAV particle) may comprise one or more components (or agents for producing one or more components) of a gene-editing system (e.g., as described herein). A viral particle (or virion) comprises a nucleic acid, which encodes the viral genome, and an outer shell of protein (i.e., a capsid). In some instances, a viral particle further comprises an envelope of lipids that surround the protein shell.

In some examples, a viral particle comprises a polynucleic acid capable of producing all components of the gene-editing system, including a nuclease, a gRNA, and a donor template. In other examples, a viral particle comprises a polynucleic acid capable of producing one or more components of the gene-editing system. For example a viral particle may comprise a polynucleic acid capable of producing the nuclease and the gRNA. Alternatively, a viral particle may comprise a polynucleic acid capable of producing the donor template and encoding the gRNA. In another example, a viral particle may comprise a polynucleic acid capable of producing only one of the nuclease, the gRNA, or the donor template.

The viral particles described herein may be derived from any viral particle known in the art including, but not limited to, a retroviral particle, an adenovirus particle, an adeno-associated viral (AAV) particle, or a herpes simplex virus (HSV) particle. In some embodiments, the viral particle is an AAV particle.

In some embodiments, a set of viral particles comprises more than one gene-editing system. In some embodiments, each viral particle in the set of viral particles is an AAV particle. In other embodiments, a set of viral particles comprises more than one type of viral particle (e.g., a retroviral particle, an adenovirus particle, an adeno-associated viral (AAV) particle, or a herpes simplex virus (HSV) particle).

E. Additional Exemplary Gene-Editing Systems

In addition, the gene-editing system disclosed herein may comprise a nuclease (e.g., a Cas9 enzyme) as disclosed herein. Such a gene-editing system may further comprise the gRNA, and the donor template. The nuclease and the gRNA, optionally in combination with the donor template, may form an RNP for delivery. Further, the gene-editing system may further comprise the gRNA and a polynucleic acid (e.g., a vector as those described herein) for producing the donor template. The nuclease and the gRNA may form an RNP complex. Alternative, the gene-editing system may further comprise one or more polynucleic acids for producing the gRNA and the donor template.

Alternatively, the gene-editing system disclosed herein may comprise an agent for produce the nuclease, for example, an expression vector such as a viral vector as disclosed herein capable of expressing the nuclease. Such a gene-editing system may further comprise the gRNA and/or the donor template, or agents for producing such.

Any other format of the gene-editing system comprising the components as disclosed herein for modifying the CFTR gene or agents producing such are within the scope of the present disclosure.

### II. Methods of Editing a Cystic Fibrosis Transmembrane Regulator (CFTR) Gene

In some aspects, the disclosure relates to methods of editing a cystic fibrosis transmembrane regulator (CFTR) gene using any of the gene-editing systems disclosed herein. An editing event may correct a mutation in a CFTR gene. One or more copies (i.e., alleles) of a gene (e.g., CFTR) may comprise a mutation. In some embodiments, the methods of gene editing described herein may be used to correct at least one copy (i.e., allele) of a CFTR gene. In some embodiments, two copies (i.e., alleles) of a CFTR gene are edited.

More than 2000 mutations in CFTR have been described that confer a range of molecular and functional phenotypes. See e.g., Veit G. et al., Mol. Biol. Cell. 2016 Feb. 1; 27(3): 424-33. Examples include, but are not limited to, M1V, A46D, E56K, P67L, R74W, G85E, E92K, P99L, D110H, D110E, R117C, R117H, R170G, G178R, E193K, P205S, L206W, V232D, R334W, R334W, I336K, T338I, S341P, R347P, R347H, R352Q, L467P, S492F, ΔI507, ΔF508, V520F, G542X, S549R, S549N, G551S, G551N, G551D, A455E, S549N, R553X, A559T, R560T, R560S, R560K, A561E, Y569D, D579G, D614G, R668C, L927P, S945L, S977F, L997F, F1052V, H1054D, K1060T, L1065P, R1066C, R1066M, R1066H, A1067T, R1070Q, R1070W, F1074L, H1085R, M1101K, D1270N, D1152H, L1077P, S1235R, G1244E, S1251N, S1255P, W1282X, N1303K, G1349D, Q1411X, 2789+5G>A, and 3849+10kbC>T. In some embodiments, the methods described herein may be used to correct one or more mutation(s) listed above or otherwise known in the prior art.

A method of editing a CFTR gene may comprise contacting a cell with: a gene-editing system as described herein; a viral particle or set of viral particle comprising a gene-editing system as described herein; and/or a nucleic acid or set of nucleic acids comprising a gene-editing system as described herein. These methods may be performed, for example, on one or more cells existing within a living subject (e.g., in vivo). Alternatively or in addition, these methods may be performed on one or more cells existing in culture (e.g., in vitro). In some instances, a cell edited in culture is then administered to a subject (categorized herein as “cell-based therapy”).

A. Delivery Methods

The contacting of the cell (or subject) with the gene-editing system, viral particle or set of viral particles, and/or nucleic acid or set of nucleic acids may be performed via various delivery methods. For example, nucleases and/or donor templates may be delivered using a vector system, including, but not limited to, plasmid vectors, DNA minicircles, retroviral vectors, lentiviral vectors, adenovirus vectors, poxvirus vectors; herpesvirus vectors and adeno-associated virus vectors, and combinations thereof.

Conventional viral and non-viral based gene transfer methods can be used to introduce nucleic acids encoding nucleases and donor templates in cells. Non-viral vector delivery systems include DNA plasmids, DNA minicircles, naked nucleic acid, and nucleic acid complexed with a delivery vehicle such as a liposome or poloxamer. Viral vector delivery systems include DNA and RNA viruses, which have either episomal or integrated genomes after delivery to the cell.

Methods of non-viral delivery of nucleic acids include, but are not limited to, electroporation, lipofection, microinjection, biolistics, virosomes, liposomes, immunoliposomes, polycation or lipid:nucleic acid conjugates, naked DNA, naked RNA, capped RNA, artificial virions, and agent-enhanced uptake of DNA. Sonoporation using, e.g., the Sonitron 2000 system (Rich-Mar) can also be used for delivery of nucleic acids.

Methods for delivery of proteins (e.g., RNA-guided endonucleases) include, but are not limited to, the use of cell-penetrating peptides and nanovehicles.

(i) Adeno-Associated Viral Delivery

The donor nucleic acid encoding exons 11 to 27 of the CFTR gene may be delivered to a cell using an adeno-associated virus (AAV). AAVs are small viruses which integrate site-specifically into the host genome and can therefore deliver a transgene, such as exons 11 to 27 of the CFTR gene. Inverted terminal repeats (ITRs) are present flanking the AAV genome and/or the transgene of interest and serve as origins of replication. Also present in the AAV genome are rep and cap proteins which, when transcribed, form capsids which encapsulate the AAV genome for delivery into target cells. Surface receptors on these capsids which confer AAV serotype, which determines which target organs the capsids will primarily bind and thus what cells the AAV will most efficiently infect. There are twelve currently known human AAV serotypes. In some embodiments, the AAV is AAV serotype 6 (AAV6).

Adeno-associated viruses are among the most frequently used viruses for gene therapy for several reasons. First, AAVs do not provoke an immune response upon administration to mammals, including humans. Second, AAVs are effectively delivered to target cells, particularly when consideration is given to selecting the appropriate AAV serotype. Finally, AAVs have the ability to infect both dividing and non-dividing cells because the genome can persist in the host cell without integration. This trait makes them an ideal candidate for gene therapy.

(ii) Homology-Directed Repair (HDR)

The donor nucleic acid encoding exons 11 to 27 of the CFTR gene may be inserted into the target genomic region of the edited cell by homology directed repair (HDR). Both strands of the DNA at the target genomic region are cut by a CRISPR Cas9 enzyme. HDR then occurs to repair the double-strand break (DSB) and insert the donor DNA. For this to occur correctly, the donor sequence is designed with flanking residues which are complementary to the sequence surrounding the DSB site in the target gene (hereinafter “homology arms”). These homology arms serve as the template for DSB repair and allow HDR to be an essentially error-free mechanism. The rate of homology directed repair (HDR) is a function of the distance between the mutation and the cut site so choosing overlapping or nearby target sites is important. Templates can include extra sequences flanked by the homologous regions or can contain a sequence that differs from the genomic sequence, thus allowing sequence editing.

(iii) Non-Homologous End Joining (NHEJ)

The NHEJ pathway may also produce, at very low frequency, inserts containing exons 11-27. Such repair should correct CFTR expression when the insert is in the sense strand orientation.

B. Cell Therapy

The methods described herein may be performed on one or more cells existing in culture (e.g., in vitro). Accordingly, in some aspects, the disclosure relates to genetically edited cells comprising an edited cystic fibrosis transmembrane regulator (CFTR) gene in which an exogenous nucleic acid is inserted into intron 10 of the endogenous CFTR gene. The exogenous sequence may comprise one or more of the components of the donor template described above (e.g., a nucleotide sequence encoding exons 11 to 27 of CFTR, a nucleotide sequence encoding an acceptor splice site, and/or a nucleotide sequence encoding one or more homologous arm).

(i) Cells with Edited CFTR Gene

Genetically-edited cells may be produced using any of the methods described herein. In some embodiments, one or more gene edits within a population of edited cells results in a phenotype associated with changes in CFTR functionality.

In some embodiments, genetically-edited cells of the present disclosure exhibit increased CFTR activity (e.g., by at least 30%, 50%, 100%, 2-fold, 5-fold, or 10-fold) relative to the unedited control. For example, the levels of CFTR activity may be increased by at least 30%, at least 50%, at least 100%, at least 200%, at least 500%, at least 1000% relative to control unedited cells. In some embodiments, the levels of CFTR activity may be increased by 30%-50%, 30%-100%, 30%-200%, 30%-500%, 30%-1000%, 50%-100%, 50%-200%, 50%-500%, 50%-1000%, 100%-200%, 100%-500%, 100%-1000%, 200%-500%, 200%-1000%, or 500%-1000% relative to control T cells.

(ii) Methods of Administration

In some instances, a genetically edited cell may be administered to a subject. The step of administering may include the placement (e.g., transplantation) of genetically engineered cells into a subject, by a method or route that results in at least partial localization of the introduced cells at a desired site, such that a desired effect(s) is produced and where at least a portion of the implanted cells or components of the cells remain viable. The period of viability of the cells after administration to a subject can be as short as a few hours, e.g., twenty-four hours, to a few days, to as long as several years, or even the life time of the subject, i.e., long-term engraftment. In some embodiments, the administration is to the respiratory tract of the subject.

Modes of administration include injection, infusion, instillation, or ingestion. Injection includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the route is intravenous.

In some embodiments, genetically engineered cells are administered systemically, which refers to the administration of a population of cells other than directly into a target site, tissue, or organ, such that it enters, instead, the subject's circulatory system and, thus, is subject to metabolism and other like processes.

For use in the various aspects described herein, an effective amount of genetically engineered cells comprises at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. In some examples described herein, the cells are expanded in culture prior to administration to a subject in need thereof.

C. Effective Amount

In some aspects, the disclosure relates to methods of administering an effective amount of a gene-editing system as descried herein, a viral particle or set of viral particles comprising a gene-editing system as described herein, a nucleic acid or set of nucleic acids comprising a gene-editing system as described herein, or a composition of edited cells as described herein to a subject in need thereof.

A subject may be any subject for whom diagnosis, treatment, or therapy is desired. In some embodiments, the subject is a mammal. In some embodiments, the subject is a human. In some embodiments, the subject is a human patient having cystic fibrosis. In some embodiments, the human patient is a child.

An effective amount refers to the amount of a gene-editing system, a viral particle or set of viral particles comprising a gene-editing system, a nucleic acid or set of nucleic acids comprising a gene-editing system, or a population of genetically engineered cells needed to prevent or alleviate at least one or more signs or symptoms of a medical condition (i.e., CF), and relates to a sufficient amount of a composition to provide the desired effect (i.e., to treat a subject having CF). An effective amount also includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

The efficacy of a treatment comprising a composition for the treatment of a medical condition can be determined by the skilled clinician. A treatment is considered an “effective treatment,” if any one or all of the signs or symptoms of, as but one example, levels of functional target are altered in a beneficial manner (e.g., increased by at least 10%), or other clinically accepted symptoms or markers of disease (e.g., CF) are improved or ameliorated. Efficacy can also be measured by failure of a subject to worsen as assessed by hospitalization or need for medical interventions (e.g., progression of the disease is halted or at least slowed). Methods of measuring these indicators are known to those of skill in the art and/or described herein. Treatment includes any treatment of a disease in subject and includes: (1) inhibiting the disease, e.g., arresting, or slowing the progression of symptoms; or (2) relieving the disease, e.g., causing regression of symptoms; and (3) preventing or reducing the likelihood of the development of symptoms.

### III. Kits for Therapeutic Use

The present disclosure also provides kits for use of the compositions described herein. For example, the present disclosure provides kits comprising a gene-editing system as described herein; a viral particle or set of viral particle comprising a gene-editing system as described herein; a nucleic acid or set of nucleic acids comprising a gene-editing system as described herein; and/or a population of genetically-edited cells as described herein.

In some embodiments, the kit can additionally comprise instructions for use in any of the methods described herein. The included instructions may comprise a description of: (i) the delivery of a gene-editing system as described herein; a viral particle or set of viral particle comprising a gene-editing system as described herein; and/or a nucleic acid or set of nucleic acids comprising a gene-editing system as described herein; and/or (ii) the administration of a population of genetically-edited cells as described herein.

The kit may further comprise a description of selecting a subject suitable for treatment based on identifying whether the subject is in need of the treatment. The instructions may include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### IV. General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1. Gene-Editing Systems for Genetic Modification of a CFTR Gene

Methods

Cells and Culture: Lung Progenitor Cells (LPCs) were derived from human lung donors diagnosed with Cystic fibrosis. LPC donor ID numbers 14071 and 14335 contain the CFTR genotype dF508/dF508 and dF508/G542X, respectively. LPCs were derived and expanded using BEGM™ Bronchial Epithelial Cell Growth Medium (Lonza) supplemented with Vertex's proprietary reagents.

Human Bronchial Epithelial cells (HBEs) were obtained by directed differentiation of LPCs using the Air Liquid Interface (ALI) culture format. 80,000 LPC cells were seeded per well in the apical side of an ALI-96-well plate. LPC were fed from the apical and basolateral side every other day during 5 days by using BEGM™ Bronchial Epithelial Cell Growth Medium (Lonza) supplemented with Vertex's proprietary reagents. At day 6, apical media was removed to promote Air Liquid Interface. Basolateral media was replaced with Vertex's proprietary HBE differentiation media. Cells were fed every other day by replacing HBE differentiation media in the basolateral side. Complete HBE differentiation was obtained after 5 weeks of HBE differentiation.

CRISPR-Cas9 Gene-Editing Reagents: Synthetic gRNAs were purchased from Synthego. gRNAs were HPLC (high-performance liquid chromatography) purified and contained chemically modified nucleotides (2′-O-methyl 3′-phosphorothioate) at the three terminal positions at both the 5′ and 3′ ends. gRNAs contained a 22 nucleotide long spacer sequence to specifically target intron 10 of CFTR gene (see TABLE 1) and an 80 nucleotide long scaffold sequence that allows binding to saCAS9 protein.

saCas9 mRNA was design by CRISPR Therapeutics and synthetized by TriLink Biotechnologies. saCas9 mRNA expresses a Staphylococcus aureus Cas9 (Uniprot entry code J7RUA5) with SV40 and NucleoPlasmine nuclear localization signals. saCas9 mRNA also contains a CAP1 structure and a polyadenylated signal to obtain optimal expression levels in mammalian cells. saCAS9 mRNA was HPLC purified.

hsCFTR Intron 10
(SEQ ID NO: 38)
GTAGTTCTTTTGTTCTTCACTATTAAGAACTTAATTTGGTGTCCATGTCTCTTTTTTTTTCTAGTTTGT
AGTGCTGGAAGGTATTTTTGGAGAAATTCTTACATGAGCATTAGGAGAATGTATGGGTGTAGTGTCTTG
TATAATAGAAATTGTTCCACTGATAATTTACTCTAGTTTTTTATTTCCTCATATTATTTTCAGTGGCTT
TTTCTTCCACATCTTTATATTTTGCACCACATTCAACACTGTATCTTGCACATGGCGAGCATTCAATAA
CTTTATTGAATAAACAAATCATCCATTTTATCCATTCTTAACCAGAACAGACATTTTTTCAGAGCTGGT
CCAGGAAAATCATGACTTACATTTTGCCTTAGTAACCACATAAACAAAAGGTCTCCATTTTTGTTAACA
TTACAATTTTCAGAATAGATTTAGATTTGCTTATGATATATTATAAGGAAAAATTATTTAGTGGGATAG
TTTTTTGAGGAAATACATAGGAATGTTAATTTATTCAGTGGTCATCCTCTTCTCCATATCCCACCCTAA
GAACAACTTAACCTGGCATATTTGGAGATACATCTGAAAAAATAGTAGATTAGAAAGAAAAAACAGCAA
AAGGACCAAAACTTTATTGTCAGGAGAAGACTTTGTAGTGATCTTCAAGAATATAACCCATTGTGTAGA
TAATGGTAAAAACTTGCTCTCTTTTAACTATTGAGGAAATAAATTTAAAGACATGAAAGAATCAAATTA
GAGATGAGAAAGAGCTTTCTAGTATTAGAATGGGCTAAAGGGCAATAGGTATTTGCTTCAGAAGTCTAT
AAAATGGTTCCTTGTTCCCATTTGATTGTCATTTTAGCTGTGGTACTTTGTAGAAATGTGAGAAAAAGT
TTAGTGGTCTCTTGAAGCTTTTCAAAATACTTTCTAGAATTATACCGAATAATCTAAGACAAACAGAAA
AAGAAAGAGAGGAAGGAAGAAAGAAGGAAATGAGGAAGAAAGGAAGTAGGAGGAAGGAAGGAAGGAAAG
AAGGAAGGAAGTAAGAGGGAAGCAGTGCTGCTGCTGTAGGTAAAAATGTTAATGAAAATAGAAATTAAG
AAAGACTCCTGAAAGGCAATTATTTATCAATATCTAAGATGAGGAGAACCATATTTTGAAGAATTGAAT
ATGAGACTTGGGAAACAAAATGCCACAAAAAATTTCCACTCAATAAATTTGGTGTCAGGCTGGGTGCAG
TGGCTCACACTTGTAATCCTAGCACTTTTGGAGGCAGAGGCAGGTGAATTGCTTGAGTCCAGGAGTTTG
AGACCAGCGTGGGCAACATGGCAAACCCCACCTCTACAAAAAACACAAACAAAAGAAAATAGCTGGGTG
TGGTGGTGTGTGCCTGTAGTCCCAGCTACTTGGGAGGCTGAGGTGGGAGGATCACCTGAGCCTGAGAAG
TGGAGGCTGCAGTGAGCCATGATTGCACCACTGTACCCTAGCCTAGGTGATAGGCTCAAAAAAAAAAAA
AATTGGTGTTTGCAATGCTAATAATACAATTTGGTTGTTTCTCTCTCCAGTTGTTTTCCTACATACGAA
ACAGCTTTTAAAACAAAATAGCTGGAATTGTGCATTTTTTCTTACAAAAACATTTTCTTTCTTAAAATG
TTATTATTTTTCTTTTATATCTTGTATATTATTACTAGCAGTGTTCACTATTAAAAAATTATACTATAG
GAGGGGCTGATACTAAATAAGTTAGCAATGGTCTAAACAAGGATGTTTATTTATGAAAAGGTAGTAATT
GTGTTTCATAGAATTTTTAAAATTAATTCTGCGTATGTCTTCAAGATCAATTCTATGATAGATGTGCAA
AAATAGCTTTGGAATTACAAATTCCAAGACTTACTGGCAATTAAATTTCAGGCAGTTTTATTAAAATTG
ATGAGCAGATAATTACTGGCTGACAGTGCAGTTATAGCTTATGAAAAGCAGCTATGAAGGCAGAGTTAG
AGGAAGGCAGTGGTCCCTTGGGAATATTTAAACACTTCTGAGAAACGGAGTTTACTAACTCAATCTAGG
AGGCTGCCTTTTAGTAGTATTAGGAATGGAACACTTTATAGTTTTTTTTGGACAAAAGATCTAGCTAAA
ATATAAGATTGAATAATTGAAAATATTAACATTTTAAGTTAAATCTTACCCACTCAATACAATTTGGTA
ATTTGTATCAGAAGCTTAAAAGATAACCTAATAGTTCTTCTACTTCTATAACTTACCCAAATATGTTTG
CAGAGATCTTATGTAAAGCTCTTCATTATAACACTGCTTTCAGGAGCCAAAAATTGGGTGGGGGAGCCC
CATAAATGTTGAATAATAGGGGTTTGATTAGATAAATTTTGGTGTAGTTCTATAATGGCGTGTTATTCA
GCCAATAAAAGGTTTGTTAAAGAATGACTGTGACGGATGTATATGATATACTCTTAAGTGAATAAAGAG
TTACAAAATGTTATGTACAAGTTACAAAATGTATGTACATTATGATCCATTTTTCATAAAATCATATGT
ATGTATATATGTGTGTCTGGAAGGATAAATTTATCAAGTTGTTATCTCTGAAATTTTGGGTATATTTTA
TATTTCTAGATTTTCTGTTACTTTGTTACTTTACTGATAAAGTAATAACGTTGTTGACTTTTGTCACTC
TCCCCTATTAATAATCATCTAGGCTGCAAAAGGATCATGTCTTCTTTATTTTTATATTCCAAGGACTGT
CAACAAGTGCCTAGCACTTGACAGGTATATTATAGAAATTTAACTGAATATCTTTAGGAAATAGATTTT
TGTTTGTAGTTGTTCTAGTCTACATTAAATGTCTTGCGCTTATGAAACTTCCTTGAATTATTTTAGTGA
AGCAATATTAGTATAGAATTTTGCATCACTGGATGCCCTTGACTGAAAGCTGGCTTATGGCATCTCACC
AGTGTGTGGGGAGTTTCAGTCCTTCTGTTGTCTGCATCACAGCTGAAGCAGTGCTGTTGCTGACAATTC
CTGACACCACCTTGTCTCTATTATTGATCATTGCCTCACTATGGTACTGAGTTTTAGCTTATTCTTGTA
ATAACTGGGACTCATATGTATAGAATAAGCTATTAGCTCACGTTTTTGCTTGCTTTTTATACAGAATAC
ATGTCTGCAAATAGTTTTATCAATATTTTGGAATTTTGGGAGATATGAAGTTAAAAACATCATTGAATA
TATATATATACACACACACATATATATATGACACTATACATGATTTATTTTATTTAATTTTTAAAATTT
TATTCTTTTTAGAGATTAGGTCTTACTCTGTCACCCAGGCTGAACTTCAGTGGTGTGATCATAGCTCAC
TGTAACCTTGAACTCCTGGGCTCAATTGACCTTTCCGCTTCAGCCTCCCAAAGTGCTGGGTTTATAGGC
ATGAGCCACTGTGTCTGGTCCAATATGCATATATATATTTTTAACCTGGATTATCAGAGCTATATTGTG
TTTAGGTTTATAAAGCTGTACTATGTGAAAATATCACTTCTAGGTTTAATTTTGTACAAAGGAATTTTA
TATAGAAATGAGGTAATTCAGATTTTTTCCCATGTAATAAGAATTGTAAAATTTACTGAAACAAACATC
AAAAAGATATCTGTTACATGACCTTCCTTTCTTTTGAATATATTTCAGGTGATATTATTTATTAAAATT
TAAAAATGAAAATTAAAATATATAAAAAGTTGAAAATTATTCCTTTCTTTACTGTCTCTCATCTGTCCA
TTTTCCATTCTCCTGCATTCCCTCATCCAACCAAGGTAGCCAATCCAGGTAACTTTTTTTAGTATCTTC
CCAGAGATGTTTCTCTCTATATATATAATCAATATACATTTTTTATTATTCCCCACCTCTCTTTTTATG
TAACAATATGCAGAGTTTTGCTTCTTGCTTTTCCCACTATCTTGGACAACTTTCCATATTCAAAGCACA
GAGGACTTGCACATATGTTCAGACTGCTGAATATTTCTGTCTCTCCCCTGCCATTCATATGTTGAAATC
CTAATTCCCAAGGTGATGGTATTGCAGGGTGGGGCCTTTGGGAGGTGATTAGTCCATGAGGGTGAAGTC
TTTAGTAAATGAGATTAGTGTCTTTATAAAAGAAACCTTAGAGAGACCCTCACACCTTAGAGAGACCCT
CACCCCTTTCTGCCATGTGAGAACACAGCAGGAAGACAGCTGGCTATCCAGGATTCAGGAGTCTCTTAG
CAGACCCAAATCTGCTGGCACCTTGATCTTGGACTTCCCAGCCTCCAGAACTGTGAGAAATAAATTCCT
GTTGTTTATAAGCCACACAGTTCATGGTATTTTGTTATAGCAGCCTGAACAAGGACACACACACACACA
CACACACATGCACACACATTTAAATAGATGCATAGTATTCTATCATATGGATGGATATTCTATGATATA
ATGAATCACTATTGATTGACATTTGGGTTGTTTCCAATATTTTGTTAACACAAAGAACAACACTACAAA
TAACTTTATATACATATCATTTAGCACATCTGCAATTGTATCAGTAGGCTTCCTATAAGTGGTCAAGCA
TTTGTGTACTTGTGATTTTGGTAGATGTTGTCAAATGTCCTTCCCTGAAATTTGTACCAATTCGTACTC
ATGCCATACACTCTAAATAGAGTGCTGATTTCCCCACAGCATTACTAACAGATGATATTATCTAATTTA
AAAAGTTTCTCATCTTATAGGGAAAATAGTATGTCAATGTATTCTTAACTTGCATTTCTTTTATTATAA
GTAGTGTAAAATATCATTTCAACTTATACACAGGAGGAATTTCTCTCTATATAAAGTGATCCTAGAATC
ATAATGAAAAATATCACCAACTCATTAGGAAAATGTACAAAGGATTGAATAGATATCTCATCAAAAATA
AAAATATAAGTGGCCTTTAAACATTGAAAGGTAACATTTGAACAAAGACTTGCAGGAGGTGAGGGATTA
GGGAATGCAGACTCTGGGAAGAGTCTTCCAAGTAGCAGGTGAAGCAAGTGCAAAGCTTTCAGATGGGAC
TGACTATACCTGTCTGGTTTGAAGAACAGTAAGGAGGTCACTGAGGCTGGCATAGAGTAAGACAGGGAG
GGTAGAATACTGTCAGAGAAGTAATCGGCGGTGGAGGTAGGGGGTAAACCATAAAGTGCTCGTAAAGAC
TAAGGCTTATTTCTCTGGGTGAGATTAGAGGCCACTGGAGAGTTTTAAACAGAAGTAACAGGGCCACTT
TGGCTAATGTTTTTAGGCTATTCTGTAGGGAGACAAGGGAGGAAGCAAGGAGATGAGTTAGGAGTCTAT
TGTGCCAGTTCAGGCAAGTGATGATGGTGGCTTGATCCAGGTAGTAGTGGAAGTAGTATAGTAGGAAGT
GATCAGATTCAGGACATGCTTTGAAGGAAGATCCAATAGGATTAATGGATAAGTTGAACAATGGCATAT
GAGAAAAGTCACAGAGGAGTCAAAGATGATTCCAAGCTTTCTGGACTGAGTAACTGGAAGGATAAATGT
GCCGTTTACTAGAAAGATAATGGGAGAAACAGGTTTTGGATGGAGCTTGGTTTGGGAATATTAAGTTTG
AAATGCCTATTTGACATCCAAATAGAGATGTTAGTTGGATGTACAAGTCTAGTTTCAAGGAAGAGGGGG
CTGGTAGTGTGAAGATGGGGCTGGATAAGATTCTAAAGGAAAGAGGGTTGATAAGAAGAGAAAGGGGTG
TAGGGGTTAGCCTAAGGGCATTCTAAGTATTAGAGGTTAAGGAGGTGGGTGAAGAAAACCCAATAAAAT
AAAAGTCTGAGAAGACAAAGCTAGTGAATGAATGTGGTATCCCGGAACCCAACTGATGTCAAGCAGAAG
GGTGTTATCAACTAGGTCAAATGCTCATTCATCAAGTAAGATGAAACTGTTATAATTAACCGGTGTCTT
CTGAAATACGGAGATAACTCGTGACTTAATGAAAGCAATAGTAGAGAAGGTCAAACTTGACCAGAATGA
AATTAGAAAGAATAAGAGGAAAGAAAAGACCAAATACAGACAACCATTGATGCCTTATTCTTTTGATAT
ACTCCTGGAGTCCACTTGCTAATACAATTGACCCTTAAACAATACAGGCTTGAACTGCATGGGTCCACT
TATTTGTGAATTTTTTTTCAGTTAATACATTGGAAAATTTTTGGGGTTTTTTGACAATTTGAAAAAACT
CACAAACTGTCTAGCCTAGAAATACCGAGAAAATTAAGAAAAAGTAAGATATGCCATGAATGCATAAAA
TATATGTAGACACTAGCCTATTTTATCATTTGCTACTATAAAATATACACAATCTATTATAAAAAGTTA
AAATTTATCAAAACTTAACACACACTAACACCTACCCTACCTGGCACCATTCACAGTAAAGAGAAATGT
AAATAAACATAAAAATGTAGTATTAAACCATAATGGCATAAAACTAATTGTAGTACATATGGTACTACT
GTAATAATTTGGAAGCCACTTCCTGTTGCTATTACGGTAAGCTCAAGCATTGTGGATAGCCATTTAAAA
CACCACGTGATGCTAATCATCTCCGTGTGAGCAGTTCTCTCTCCAGTAAATTGCATATTGCAGTAAAAA
GTGATCTCTAGTGGTTCTCGCATATTTTTCATCATGTTTAGTGCAATGCCATAAACCTTGAATAACATC
AAGCAATCCATACAAAGTGCCACTAGTGATGCACGGAAAAGTTGTAACAGTACAAGAAAAAAGTTGAGT
TGCTTGGTATTTACCATATATTGAGGTCTGCAGCTACAGTTGCCTGCAATTTCGAGATAAATGAACCCA
GTATAAAGACTGTTGTAACAAAAGAAAAGAAAATGTGAAACCATCAGTGCAGCTATGCCAGCAGGTGTG
AAGTCTTGCACTTTTTGCAAAATACAAAATATGAAATATGTGTTAATTGACTGTTTATGTTATCTGTAA
GGTTTCCACTCAACAATAGGCTATTAGTAGTTAAGTTTTTGTGGAGTCAAAAATTATACGTGGATTTTT
GACTATACAGTGGGTTGGCACCCCTAACCTTCATGTTGATAAAGGGTCAATGGTATATTATTTAATTTT
TTTGTATTTATATTCATAAATAAGATTAAATCTATATTTCCAAGTAATCTCTATAAGATTTTGTTATTA
ATATTACTATTATTTTTGAGACAGAGTCTTACTGTCACCAGGCTGGAGCACAGTGGTGCGATCTCGGCT
CACTGCAACCTCTGCCTCCCGGGCTCAAGCAATTCTCCTGCCTCACCCTCCCAAGTAGCTGGGACTACA
GGCACGCACAACCACACTCAGCTAATTTTTGTATTTTTAGTAGAGACGGGGTTTCACCATGTTGGCCAG
GATGGTATTGATCTCTTGACCTCATGATCTGCCTGCCTCGGCCTCCCAAAGTGTTGGGATTACAGGCAT
GAGCCACTGTGCACAGCCATTAATATTATTGTTACCCAATAAAAAAAATTTGGAAACTTGTCTTCTTTT
CCCCTGATTCTGTTTAAATAGCACTGGAGTTACCTGTTTTGAATTTTTTTTCCAAGCGGTCCCTTATGA
GTTTTCTCTATGTTTTATTTGTTTCATTTCTTTTTTTTTTTTTTTTTTTTTTTTTGAGACGGAGTCTCG
CTCTGTCGCCCAGGCTGGAGTGCAGTGGCGGGATCTCGGCTCACTGCAAGCTCCGCCTCCCGGGTTCAC
GCCATTCTCCTGCCTCAGCCTCCCAAGTAGCTGGGACTACAGGCGCCCGCCACTACGCCCGGCTAATTT
TTTGTATTTTTAGTAGAGACGGGGTTTCACCGTTTTAGCCGGGATGGTCTCGATCTCCTGACCTCGTGA
TCCGCCCGCCTCGGCCTCCCAAAGTGCTGGGATTACAGGCGTGAGCCACCGCGCCCGGCCTGTTTCATT
TCTTATATCGTATTTTTGCAACTCCTTTATTGATACTTTTCTTCCTGATTAGGTTTCTACTAAAACCAA
ACAAGCTTTCCATGAATTAGCTTTTAGATTTACTTATTAGTTTAACTGTTCTGTTGTATTGTAACTCAT
TAATTTATAATTTTATCTTTATTAATTATTCTATTTTTCTTCGCTTTTTTGTTGTTTTTCTAGTTTTTG
AGTTAGATGTTTGACGCTTTTTTAAAAAGCTGTGCATTTTCCTCTGGGTAATACTTTAGCTGTATATTA
TGTATTCTGATATATAGTGTTTCCATTACATTGTTTTCTAGAAAATCTGTAGCTTTGATTTATATTTGT
TTCCTCTTTGACCTAAGATATCCTAAGGGAAAATTTAACATTTTCCAGAAAGAAAACAAATTTTCTTTG
TTTTCCAAGAATGTTGTTCAAATTATTTCTACTGCTTGGAATTTTTATCATTTTTGTGTATCCAGTAAA
TAGTCAATATTTGTACTTGCTCTCTGACCACATAAAAGAATATATTCGTGTAGTTTCTATTAATAGATT
AGAGTTCAATTCAGATATTAAATGTACATCATTATTCATGATATTTAGGTCTTCTACATCTTCACTTAT
CTTTTTTCTACTTGCTTTGCCATTAACAGATAAAGTTGAATTAAAGGCTTCTACTACATACATTTCTCC
CTGTTATTCCTTATAGGTTCTGTAATTTTTGCTTCAAGAATATTGCTTTTTAAATTTAATATATAGATA
CTTATAATTACACTCTAGCATTATAAAGAGCCTTTTCTTTTTCATTGAATGTATTTGGGCCTGCATATG
TCTAACATGAAAATTATAGTCCTTTTTTTGTTTCTTTGTTTGTATTTACAGTTTTAAGTTCCATTTTCA
ACCTTTATGCACTCTTTGCTTTAGGTGTGTCTCTTTTAGTTAGCATAAAGTTAGGTTTGTCTTTAATTT
CACCTGAAGTCTTTTCCTCTTAATAGATGGGTTAAGCCAACTGAAAAATAAAACTGACTTATATACTTT
TATTTCAAGTATGTCCTCCACAAATATTTTTTGAATAGATTAGCTTATATACTTTGGAATTTGTTAAAA
AAAGATTTTTATAAAAAATAATTGTGGTGAAATGTACATAACATAAAATTTATCATTTTGACCATTTTT
AAGGGCATAGCTCTGTGGCATAAAGTATACTCACATAGTTGTGCAACTATCACCTCCTTTTGATTTTTT
TTTACTAATTTTGTAAATTTGTTTCATCTGAGCTGTCTTATTATGTTTTGTTTTATGTTTTTCTTTCCT
TTATTATGAAGTCACTGTATTGTCTGTAGGCTATATGTATCTGTGAGTGTGTGTGTATATGTGTGTATT
ATGGTTTTTAAAAAAGTCTATATTTGTTTTCCAGTGGCTATACTTAATACTAATAACTTTATGTTAAAT
TTTTCATTCTATGTGACTCTAGTTCACTAATATGAGCTCTGATAAAATCAGTGCTTTTTCGAGGTTAGG
AGATCAAGACCATCCTGGCTAACACAGTGAAACTCCGTCTCTACTAAAAATACAAAAAATTAGCCAGAC
GTGATGGCGGGTGCCCGTAGTCCCAGCTACTCGGGAGGCTGAGGCAGGAGAATGGCGTGAACCCAGGAG
GCAGAACTTGCAGTGAGCCGAGATCGCGCCACTGCACTCTAGCCTGGGTGACAGAGTGAGACTCTGTCT
CTAAATAAATAAATAAATAAATAAATAAATAAATAAAATCAGTGCTTTTTCTTCCTCTGCTACCTCCTT
TCCTTCTACTCAGTTTTAGTCAGTAGTATTATCTTTTTTCAGATTTATCTTTGTATTGTTAAATCTGCT
TATGCTTCTATTACTTTATTTATTAGCTTTAAATGATACCTTTTGACTTTCAGCTTTTCTTAATAAAGC
AATCAGCAAATTTCCTTTACACTCCACACTTATACCCCATTTCCTTTGTTTGTTTATTTGGTTTTTACT
TCTAACTTTTCTTATTGTCAGGACATATAACATATTTAAACTTTGTTTTTCAACTCGAATTCTGCCATT
AGTTTTAATTTTTGTTCACAGTTATATAAATCTTTGTTCACTGATAGTCCTTTTGTACTATCATCTCTT
AAATGACTTTATACTCCAAGAAAGGCTCATGGGAACAATATTACCTGAATATGTCTCTATTACTTAATC
TGTACCTAATAATATGAAGGTAATCTACTTTGTAGGATTTCTGTGAAGATTAAATAAATTAATATAGTT
AAAGCACATAGAACAGCACTCGACACAGAGTGAGCACTTGGCAACTGTTAGCTGTTACTAACCTTTCCC
ATTCTTCCTCCAAACCTATTCCAACTATCTGAATCATGTGCCCCTTCTCTGTGAACCTCTATCATAATA
CTTGTCACACTGTATTGTAATTGTCTCTTTTACTTTCCCTTGTATCTTTTGTGCATAGCAGAGTACCTG
AAACAGGAAGTATTTTAAATATTTTGAATCAAATGAGTTAATAGAATCTTTACAAATAAGAATATACAC
TTCTGCTTAGGATGATAATTGGAGGCAAGTGAATCCTGAGCGTGATTTGATAATGACCTAATAATGATG
GGTTTTATTTCCAG

TABLE 1
Indel screening gRNAs with their corresponding
Indel and Cell Survival Rates.

| | | | | Cell | | |
|---|---|---|---|---|---|---|
| | | INDEL | | Survival | | SEQ |
| | | Rates | SD | Rates | SD | ID |
| gRNA ID | gRNA spacer sequences | (%) | (%) | (%) | (%) | NO: |
| No SgRNA | N/A - Negative Control | 2.0 | 1.3 | 100.0 | 0.4 | |
| VEGFA | CCAUUCCCUCUUUAGCCAGAGC | 89.6 | 0.6 | 93.7 | 1.8 | 39 |
| 278 | UGGCGAGCAUUCAAUAACUUUA | 0.8 | 0.8 | 98.4 | 1.4 | 40 |
| 306 | GUUCUGGUUAAGAAUGGAUAAA | 2.4 | 1.7 | 96.7 | 1.2 | 41 |
| 315 | AAAAUGUCUGUUCUGGUUAAGA | 5.2 | 3.6 | 95.5 | 0.9 | 42 |
| 320 | UGAAAAAAUGUCUGUUCUGGUU | 4.3 | 0.6 | 89.0 | 3.0 | 43 |
| 505 | AUAGUUUUUUGAGGAAAUACAU | 4.8 | 1.9 | 88.5 | 2.5 | 44 |
| 550 | CCAGGUUAAGUUGUUCUUAGGG | 1.8 | 0.3 | 96.5 | 2.5 | 45 |
| 555 | AUAUGCCAGGUUAAGUUGUUCU | 1.9 | 0.7 | 94.4 | 2.2 | 46 |
| 664 | AGAAGACUUUGUAGUGAUCUUC | 3.3 | 0.6 | 93.8 | 1.7 | 47 |
| 781 | AUGAGAAAGAGCUUUCUAGUAU | 2.7 | 0.6 | 93.6 | 3.1 | 48 |
| 1119 | UAUUGAUAAAUAAUUGCCUUUC | 3.0 | 0.3 | 96.1 | 3.3 | 49 |
| 1159 | AGAUGAGGAGAACCAUAUUUUG | 3.5 | 1.0 | 94.4 | 1.6 | 50 |
| 1220** | ACCCAGCCUGACACCAAAUUUA | 33.4 | 4.7 | 92.6 | 2.1 | 51 |
| 1229 | ACUCAAUAAAUUUGGUGUCAGG | 7.0 | 0.9 | 90.8 | 3.1 | 52 |
| 1760 | AUAAGUUAGCAAUGGUCUAAAC | 4.4 | 1.9 | 86.0 | 3.8 | 53 |
| 1799 | AAAAGGUAGUAAUUGUGUUUCA | 2.9 | 1.2 | 87.5 | 1.9 | 54 |
| 1829 | GAAUUGAUCUUGAAGACAUACG | 3.5 | 0.3 | 97.9 | 1.3 | 55 |
| 1853* | AGCUAUUUUUGCACAUCUAUCA | 8.9 | 0.8 | 96.6 | 0.4 | 56 |
| 1869 | GAUAGAUGUGCAAAAAUAGCUU | 4.9 | 0.9 | 96.4 | 2.0 | 57 |
| 1893 | AAAUUUAAUUGCCAGUAAGUCU | 5.3 | 0.8 | 95.3 | 2.0 | 58 |
| 1989 | UUAUGAAAAGCAGCUAUGAAGG | 5.7 | 1.0 | 92.6 | 1.7 | 59 |
| 2017 | UAGAGGAAGGCAGUGGUCCCUU | 3.7 | 0.4 | 92.0 | 2.0 | 60 |
| 2068** | UACUAAAAGGCAGCCUCCUAGA | 35.0 | 4.1 | 93.4 | 3.5 | 61 |
| 2088 | GAGGCUGCCUUUUAGUAGUAUU | 6.4 | 0.4 | 97.5 | 2.7 | 62 |
| 2144 | AAGAUCUAGCUAAAAUAUAAGA | 3.8 | 0.3 | 100.6 | 1.0 | 63 |
| 2197* | ACAAAUUACCAAAUUGUAUUGA | 36.3 | 4.9 | 93.2 | 1.7 | 64 |
| 2272 | CAUAAGAUCUCUGCAAACAUAU | 5.3 | 0.3 | 96.1 | 1.1 | 65 |
| 2327 | CACUGCUUUCAGGAGCCAAAAA | 3.2 | 0.3 | 97.9 | 2.2 | 66 |
| 2351 | GGGUGGGGGAGCCCCAUAAAUG | 5.9 | 1.0 | 95.9 | 3.0 | 67 |
| 2360* | AGCCCCAUAAAUGUUGAAUAAU | 18.8 | 2.9 | 94.4 | 2.5 | 68 |
| 2431* | UCAGCCAAUAAAAGGUUUGUUA | 31.6 | 2.9 | 94.4 | 1.4 | 69 |
| 2451 | GGUUUGUUAAAGAAUGACUGUG | 5.8 | 1.8 | 91.3 | 3.3 | 70 |
| 2476* | AUGAUAUACUCUUAAGUGAAUA | 36.1 | 5.8 | 94.3 | 1.5 | 71 |
| 2697* | GCCUAGAUGAUUAUUAAUAGGG | 16.6 | 2.9 | 89.3 | 1.8 | 72 |
| 2698 | GCCUAGAUGAUUAUUAAUAGGG | 7.2 | 0.2 | 88.1 | 1.3 | 73 |
| 2717 | UUAAUAAUCAUCUAGGCUGCAA | 3.8 | 0.2 | 90.1 | 3.6 | 74 |
| 2756 | CUAGGCACUUGUUGACAGUCCU | 4.9 | 0.5 | 94.3 | 1.4 | 75 |
| 2878* | GUCUUGCGCUUAUGAAACUUCC | 12.5 | 2.0 | 94.1 | 3.0 | 76 |
| 2908* | UUUUAGUGAAGCAAUAUUAGUA | 28.6 | 4.6 | 94.4 | 5.1 | 77 |
| 2923 | AUUAGUAUAGAAUUUUGCAUCA | 2.3 | 0.6 | 95.6 | 1.2 | 78 |
| 2972* | UAUGGCAUCUCACCAGUGUGUG | 29.0 | 6.1 | 86.9 | 2.8 | 79 |
| 3042* | AUAAUAGAGACAAGGUGGUGUC | 14.9 | 3.1 | 88.8 | 1.9 | 80 |
| 3079* | UGAUCAUUGCCUCACUAUGGUA | 24.0 | 6.0 | 87.4 | 1.1 | 81 |
| 3106 | ACAUAUGAGUCCCAGUUAUUAC | 5.7 | 1.0 | 92.0 | 2.4 | 82 |
| 3122 | UAAUAACUGGGACUCAUAUGUA | 4.5 | 0.6 | 90.8 | 4.4 | 83 |
| 3125* | GCUAAUAGCUUAUUCUAUACAU | 38.5 | 2.9 | 91.9 | 3.1 | 84 |
| 3614 | UUCUUAUUACAUGGGAAAAAAU | 2.0 | 0.7 | 95.0 | 2.1 | 85 |
| 3630 | UUCAGAUUUUUUCCCAUGUAAU | 3.1 | 1.2 | 95.1 | 2.8 | 86 |
| 3687 | UGUUACAUGACCUUCCUUUCUU | 1.5 | 0.8 | 91.6 | 4.2 | 87 |
| 3821** | AUUGGCUACCUUGGUUGGAUGA | 54.8 | 3.6 | 80.1 | 4.1 | 88 |
| 3829* | UUACCUGGAUUGGCUACCUUGG | 7.6 | 0.6 | 95.2 | 3.2 | 89 |
| 3922 | UUGUUACAUAAAAAGAGAGGUG | 1.8 | 0.1 | 97.3 | 0.9 | 90 |
| 4000 | AUAUGUGCAAGUCCUCUGUGCU | 2.8 | 0.6 | 97.2 | 1.9 | 91 |
| 4025 | ACUUGCACAUAUGUUCAGACUG | 7.3 | 0.6 | 95.3 | 3.7 | 92 |
| 4195* | GGUGAGGGUCUCUCUAAGGUGU | 23.8 | 3.3 | 96.1 | 4.5 | 93 |
| 4215* | UUCUCACAUGGCAGAAAGGGGU | 34.6 | 2.4 | 86.1 | 2.1 | 94 |
| 4254 | AGCAGGAAGACAGCUGGCUAUC | 5.9 | 0.9 | 94.4 | 4.5 | 95 |
| 4262** | GACAGCUGGCUAUCCAGGAUUC | 82.4 | 1.9 | 90.8 | 5.1 | 96 |
| 4264 | UCUGCUAAGAGACUCCUGAAUC | 1.6 | 0.6 | 101.0 | 2.7 | 97 |
| 4271* | AUUUGGGUCUGCUAAGAGACUC | 42.0 | 1.4 | 97.0 | 2.7 | 98 |
| 4463 | CAUAGAAUAUCCAUCCAUAUGA | 6.6 | 0.8 | 102.2 | 2.7 | 99 |
| 4470 | UGCAUAGUAUUCUAUCAUAUGG | 4.1 | 0.2 | 101.6 | 1.4 | 100 |
| 4482 | UGGAUGGAUAUUCUAUGAUAUA | 3.0 | 0.6 | 99.4 | 1.6 | 101 |
| 4692* | AUUUAGAGUGUAUGGCAUGAGU | 28.2 | 3.6 | 92.6 | 3.4 | 102 |
| 4698* | CACUCUAUUUAGAGUGUAUGGC | 30.1 | 3.3 | 94.4 | 3.4 | 103 |
| 4706* | UACUCAUGCCAUACACUCUAAA | 29.9 | 1.7 | 91.5 | 2.4 | 104 |
| 4711* | GUGGGGAAAUCAGCACUCUAUU | 11.6 | 1.3 | 94.6 | 3.4 | 105 |
| 4861 | AUCAUUUCAACUUAUACACAGG | 3.8 | 1.1 | 93.2 | 3.7 | 106 |
| 4889* | UUCUCUCUAUAUAAAGUGAUCC | 15.3 | 2.0 | 96.3 | 2.2 | 107 |
| 4947 | CAUUAGGAAAAUGUACAAAGGA | 2.3 | 0.4 | 101.0 | 4.1 | 108 |
| 5041** | ACUUGCAGGAGGUGAGGGAUUA | 89.4 | 1.7 | 97.1 | 3.6 | 109 |
| 5052** | AUUAGGGAAUGCAGACUCUGGG | 45.9 | 1.9 | 88.6 | 1.7 | 110 |
| 5057* | CUGCUACUUGGAAGACUCUUCC | 46.6 | 3.8 | 95.4 | 1.6 | 111 |
| 5169* | GGCUGGCAUAGAGUAAGACAGG | 29.9 | 3.1 | 96.8 | 2.1 | 112 |
| 5211 | GAAGUAAUCGGCGGUGGAGGUA | 2.2 | 0.4 | 96.6 | 1.7 | 113 |
| 5278** | UGGGUGAGAUUAGAGGCCACUG | 43.4 | 2.7 | 90.6 | 3.0 | 114 |
| 5343** | UGCUUCCUCCCUUGUCUCCCUA | 52.2 | 5.4 | 92.4 | 2.5 | 115 |
| 5426* | ACUAUACUACUUCCACUACUAC | 40.8 | 2.9 | 95.7 | 1.9 | 116 |
| 5491 | UUCAACUUAUCCAUUAAUCCUA | 7.2 | 1.2 | 94.4 | 2.5 | 117 |
| 5492* | GAAGGAAGAUCCAAUAGGAUUA | 18.5 | 2.4 | 95.3 | 2.8 | 118 |
| 5538** | UGGCAUAUGAGAAAAGUCACAG | 72.4 | 1.0 | 92.4 | 1.6 | 119 |
| 5628 | AAGAUAAUGGGAGAAACAGGUU | 3.5 | 0.4 | 99.9 | 2.9 | 120 |
| 5683* | UACAUCCAACUAACAUCUCUAU | 18.6 | 2.1 | 94.4 | 1.9 | 121 |
| 5689 | GACAUCCAAAUAGAGAUGUUAG | 7.3 | 0.8 | 96.2 | 1.8 | 122 |
| 5744 | GGCUGGUAGUGUGAAGAUGGGG | 1.4 | 0.3 | 92.5 | 3.0 | 123 |
| 5766 | UUCUUAUCAACCCUCUUUCCUU | 1.2 | 0.1 | 97.5 | 3.2 | 124 |
| 5793 | AAGAGGGUUGAUAAGAAGAGAA | 1.5 | 0.1 | 95.1 | 2.5 | 125 |
| 5801* | UGAUAAGAAGAGAAAGGGGUGU | 13.8 | 2.6 | 102.9 | 5.1 | 126 |
| 5825 | CACCUCCUUAACCUCUAAUACU | 2.6 | 0.2 | 94.3 | 2.7 | 127 |
| 5837* | CUAAGUAUUAGAGGUUAAGGAG | 19.8 | 2.6 | 92.0 | 0.5 | 128 |
| 5896* | UCUGAGAAGACAAAGCUAGUGA | 25.9 | 1.1 | 99.9 | 3.6 | 129 |
| 5913 | CUGCUUGACAUCAGUUGGGUUC | 5.5 | 0.9 | 98.7 | 1.9 | 130 |
| 5921 | ACACCCUUCUGCUUGACAUCAG | 7.2 | 1.4 | 96.9 | 2.5 | 131 |
| 5928* | GAACCCAACUGAUGUCAAGCAG | 18.1 | 2.8 | 91.4 | 2.0 | 132 |
| 6029* | CUACUAUUGCUUUCAUUAAGUC | 13.0 | 0.6 | 91.6 | 3.3 | 133 |
| 6061 | AGUAGAGAAGGUCAAACUUGAC | 1.3 | 0.5 | 94.8 | 3.6 | 134 |
| 6083 | ACUUGACCAGAAUGAAAUUAGA | 2.9 | 0.2 | 99.8 | 3.0 | 135 |
| 6138* | GUGGACUCCAGGAGUAUAUCAA | 8.6 | 0.7 | 98.8 | 2.3 | 136 |
| 6150** | CCUUAUUCUUUUGAUAUACUCC | 44.8 | 5.3 | 93.3 | 2.4 | 137 |
| 6151* | AUUGUAUUAGCAAGUGGACUCC | 22.1 | 0.9 | 89.9 | 1.5 | 138 |
| 6286* | GUAUUUCUAGGCUAGACAGUUU | 47.2 | 2.2 | 93.8 | 1.8 | 139 |
| 6460* | CUUUACUGUGAAUGGUGCCAGG | 9.0 | 2.1 | 95.8 | 2.1 | 140 |
| 6603 | UAUUACGGUAAGCUCAAGCAUU | 1.9 | 0.4 | 97.3 | 1.5 | 141 |
| 7055* | CUUAACUACUAAUAGCCUAUUG | 16.4 | 2.1 | 84.1 | 3.3 | 142 |
| 7152* | ACCCCUAACCUUCAUGUUGAUA | 16.2 | 1.2 | 94.6 | 2.1 | 143 |
| 7625 | AAUAGCACUGGAGUUACCUGUU | 5.1 | 0.3 | 94.1 | 3.6 | 144 |
| 7652 | UUUUUUUUCCAAGCGGUCCCUU | 6.1 | 0.6 | 95.2 | 3.7 | 145 |
| 8379 | UGGAAAAUGUUAAAUUUUCCCU | 0.9 | 0.2 | 99.3 | 4.1 | 146 |
| 8519 | UACUUGCUCUCUGACCACAUAA | 3.0 | 0.3 | 98.2 | 2.3 | 147 |
| 8541 | CUAAUCUAUUAAUAGAAACUAC | 5.3 | 0.6 | 92.6 | 4.3 | 148 |
| 8600 | AGAUGUAGAAGACCUAAAUAUC | 2.8 | 0.3 | 97.2 | 4.1 | 149 |
| 8784 | AAGAAAAGGCUCUUUAUAAUGC | 4.7 | 0.2 | 98.1 | 1.9 | 150 |
| 8922* | UAAAAGAGACACACCUAAAGCA | 27.6 | 2.5 | 90.0 | 3.8 | 151 |
| 9000 | UGAAGUCUUUUCCUCUUAAUAG | 2.8 | 0.6 | 94.1 | 2.5 | 152 |
| 9468 | AUAUUAGUGAACUAGAGUCACA | 1.5 | 0.2 | 98.4 | 2.2 | 153 |
| 9478* | AUCAGAGCUCAUAUUAGUGAAC | 28.8 | 4.6 | 93.7 | 1.4 | 154 |
| 10303 | AGCACAUAGAACAGCACUCGAC | 0.0 | 0.0 | 99.7 | 1.2 | 155 |
| 10308* | GUUGCCAAGUGCUCACUCUGUG | 21.7 | 5.3 | 84.9 | 2.7 | 156 |
| 10375* | CCUCCAAACCUAUUCCAACUAU | 18.1 | 5.5 | 92.8 | 1.0 | 157 |
| 10377 | AGGGGCACAUGAUUCAGAUAGU | 1.0 | 0.7 | 95.6 | 1.9 | 158 |

Note:
Indel and cell survival rates represent the mean of 2 independent experiments where each sgRNA was run in triplicate. Each sgRNA contain a unique 22 nucleotides spacer sequence follow by a common 80 nucleotides scaffold sequence. gRNA scaffold sequence (5′-3′): GUUUUAGUACUCUGGAAACAGAAUCUACUAAAACAAGGCAAAAUGCCGUGUUUAUCUCGUCAACUUGUUGGCGAGAUUUU (SEQ ID NO: 12)
*gRNAs with indel rates above 7.5% threshold were considered as positive hits. Threshold value was determined by adding 4 SD to negative control mean.
**10 candidate gRNAs were selected for further analysis based on location, indel percentage, and cell survival rates.

CFTR Super-Exon AAV Donor Construct Design and AAV Transduction CFTR intron 10 AAV donor construct contained: (i) 500 nucleotide long left and right homology arms relative to gRNA cut site, (ii) a splice acceptor site, (iii) cDNA of wild type CFTR gene since exon 11 until 27, and (iv) a stop and polyadenylation sequence. AAV donor preparations were made by using a AAV6 serotype, purified and titrated by Vector Biolabs. AAV titration was reported as viral genomes per mL. AAV transductions were performed by adding AAV6 vector into cells at specified vector genome copies per cell for 36 hours at 37° C.

Electroporation: Electroporations were performed by using the Lonza 4D-Nucleofector™ System coupled to 96-well shuttle system. 1.8×10⁵ LPC cells were resuspended in 20 μL of P4 Electroporation buffer Lonza (V4SP-4096). 20 μL of cell mixture was combined with 2 μL of CRISPR-Cas9 reagent mix containing 1 μg of saCAS9 mRNA and 1 μg of gRNA. 20 μL of cell and CRISPR-Cas9 mixture was transferred into one well of a 96-well electroporation plate. Cells were electroporated by using program CM-138. A fraction of electroporated LPC cells were transferred into a well of a 384-well plate or an ALI-96-well plate containing LPCs culture media. Cells were incubated at 37° C. in a 5% CO2 incubator.

Genomic DNA Isolation: Genomic DNA was isolated by incubating cells for 30 min at 37° C. with 50 μL and 15 μL of DNA Quick extract solution (Epicentre) per well of a 96-well and a 384-well plate, respectively. Cellular extract was mixed and transferred into a 96-well PCR plate and then incubated for 6 min at 65° C. and 2 min at 98° C. Genomic DNA was immediately use in downstream applications or it was store at 4° C.

Measurement of INDEL Rates for CFTR Intron gRNA Screening: Long-range PCR PrimeSTAR kit (R045B, TAKARA) was used to amplify a 12 Kb DNA fragment corresponding to CFTR intron 10. PCR reactions were carried out following manufacturer instructions. In brief, 2.5 μL of genomic DNA solution was combined with 47.5 μL of PrimeSTAR master mix containing dNTPS, 5×GXL buffer, GXL Polymerase and the corresponding CFTR Intron 10 forward and reverse primers (CFTR 12 Kb F3: GCTACCAGTGTGATGGAGTAG (SEQ ID NO: 160) and CFTR 12 Kb R3: AGCCAGGGATACAATATCTTCACAA (SEQ ID NO: 161) at 250 nM each). PCR reactions were performed using the following thermal cycling protocol: 1) 94° C. 30 s; 2), 94° C. 10 s; 3), 68° C. 10 min; 4) repeat steps 2-3 32 times.

PCR reactions for the VEGFA positive control were performed using Phire Green Hot Start II PCR Master Mix kit (F126L, Thermo Scientific) following manufacturer instructions. In brief, 1.5 μL of genomic DNA solution was combined with 18.5 μL of Phire master mix with the corresponding VEGFA forward and reverse primer pair (VEGFA-F1: CCAGTCACTGACTAACCCCG (SEQ ID NO: 162) and VEGFA-R1: ACTCTGTCCAGAGACACGCG (SEQ ID NO: 163) at 625 nM each). PCR reactions were performed using the following thermal cycling protocol: 1) 98° C. 30 s; 2) 98° C. 5 s; 3) 60° C. 5 s; 472°) C. 10 s 5) repeat steps 2-4 35 times; 6), 72° C. 4 min.

The PCR products were enzymatically purified, and CFTR intron 10 PCR products were amplified using Rolling Cycle Amplification at GENEWIZ. DNA samples were Sanger sequenced using sequencing primers that bind near the cut site of sgRNA tested (TABLE 2). Each sequencing chromatogram was analyzed using TIDE software against reference sequences (tide.nki.nl). References sequences were obtained from mock-electroporated samples. Tide parameters were set to cover an indel spectrum of +/−30 nucleotides of gRNA cut site and the decomposition window was set to cover the largest possible window with high-quality traces. Total indel (insertion and deletions) rates were obtained directly from TIDE plots. GraphPad Prism 7 software was used to make Graphs and to calculate the all Statistical information.

TABLE 2
Sequencing primers for CFTR indel gRNA
screening and VEGFA reference control.

| gRNA | Primer | | Sequencing | SEQ ID |
|---|---|---|---|---|
| ID | name | Sequence | protocol | NO: |
| VEGFA | VEGFA-F1 | CCAGTCACTGACTAACCCCG | Standard | 164 |
| | | | sequencing | |
| 278 | CFTR-F1 | AACTTCTAATGGTGATGACA | RCA + Standard | 165 |
| | | GCC | sequencing | |
| 306 | CFTR-R1 | GTCATGATTTTCCTGGACCA | RCA + Standard | 166 |
| | | GC | sequencing | |
| 315 | CFTR-R1 | GTCATGATTTTCCTGGACCA | RCA + Standard | 167 |
| | | GC | sequencing | |
| 320 | CFTR-R1 | GTCATGATTTTCCTGGACCA | RCA + Standard | 168 |
| | | GC | sequencing | |
| 505 | CFTR-R1 | GTCATGATTTTCCTGGACCA | RCA + Standard | 169 |
| | | GC | sequencing | |
| 550 | CFTR-R1 | GTCATGATTTTCCTGGACCA | RCA + Standard | 170 |
| | | GC | sequencing | |
| 555 | CFTR-R1 | GTCATGATTTTCCTGGACCA | RCA + Standard | 171 |
| | | GC | sequencing | |
| 664 | CFTR-R2 | GGGTACAGTGGTGCAATCAT | RCA + Standard | 172 |
| | | GG | sequencing | |
| 781 | CFTR-R2 | GGGTACAGTGGTGCAATCAT | RCA + Standard | 173 |
| | | GG | sequencing | |
| 1119 | CFTR-R2 | GGGTACAGTGGTGCAATCAT | RCA + Standard | 174 |
| | | GG | sequencing | |
| 1159 | CFTR-R2 | GGGTACAGTGGTGCAATCAT | RCA + Standard | 175 |
| | | GG | sequencing | |
| 1220** | CFTR-R2 | GGGTACAGTGGTGCAATCAT | RCA + Standard | 176 |
| | | GG | sequencing | |
| 1229 | CFTR-R2 | GGGTACAGTGGTGCAATCAT | RCA + Standard | 177 |
| | | GG | sequencing | |
| 1760 | CFTR-R3 | AAAAGGCAGCCTCCTAGATT | RCA + Standard | 178 |
| | | GA | sequencing | |
| 1799 | CFTR-R3 | AAAAGGCAGCCTCCTAGATT | RCA + Standard | 179 |
| | | GA | sequencing | |
| 1829 | CFTR-R3 | AAAAGGCAGCCTCCTAGATT | RCA + Standard | 180 |
| | | GA | sequencing | |
| 1853* | CFTR-R3 | AAAAGGCAGCCTCCTAGATT | RCA + Standard | 181 |
| | | GA | sequencing | |
| 1869 | CFTR-R3 | AAAAGGCAGCCTCCTAGATT | RCA + Standard | 182 |
| | | GA | sequencing | |
| 1893 | CFTR-R3 | AAAAGGCAGCCTCCTAGATT | RCA + Standard | 183 |
| | | GA | sequencing | |
| 1989 | CFTR-F4 | TGCGTATGTCTTCAAGATCA | RCA + Standard | 184 |
| | | ATTCT | sequencing | |
| 2017 | CFTR-F4 | TGCGTATGTCTTCAAGATCA | RCA + Standard | 185 |
| | | ATTCT | sequencing | |
| 2068** | CFTR-F4 | TGCGTATGTCTTCAAGATCA | RCA + Standard | 186 |
| | | ATTCT | sequencing | |
| 2088 | CFTR-F4 | TGCGTATGTCTTCAAGATCA | RCA + Standard | 187 |
| | | ATTCT | sequencing | |
| 2144 | CFTR-F4 | TGCGTATGTCTTCAAGATCA | RCA + Standard | 188 |
| | | ATTCT | sequencing | |
| 2197* | CFTR-F4 | TGCGTATGTCTTCAAGATCA | RCA + Standard | 189 |
| | | ATTCT | sequencing | |
| 2272 | CFTR-F4 | TGCGTATGTCTTCAAGATCA | RCA + Standard | 190 |
| | | ATTCT | sequencing | |
| 2327 | CFTR-F5 | TTGGACAAAAGATCTAGCTA | RCA + Standard | 191 |
| | | AAATATAAGATTGA | sequencing | |
| 2351 | CFTR-F5 | TTGGACAAAAGATCTAGCTA | RCA + Standard | 192 |
| | | AAATATAAGATTGA | sequencing | |
| 2360* | CFTR-F5 | TTGGACAAAAGATCTAGCTA | RCA + Standard | 193 |
| | | AAATATAAGATTGA | sequencing | |
| 2431* | CFTR-F5 | TTGGACAAAAGATCTAGCTA | RCA + Standard | 194 |
| | | AAATATAAGATTGA | sequencing | |
| 2451 | CFTR-F5 | TTGGACAAAAGATCTAGCTA | RCA + Standard | 195 |
| | | AAATATAAGATTGA | sequencing | |
| 2476* | CFTR-F5 | TTGGACAAAAGATCTAGCTA | RCA + Standard | 196 |
| | | AAATATAAGATTGA | sequencing | |
| 2697* | CFTR-R5 | GGCATCCAGTGATGCAAAAT | RCA + Standard | 197 |
| | | TCTATAC | sequencing | |
| 2698 | CFTR-R5 | GGCATCCAGTGATGCAAAAT | RCA + Standard | 198 |
| | | TCTATAC | sequencing | |
| 2717 | CFTR-R5 | GGCATCCAGTGATGCAAAAT | RCA + Standard | 199 |
| | | TCTATAC | sequencing | |
| 2756 | CFTR-R5 | GGCATCCAGTGATGCAAAAT | RCA + Standard | 200 |
| | | TCTATAC | sequencing | |
| 2878* | CFTR-F6 | AGTTGTTATCTCTGAAATTT | RCA + Standard | 201 |
| | | TGGGT | sequencing | |
| 2908* | CFTR-F6 | AGTTGTTATCTCTGAAATTT | RCA + Standard | 202 |
| | | TGGGT | sequencing | |
| 2923 | CFTR-F6 | AGTTGTTATCTCTGAAATTT | RCA + Standard | 203 |
| | | TGGGT | sequencing | |
| 2972* | CFTR-F6 | AGTTGTTATCTCTGAAATTT | RCA + Standard | 204 |
| | | TGGGT | sequencing | |
| 3042* | CFTR-R6 | ACAATATAGCTCTGATAATC | RCA + Standard | 205 |
| | | CAGGT | sequencing | |
| 3079* | CFTR-R6 | ACAATATAGCTCTGATAATC | RCA + Standard | 206 |
| | | CAGGT | sequencing | |
| 3106 | CFTR-R6 | ACAATATAGCTCTGATAATC | RCA + Standard | 207 |
| | | CAGGT | sequencing | |
| 3122 | CFTR-R6 | ACAATATAGCTCTGATAATC | RCA + Standard | 208 |
| | | CAGGT | sequencing | |
| 3125* | CFTR-R6 | ACAATATAGCTCTGATAATC | RCA + Standard | 209 |
| | | CAGGT | sequencing | |
| 3614 | CFTR-R6B | CCCCACCCTGCAATACCA | RCA + Standard | 210 |
| | | | sequencing | |
| 3630 | CFTR-R6B | CCCCACCCTGCAATACCA | RCA + Standard | 211 |
| | | | sequencing | |
| 3687 | CFTR-R6B | CCCCACCCTGCAATACCA | RCA + Standard | 212 |
| | | | sequencing | |
| 3821** | CFTR-R6B | CCCCACCCTGCAATACCA | RCA + Standard | 213 |
| | | | sequencing | |
| 3829* | CFTR-R6B | CCCCACCCTGCAATACCA | RCA + Standard | 214 |
| | | | sequencing | |
| 3922 | CFTR-F7 | CCTGCATTCCCTCATCCA | RCA − GC rich | 215 |
| | | | protocol | |
| 4000 | CFTR-F7 | CCTGCATTCCCTCATCCA | RCA − GC rich | 216 |
| | | | protocol | |
| 4025 | CFTR-F7 | CCTGCATTCCCTCATCCA | RCA − GC rich | 217 |
| | | | protocol | |
| 4195* | CFTR-F7 | CCTGCATTCCCTCATCCA | RCA − GC rich | 218 |
| | | | protocol | |
| 4215* | CFTR-F7 | CCTGCATTCCCTCATCCA | RCA − GC rich | 219 |
| | | | protocol | |
| 4254 | CFTR-F7 | CCTGCATTCCCTCATCCA | RCA − GC rich | 220 |
| | | | protocol | |
| 4262** | CFTR-F7 | CCTGCATTCCCTCATCCA | RCA − GC rich | 221 |
| | | | protocol | |
| 4264 | CFTR-F7 | CCTGCATTCCCTCATCCA | RCA − GC rich | 222 |
| | | | protocol | |
| 4271* | CFTR-F7 | CCTGCATTCCCTCATCCA | RCA − GC rich | 223 |
| | | | protocol | |
| 4463 | CFTR-R7 | GCTGTGGGGAAATCAGCA | RCA + Standard | 224 |
| | | | sequencing | |
| 4470 | CFTR-R7 | GCTGTGGGGAAATCAGCA | RCA + Standard | 225 |
| | | | sequencing | |
| 4482 | CFTR-R7 | GCTGTGGGGAAATCAGCA | RCA + Standard | 226 |
| | | | sequencing | |
| 4692* | CFTR-F8 | TGCACACACATTTAAATAGA | RCA + Standard | 227 |
| | | TGCATAGT | sequencing | |
| 4698* | CFTR-F8 | TGCACACACATTTAAATAGA | RCA + Standard | 228 |
| | | TGCATAGT | sequencing | |
| 4706* | CFTR-F8 | TGCACACACATTTAAATAGA | RCA + Standard | 229 |
| | | TGCATAGT | sequencing | |
| 4711* | CFTR-F8 | TGCACACACATTTAAATAGA | RCA + Standard | 230 |
| | | TGCATAGT | sequencing | |
| 4861 | CFTR-F8 | TGCACACACATTTAAATAGA | RCA + Standard | 231 |
| | | TGCATAGT | sequencing | |
| 4889* | CFTR-F8 | TGCACACACATTTAAATAGA | RCA + Standard | 232 |
| | | TGCATAGT | sequencing | |
| 4947 | CFTR-R8 | TTGCCTGAACTGGCACAAT | RCA + Standard | 233 |
| | | | sequencing | |
| 5041** | CFTR-R8 | TTGCCTGAACTGGCACAAT | RCA + Standard | 234 |
| | | | sequencing | |
| 5052** | CFTR-R8 | TTGCCTGAACTGGCACAAT | RCA + Standard | 235 |
| | | | sequencing | |
| 5057* | CFTR-R8 | TTGCCTGAACTGGCACAAT | RCA + Standard | 236 |
| | | | sequencing | |
| 5169* | CFTR-F9 | TTGCAGGAGGTGAGGGATT | RCA + Standard | 237 |
| | | | sequencing | |
| 5211 | CFTR-F9 | TTGCAGGAGGTGAGGGATT | RCA + Standard | 238 |
| | | | sequencing | |
| 5278** | CFTR-F9 | TTGCAGGAGGTGAGGGATT | RCA + Standard | 239 |
| | | | sequencing | |
| 5343** | CFTR-F9 | TTGCAGGAGGTGAGGGATT | RCA + Standard | 240 |
| | | | sequencing | |
| 5426* | CFTR-F9 | TTGCAGGAGGTGAGGGATT | RCA + Standard | 241 |
| | | | sequencing | |
| 5491 | CFTR-F9 | TTGCAGGAGGTGAGGGATT | RCA + Standard | 242 |
| | | | sequencing | |
| 5492* | CFTR-F9 | TTGCAGGAGGTGAGGGATT | RCA + Standard | 243 |
| | | | sequencing | |
| 5538** | CFTR-R9 | TCAGTTGGGTTCCGGGATA | RCA + Standard | 244 |
| | | | sequencing | |
| 5628 | CFTR-R9 | TCAGTTGGGTTCCGGGATA | RCA + Standard | 245 |
| | | | sequencing | |
| 5683* | CFTR-R9 | TCAGTTGGGTTCCGGGATA | RCA + Standard | 246 |
| | | | sequencing | |
| 5689 | CFTR-R9 | TCAGTTGGGTTCCGGGATA | RCA + Standard | 247 |
| | | | sequencing | |
| 5744 | CFTR-F10 | GTCACAGAGGAGTCAAAGAT | RCA + Standard | 248 |
| | | GATTCCAAG | sequencing | |
| 5766 | CFTR-F10 | GTCACAGAGGAGTCAAAGAT | RCA + Standard | 249 |
| | | GATTCCAAG | sequencing | |
| 5793 | CFTR-F10 | GTCACAGAGGAGTCAAAGAT | RCA + Standard | 250 |
| | | GATTCCAAG | sequencing | |
| 5801* | CFTR-F10 | GTCACAGAGGAGTCAAAGAT | RCA + Standard | 251 |
| | | GATTCCAAG | sequencing | |
| 5825 | CFTR-F10 | GTCACAGAGGAGTCAAAGAT | RCA + Standard | 252 |
| | | GATTCCAAG | sequencing | |
| 5837* | CFTR-F10 | GTCACAGAGGAGTCAAAGAT | RCA + Standard | 253 |
| | | GATTCCAAG | sequencing | |
| 5896* | CFTR-F10 | GTCACAGAGGAGTCAAAGAT | RCA + Standard | 254 |
| | | GATTCCAAG | sequencing | |
| 5913 | CFTR-F10 | GTCACAGAGGAGTCAAAGAT | RCA + Standard | 255 |
| | | GATTCCAAG | sequencing | |
| 5921 | CFTR-F10 | GTCACAGAGGAGTCAAAGAT | RCA + Standard | 256 |
| | | GATTCCAAG | sequencing | |
| 5928* | CFTR-F10 | GTCACAGAGGAGTCAAAGAT | RCA + Standard | 257 |
| | | GATTCCAAG | sequencing | |
| 6029* | CFTR-R10 | GGGTAGGTGTTAGTGTGTGT | RCA + Standard | 258 |
| | | TAAGT | sequencing | |
| 6061 | CFTR-R10 | GGGTAGGTGTTAGTGTGTGT | RCA + Standard | 259 |
| | | TAAGT | sequencing | |
| 6083 | CFTR-R10 | GGGTAGGTGTTAGTGTGTGT | RCA + Standard | 260 |
| | | TAAGT | sequencing | |
| 6138* | CFTR-R10 | GGGTAGGTGTTAGTGTGTGT | RCA + Standard | 261 |
| | | TAAGT | sequencing | |
| 6150** | CFTR-R10 | GGGTAGGTGTTAGTGTGTGT | RCA + Standard | 262 |
| | | TAAGT | sequencing | |
| 6151* | CFTR-R10 | GGGTAGGTGTTAGTGTGTGT | RCA + Standard | 263 |
| | | TAAGT | sequencing | |
| 6286* | CFTR-R10 | GGGTAGGTGTTAGTGTGTGT | RCA + Standard | 264 |
| | | TAAGT | sequencing | |
| 6460* | CFTR-F11 | TACAGGCTTGAACTGCATGG | RCA + Standard | 265 |
| | | | sequencing | |
| 6603 | CFTR-F11 | TACAGGCTTGAACTGCATGG | RCA + Standard | 266 |
| | | | sequencing | |
| 7055* | CFTR-F12 | CCACTAGTGATGCACGGAAA | RCA + Standard | 267 |
| | | | sequencing | |
| 7152* | CFTR-F12 | CCACTAGTGATGCACGGAAA | RCA + Standard | 268 |
| | | | sequencing | |
| 7625 | CFTR-F20 | CTCTGCCTCCCGGGCTCAAG | RCA + Standard | 269 |
| | | | sequencing | |
| 7652 | CFTR-F20 | CTCTGCCTCCCGGGCTCAAG | RCA + Standard | 270 |
| | | | sequencing | |
| 8379 | CFTR-F13 | AGCTGTGCATTTTCCTCTGG | RCA + Standard | 271 |
| | | | sequencing | |
| 8519 | CFTR-F13 | AGCTGTGCATTTTCCTCTGG | RCA + Standard | 272 |
| | | | sequencing | |
| 8541 | CFTR-F13 | AGCTGTGCATTTTCCTCTGG | RCA + Standard | 273 |
| | | | sequencing | |
| 8600 | CFTR-F13 | AGCTGTGCATTTTCCTCTGG | RCA + Standard | 274 |
| | | | sequencing | |
| 8784 | CFTR-R13 | TCTGTTAATGGCAAAGCAAG | RCA + Standard | 275 |
| | | TAGAA | sequencing | |
| 8922* | CFTR-R13 | TCTGTTAATGGCAAAGCAAG | RCA + Standard | 276 |
| | | TAGAA | sequencing | |
| 9000 | CFTR-R13 | TCTGTTAATGGCAAAGCAAG | RCA + Standard | 277 |
| | | TAGAA | sequencing | |
| 9468 | CFTR-F14 | GATGGGTTAAGCCAACTGAA | RCA + Standard | 278 |
| | | AA | sequencing | |
| 9478* | CFTR-F14 | GATGGGTTAAGCCAACTGAA | RCA + Standard | 279 |
| | | AA | sequencing | |
| 10303 | CFTR-R15 | ATTCACTTGCCTCCAATTAT | RCA + Standard | 280 |
| | | CATCCT | sequencing | |
| 10308* | CFTR-R15 | ATTCACTTGCCTCCAATTAT | RCA + Standard | 281 |
| | | CATCCT | sequencing | |
| 10375* | CFTR-R15 | ATTCACTTGCCTCCAATTAT | RCA + Standard | 282 |
| | | CATCCT | sequencing | |
| 10377 | CFTR-R15 | ATTCACTTGCCTCCAATTAT | RCA + Standard | 283 |
| | | CATCCT | sequencing | |

Measurement of CFTR Super-Exon Insertion Rates by Droplet Digital PCR (ddPCR): HDR-mediated insertion of CFTR super-exon 10 was assessed by ddPCR (QX200, Bio-Rad Laboratories, Inc.). Multiplex ddPCR assays were performed to specifically determine the amount of HDR-edited alleles relative to total alleles present in the sample. GAPDH allele count was used to determine the total number of alleles amplified by ddPCR. ddPCR were performed (1× assay: 900 nM primers, and 250 nM each probe) by using 2 ddPCR probes per assay (IDT, Inc.). One FAM-labeled probe was specific for HDR-edited alleles with one primer positioned outside of the template matching region of CFTR super-exon to prevent amplification of donor template. The second HEX-labeled probe and pair of primers were specific for GAPDH. ddPCR assays were assembled and droplets were generated by an Automated Droplet Generator (Bio-Rad). ddPCR assays were performed by using ddPCR supermix for probes (no dUTP) using the following thermal cycling protocol: 1) 95° C. 5 min; 2), 94° C. 30 s; 3), 58° C. 1 min; 4), 72° C. 3 min; 5) repeat steps 2-4 39 times; 6), 98° C. 5 min, with all the steps ramped by 2° C./s. QuantaSoft was used for quantification (Bio-Rad). The fraction of positive droplets corresponding to FAM and HEX fluorescent channels determines the amount of HDR-edited and GAPDH alleles, respectively. Percentage of HDR-edited alleles was calculated relative to GAPDH alleles which represent 100% of amplified alleles in the sample. The confidence intervals for each well were calculated by QuantaSoft based on Poisson distribution. Primers and probes sequences are shown in TABLE 3 and TABLE 4.

TABLE 3
Primers and Probes for CFTR Intron 10 ddPCR assays.

| | | | SEQ ID | | SEQ ID | ddPCR Probe | SEQ ID |
|---|---|---|---|---|---|---|---|
| gRNA | Side | Forward Primer | NO: | Reverse Primer | NO: | (FAM) | NO: |
| 1220 | 3′ | TACAGCGTAC | 284 | ACCACTGCCT | 285 | CATTGATGAGT | 286 |
| | | CTTCAGCTCA | | TCCTCTAACT | | TTGGACAAAC | |
| | | C | | C | | CACAACTAG | |
| | 5′ | CACATGGCGA | 287 | AGGCATGATC | 288 | AAGAAATCCG | 289 |
| | | GCATTCAATA | | CATGAGAACT | | TCCTGAGTGTT | |
| | | AC | | G | | TGATCTTCC | |
| 2068 | 3′ | TACAGCGTAC | 290 | GGAAGTTTCA | 291 | CATTGATGAGT | 292 |
| | | CTTCAGCTCA | | TAAGCGCAAG | | TTGGACAAAC | |
| | | C | | AC | | CACAACTAG | |
| | 5′ | AGAAAGACTC | 293 | AGGCATGATC | 294 | AAGAAATCCG | 295 |
| | | CTGAAAGGCA | | CATGAGAACT | | TCCTGAGTGTT | |
| | | | | G | | TGATCTTCC | |
| 3821 | 3′ | TACAGCGTAC | 296 | GAGTGTATGG | 297 | CATTGATGAGT | 298 |
| | | CTTCAGCTCA | | CATGAGTACG | | TTGGACAAAC | |
| | | C | | AAT | | CACAACTAG | |
| | 5′ | AATTCCTGAC | 299 | AGGCATGATC | 300 | AAGAAATCCG | 301 |
| | | ACCACCTTGT | | CATGAGAACT | | TCCTGAGTGTT | |
| | | CTC | | G | | TGATCTTCC | |
| 4262 | 3′ | TACAGCGTAC | 302 | ACCCTCCCTG | 303 | CATTGATGAGT | 304 |
| | | CTTCAGCTCA | | TCTTACTCTA | | TTGGACAAAC | |
| | | C | | TG | | CACAACTAG | |
| | 5′ | ATGCATATAT | 305 | AGGCATGATC | 306 | AAGAAATCCG | 307 |
| | | ATATTTTTAAC | | CATGAGAACT | | TCCTGAGTGTT | |
| | | CTGGATTATC | | G | | TGATCTTCC | |
| | | AGAGC | | | | | |
| 5041 | 3′ | TACAGCGTAC | 308 | CTTCACCCAC | 309 | CATTGATGAGT | 310 |
| 5052 | | CTTCAGCTCA | | CTCCTTAACC | | TTGGACAAAC | |
| | | C | | | | CACAACTAG | |
| | 5′ | GTGAGAACAC | 311 | AGGCATGATC | 312 | AAGAAATCCG | 313 |
| | | AGCAGGAAGA | | CATGAGAACT | | TCCTGAGTGTT | |
| | | C | | G | | TGATCTTCC | |
| 5278 | 3′ | TACAGCGTAC | 314 | AGGCATCAAT | 315 | CATTGATGAGT | 316 |
| 5343 | | CTTCAGCTCA | | GGTTGTCTGT | | TTGGACAAAC | |
| | | C | | ATT | | CACAACTAG | |
| | 5′ | ACTTCCCAGC | 317 | AGGCATGATC | 318 | AAGAAATCCG | 319 |
| | | CTCCAGAACT | | CATGAGAACT | | TCCTGAGTGTT | |
| | | | | G | | TGATCTTCC | |
| 5538 | 3′ | TACAGCGTAC | 320 | GTAGGGTAGG | 321 | CATTGATGAGT | 322 |
| | | CTTCAGCTCA | | TGTTAGTGTG | | TTGGACAAAC | |
| | | C | | TGTTAAG | | CACAACTAG | |
| | 5′ | GAGTGCTGAT | 323 | AGGCATGATC | 324 | AAGAAATCCG | 325 |
| | | TTCCCCACAG | | CATGAGAACT | | TCCTGAGTGTT | |
| | | | | G | | TGATCTTCC | |
| 6150 | 3′ | TACAGCGTAC | 326 | AGCTGCACTG | 327 | CATTGATGAGT | 328 |
| | | CTTCAGCTCA | | ATGGTTTCA | | TTGGACAAAC | |
| | | C | | | | CACAACTAG | |
| | 5′ | TAGGGAGACA | 329 | AGGCATGATC | 330 | AAGAAATCCG | 331 |
| | | AGGGAGGAAG | | CATGAGAACT | | TCCTGAGTGTT | |
| | | | | G | | TGATCTTCC | |

TABLE 4
Primers and Probes for GAPDH (Reference Assay to
Determine Total Number of Alleles).

| | Forward | SEQ ID | Reverse | SEQ ID | ddPCR | SEQ ID |
|---|---|---|---|---|---|---|
| | Primer | NO: | Primer | NO: | Probe (HEX) | NO: |
| GAPDH | TGGAAGACAGA | 332 | GTCAGGTCCAC | 333 | CCCCCACCCCCAT | 334 |
| | ATGGAAGAAAT | | CACTGA | | AGGCGAGATCCC | |

Measurement of LPC Cell Survival Rates: Cells were incubated with 5 μg/mL of Hoechst 33342 (Life technologies: H3570) and 0.5 μg/mL of Propidium Iodide (PI; Life technologies: P3566) in culture media for 1 hour at 37° C. Cells were imaged to measure Hoescht positive events (Live and death cells) and PI positive events (Death cells) by using a High-Content Imaging System (Molecular devices). Relative cell survival rate was calculated as follows: [(Hoescht+events −PI+events) of Sample]/(Hoescht+events −PI+events) of Control]*100. Control was Mock transfected cells and its cell survival rate was set arbitrarily as 100%.

Measurement of CFTR Function in HBEs: Using chamber experiments were performed on polarized airway epithelial cells expressing dF508del to assess the functional efficacy of gene-edited cells. LPC-derived HBEs were grown on Costar® Snapwell™ cell culture inserts and mounted in an Ussing chamber (Physiologic Instruments, Inc., San Diego, Calif.). The transepithelial resistance and short-circuit current in the presence of a basolateral to apical chloride gradient (Isc) were measured using a voltage-clamp system (Department of Bioengineering, University of Iowa. IA). Briefly, LPC-derived HBEs were examined under voltage-clamp recording conditions (Vhold=0 mV) at 37° C. The basolateral solution contained (in mM) 145 NaCl, 0.83 K2HPO4, 3.3 KH2PO4, 1.2 MgCl2, 1.2 CaCl2, 10 Glucose, 10 HEPES (pH adjusted to 7.35 with NaOH) and the apical solution contained (in mM) 145 NaGluconate, 1.2 MgCl2. 1.2 CaCl2, 10 glucose, 10 HEPES (pH adjusted to 7.35 with NaOH). Positive controls for CFTR function were dF508del cells treated with CFTR modulators cocktail (TC) on the basolateral side during 18-24 h prior to assay. Negative controls were cells treated with DMSO. Forskolin (10 μM) was added to the apical side during the assay to stimulate CFTR-mediated chloride transport. A CFTR-inhibitor cocktail (30 μM) was subsequently added to the apical side during the assay to inhibit CFTR-mediated chloride transport. CFTR function is expressed as μA/cm2 and it is calculated by using the following formula: Maximum Forskolin-induced current Minimal current in the presence of the CFTR inhibitor cocktail.

Nucleotide Sequences of Exemplary sgRNA and Donor Template Pairs

CFTR Intron 10 Target Site 1220
sgRNA:
(SEQ ID NO: 26)
acccagcctgacaccaaatttaGUUUUAGUACUCUGGAAACAGAAUCUACUAAAACAAGGCAA
AAUGCCGUGUUUAUCUCGUCAACUUGUUGGCGAGAUUUU, wherein
lowercase letters correspond to the nucleotide sequence comprising the sgRNA spacer and
uppercase letters correspond to the nucleotide sequence comprising the sgRNA scaffold.
Donor Template:
(SEQ ID NO: 335)
CCTGCAGGCAGCTGCGCGCTCGCTCGCTCACTGAGGCCGCCCGGGCAAAGCCCGGGC
GTCGGGCGACCTTTGGTCGCCCGGCCTCAGTGAGCGAGCGAGCGCGCAGAGAGGGAG
TGGCCAACTCCATCACTAGGGGTTCCTGCGGCCGCACGCGTATTGAGGAAATAAATTTAA
AGACATGAAAGAATCAAATTAGAGATGAGAAAGAGCTTTCTAGTATTAGAATGGGCTAAAG
GGCAATAGGTATTTGCTTCAGAAGTCTATAAAATGGTTCCTTGTTCCCATTTGATTGTCATTT
TAGCTGTGGTACTTTGTAGAAATGTGAGAAAAAGTTTAGTGGTCTCTTGAAGCTTTTCAAAA
TACTTTCTAGAATTATACCGAATAATCTAAGACAAACAGAAAAAGAAAGAGAGGAAGGAA
GAAAGAAGGAAATGAGGAAGAAAGGAAGTAGGAGGAAGGAAGGAAGGAAAGAAGGAAGG
AAGTAAGAGGGAAGCAGTGCTGCTGCTGTAGGTAAAAATGTTAATGAAAATAGAAATTAAG
AAAGACTCCTGAAAGGCAATTATTTATCAATATCTAAGATGAGGAGAACCATATTTTGAAG
AATTGAATATGAGACTTGGGAAACAAAATGCCACAAAAAATTTCCACTCAATAATATACACT
TCTGCTTAGGATGATAATTGGAGGCAAGTGAATCCTGAGCGTGATTTGATAATGACCTAATAATGAT
GGGTTTTATTTCCAGacttactgctgatggtcatcatgggcgagctggaacccagtgaggggaagatcaaacactcaggacggatttatat
gcagtcagttctcatggatcatgcctgggaccattaaggagaatatcatttttggagtgtcctacgatgaataccggtacagaagcgtgatcaaggcctgc
cagctggaggaagacattagcaagttcgcagaaaaagataacatcgtgctgggggagggcgggattactctgagtggaggccagcgggccagaatct
cactggctcgcgcagtgtacaaggacgctgatctgtatctgctggactctcccttcggctacctggacgtgctgaccgagaaagaaatcttcgagagttg
cgtctgtaagctgatggctaacaaaacccggattctggtgacatcaaagatggaacacctgaagaaagcagacaaaatcctgattctgcatgagggctca
agctacttttatgggaccttcagcgaactgcagaatctgcagcccgatttttcctctaagctgatgggatgtgactcctttgatcagttctctgccgaaaggc
gcaactccatcctgactgagaccctgcacagattcagcctggaaggcgacgctcccgtgagctggacagagactaagaaacagtcttttaagcagaca
ggcgagttcggggaaaagcgaaaaaatagcatcctgaacccaatcaatagtattcggaagttctcaatcgtgcagaaaactcccctgcagatgaacggc
attgaggaagactccgatgagccactggaacgacggctgagcctggtgcccgattccgagcagggagaagccatcctgcctaggatcagcgtcatttc
cactggcccaaccctgcaggctagaaggcgccagagtgtgctgaatctgatgacacactcagtcaaccagggccagaatatccatcggaagactaccg
cctctacaagaaaagtgagtctggctccacaggcaaacctgactgagctggacatctacagccggcggctgtcccaggagaccgggctggaaatttct
gaggaaatcaatgaggaagatctgaaggaatgctttttcgacgatatggagagtatccccgccgtgacaacttggaacacttacctgcgctatattaccgt
ccacaagtctctgatttttgtcctgatctggtgtctggtcatcttcctggctgaggtcgcagccagcctggtggtcctgtggctgctgggaaacaccccactg
caggacaaggggaattctacacatagtagaaacaatagctacgccgtgatcattacctccacaagttcatactatgtcttctacatctatgtgggcgtcgctg
atacactgctggcaatggggtttttcaggggactgcctctggtgcacacactgatcactgtctctaagattctgcaccataaaatgctgcattctgtgctgca
ggctccaatgagtaccctgaacacactgaaggcagggggaatcctgaatcggtttagcaaagacatcgccattctggacgatctgctgcctctgaccatt
tttgatttcatccagctgctgctgatcgtgattggagcaatcgctgtggtcgccgtgctgcagccttacattttcgtcgctactgtgccagtcattgtggccttc
atcatgctgcgcgcctatttcctgcagaccagccagcagctgaagcagctggagtctgaaggccggagtccaatctttacacacctggtgacttccctga
aaggactgtggaccctgagagccttcggcaggcagccctactttgagacactgttccacaaggctctgaacctgcatactgcaaattggtttctgtatctgt
ctaccctgcgatggtttcagatgcggatcgagatgattttcgtgatctttttcattgccgtcaccttcatcagcattctgaccacaggggagggagaaggca
gagtgggcatcattctgactctggccatgaacatcatgagtaccctgcagtgggctgtgaatagctccattgacgtggattcactgatgcgctcagtcagc
cgagtgtttaagttcatcgacatgcccacagaggggaagcctactaaatctaccaagccctacaaaaacggacagctgagcaaagtgatgatcattgaa
aattcccatgtcaagaaagacgacatctggcctagcggcgggcagatgaccgtgaaggatctgaccgctaaatacacagaaggaggcaacgcaattct
ggagaatatctccttttctattagtccaggacagcgagtgggactgctgggacgaacagggtcaggaaagagcactctgctgtccgcattcctgaggctg
ctgaatactgagggagaaatccagattgacggcgtgtcctgggattctatcaccctgcagcagtggagaaaggcttttggagtcatccctcagaaagtgtt
tattttcagcggcacattcaggaagaacctggacccatacgaacagtggtccgatcaggagatctggaaagtcgcagacgaagtgggactgcgctctgt
gattgaacagtttcctgggaagctggacttcgtcctggtggatgggggatgcgtgctgagccacggccataaacagctgatgtgcctggcccggagtgt
gctgtcaaaggctaaaatcctgctgctggacgagccaagcgcccacctggaccccgtgacctaccagatcattagaaggacactgaagcaggcatttg
ccgactgcaccgtgatcctgtgcgagcatcgcattgaagctatgctggagtgccagcagttcctggtcatcgaggaaaacaaggtccggcagtatgact
ctattcagaaactgctgaatgagcggagtctgtttagacaggccatctcacccagcgatagggtgaagctgttccctcaccgcaactctagtaagtgtaaa
tccaagccacagattgccgcactgaaggaagagactgaagaggaggtccaggatacaagactgtgactgactgagatacagcgtaccttcagctcaca
gacatgataagatacattgatgagtttggacaaaccacaactagaatgcagtgaaaaaaatgctttatttgtgaaatttgtgatgctattgcttta
tttgtaaccattataagctgcaataaacaagttaacaacaacaattgcattcattttatgtttcaggttcagggggaggtgtgggaggttttttAT
TTGGTGTCAGGCTGGGTGCAGTGGCTCACACTTGTAATCCTAGCACTTTTGGAGGCAGAGGC
AGGTGAATTGCTTGAGTCCAGGAGTTTGAGACCAGCGTGGGCAACATGGCAAACCCCACCT
CTACAAAAAACACAAACAAAAGAAAATAGCTGGGTGTGGTGGTGTGTGCCTGTAGTCCCAG
CTACTTGGGAGGCTGAGGTGGGAGGATCACCTGAGCCTGAGAAGTGGAGGCTGCAGTGAGC
CATGATTGCACCACTGTACCCTAGCCTAGGTGATAGGCTCAAAAAAAAAAAAAATTGGTGT
TTGCAATGCTAATAATACAATTTGGTTGTTTCTCTCTCCAGTTGTTTTCCTACATACGAAACA
GCTTTTAAAACAAAATAGCTGGAATTGTGCATTTTTTCTTACAAAAACATTTTCTTTCTTAAA
ATGTTATTATTTTTCTTTTATATCTTGTATATTATTACTAGCAGTGTTCACTATTAAAAAATTA
TAGGTAACCACGTGCGGACCGAGGCTGCAGCGTCGTCCTCCCTAGGAACCCCTAGTGATG
GAGTTGGCCACTCCCTCTCTGCGCGCTCGCTCGCTCACTGAGGCCGGGCGACCAAAGG
TCGCCCGACGCCCGGGCTTTGCCCGGGCGGCCTCAGTGAGCGAGCGAGCGCGCAGCT
GCCTGCAGG.

The uppercase boldface letters correspond to sequence comprising the AAV ITRs (SEQ ID NO: 15 for the 5′ end ITR and SEQ ID NO: 16 for the 3′ ITR); the uppercase underlined letters correspond to the nucleotide sequence comprising the 5′ and 3′ homology arms (SEQ ID NO: 17 and SEQ ID NO: 18, respectively); the uppercase italicized letters correspond to the nucleotide sequence comprising the splice site acceptor (SEQ ID NO: 1); the lowercase letters (non-boldface) correspond to the nucleotide sequence comprising CFTR exons 11-27 (SEQ ID NO: 37); and the lowercase boldface letters correspond to the nucleotide sequence comprises 3′UTR elements (SEQ ID NO: 159).

CFTR Intron 10 Target Site 2068

sgRNA:
(SEQ ID NO: 27)
tactaaaaggcagcctcctagaGUUUUAGUACUCUGGAAACAGAAUCUACUAAAACAAGGCAA
AAUGCCGUGUUUAUCUCGUCAACUUGUUGGCGAGAUUUU, wherein
lowercase letters correspond to the nucleotide sequence comprising the sgRNA spacer and
uppercase letters correspond to the nucleotide sequence comprising the sgRNA scaffold.
Donor Template:
(SEQ ID NO: 336)
CCTGCAGGCAGCTGCGCGCTCGCTCGCTCACTGAGGCCGCCCGGGCAAAGCCCGGGC
GTCGGGCGACCTTTGGTCGCCCGGCCTCAGTGAGCGAGCGAGCGCGCAGAGAGGGAG
TGGCCAACTCCATCACTAGGGGTTCCTGCGGCCGCACGCGTCAGCTTTTAAAACAAAATA
GCTGGAATTGTGCATTTTTTCTTACAAAAACATTTTCTTTCTTAAAATGTTATTATTTTTCTTT
TATATCTTGTATATTATTACTAGCAGTGTTCACTATTAAAAAATTATACTATAGGAGGGGCT
GATACTAAATAAGTTAGCAATGGTCTAAACAAGGATGTTTATTTATGAAAAGGTAGTAATTG
TGTTTCATAGAATTTTTAAAATTAATTCTGCGTATGTCTTCAAGATCAATTCTATGATAGATG
TGCAAAAATAGCTTTGGAATTACAAATTCCAAGACTTACTGGCAATTAAATTTCAGGCAGTT
TTATTAAAATTGATGAGCAGATAATTACTGGCTGACAGTGCAGTTATAGCTTATGAAAAGCA
GCTATGAAGGCAGAGTTAGAGGAAGGCAGTGGTCCCTTGGGAATATTTAAACACTTCTGAG
AAACGGAGTTTACTAACTCAATCTAGGAGGCTGCCTTTTAGTAGTTATACACTTCTGCTTAGGA
TGATAATTGGAGGCAAGTGAATCCTGAGCGTGATTTGATAATGACCTAATAATGATGGGTTTTATTT
CCAGacttctctgctgatggtcatcatgggcgagctggaacccagtgaggggaagatcaaacactcaggacggatttctttttgcagtcagttctcatg
gatcatgcctgggaccattaaggagaatatcatttttggagtgtcctacgatgaataccggtacagaagcgtgatcaaggcctgccagctggaggaagac
attagcaagttcgcagaaaaagataacatcgtgctgggggagggcgggattactctgagtggaggccagcgggccagaatctcactggctcgcgcag
tgtacaaggacgctgatctgtatctgctggactctcccttcggctacctggacgtgctgaccgagaaagaaatcttcgagagttgcgtctgtaagctgatg
gctaacaaaacccggattctggtgacatcaaagatggaacacctgaagaaagcagacaaaatcctgattctgcatgagggctcaagctacttttatggga
ccttcagcgaactgcagaatctgcagcccgatttttcctctaagctgatgggatgtgactcctttgatcagttctctgccgaaaggcgcaactccatcctgac
tgagaccctgcacagattcagcctggaaggcgacgctcccgtgagctggacagagactaagaaacagtcttttaagcagacaggcgagttcggggaa
aagcgaaaaaatagcatcctgaacccaatcaatagtattcggaagttctcaatcgtgcagaaaactcccctgcagatgaacggcattgaggaagactccg
atgagccactggaacgacggctgagcctggtgcccgattccgagcagggagaagccatcctgcctaggatcagcgtcatttccactggcccaaccctg
caggctagaaggcgccagagtgtgctgaatctgatgacacactcagtcaaccagggccagaatatccatcggaagactaccgcctctacaagaaaagt
gagtctggctccacaggcaaacctgactgagctggacatctacagccggcggctgtcccaggagaccgggctggaaatttctgaggaaatcaatgag
gaagatctgaaggaatgctttttcgacgatatggagagtatccccgccgtgacaacttggaacacttacctgcgctatattaccgtccacaagtctctgattt
ttgtcctgatctggtgtctggtcatcttcctggctgaggtcgcagccagcctggtggtcctgtggctgctgggaaacaccccactgcaggacaaggggaa
ttctacacatagtagaaacaatagctacgccgtgatcattacctccacaagttcatactatgtcttctacatctatgtgggcgtcgctgatacactgctggcaa
tggggtttttcaggggactgcctctggtgcacacactgatcactgtctctaagattctgcaccataaaatgctgcattctgtgctgcaggctccaatgagtac
cctgaacacactgaaggcagggggaatcctgaatcggtttagcaaagacatcgccattctggacgatctgctgcctctgaccatttttgatttcatccagct
gctgctgatcgtgattggagcaatcgctgtggtcgccgtgctgcagccttacattttcgtcgctactgtgccagtcattgtggccttcatcatgctgcgcgcc
tatttcctgcagaccagccagcagctgaagcagctggagtctgaaggccggagtccaatctttacacacctggtgacttccctgaaaggactgtggaccc
tgagagccttcggcaggcagccctactttgagacactgttccacaaggctctgaacctgcatactgcaaattggtttctgtatctgtctaccctgcgatggttt
cagatgcggatcgagatgattttcgtgatctttttcattgccgtcaccttcatcagcattctgaccacaggggagggagaaggcagagtgggcatcattctg
actctggccatgaacatcatgagtaccctgcagtgggctgtgaatagctccattgacgtggattcactgatgcgctcagtcagccgagtgtttaagttcatc
gacatgcccacagaggggaagcctactaaatctaccaagccctacaaaaacggacagctgagcaaagtgatgatcattgaaaattcccatgtcaagaaa
gacgacatctggcctagcggcgggcagatgaccgtgaaggatctgaccgctaaatacacagaaggaggcaacgcaattctggagaatatctccttttct
attagtccaggacagcgagtgggactgctgggacgaacagggtcaggaaagagcactctgctgtccgcattcctgaggctgctgaatactgagggag
aaatccagattgacggcgtgtcctgggattctatcaccctgcagcagtggagaaaggcttttggagtcatccctcagaaagtgtttattttcagcggcacatt
caggaagaacctggacccatacgaacagtggtccgatcaggagatctggaaagtcgcagacgaagtgggactgcgctctgtgattgaacagtttcctg
ggaagctggacttcgtcctggtggatgggggatgcgtgctgagccacggccataaacagctgatgtgcctggcccggagtgtgctgtcaaaggctaaa
atcctgctgctggacgagccaagcgcccacctggaccccgtgacctaccagatcattagaaggacactgaagcaggcatttgccgactgcaccgtgat
cctgtgcgagcatcgcattgaagctatgctggagtgccagcagttcctggtcatcgaggaaaacaaggtccggcagtatgactctattcagaaactgctg
aatgagcggagtctgtttagacaggccatctcacccagcgatagggtgaagctgttccctcaccgcaactctagtaagtgtaaatccaagccacagattg
ccgcactgaaggaagagactgaagaggaggtccaggatacaagactgtgactgactgagatacagcgtaccttcagctcacagacatgataagata
cattgatgagtttggacaaaccacaactagaatgcagtgaaaaaaatgctttatttgtgaaatttgtgatgctattgctttatttgtaaccattata
agctgcaataaacaagttaacaacaacaattgcattcattttatgtttcaggttcagggggaggtgtgggaggttttttATTAGGAATGG
AACACTTTATAGTTTTTTTTGGACAAAAGATCTAGCTAAAATATAAGATTGAATAATTGAAA
ATATTAACATTTTAAGTTAAATCTTACCCACTCAATACAATTTGGTAATTTGTATCAGAAGCT
TAAAAGATAACCTAATAGTTCTTCTACTTCTATAACTTACCCAAATATGTTTGCAGAGATCTT
ATGTAAAGCTCTTCATTATAACACTGCTTTCAGGAGCCAAAAATTGGGTGGGGGAGCCCCAT
AAATGTTGAATAATAGGGGTTTGATTAGATAAATTTTGGTGTAGTTCTATAATGGCGTGTTA
TTCAGCCAATAAAAGGTTTGTTAAAGAATGACTGTGACGGATGTATATGATATACTCTTAAG
TGAATAAAGAGTTACAAAATGTTATGTACAAGTTACAAAATGTATGTACATTATGATCCATT
TTTCATAAAATCATATGTATGTATATATGTGTGTCTGGAAGGATAAATTTATCGGTAACCAC
GTGCGGACCGAGGCTGCAGCGTCGTCCTCCCTAGGAACCCCTAGTGATGGAGTTGGCCAC
TCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAGCTGCCTGCAGG.

The uppercase boldface letters correspond to sequence comprising the AAV ITRs (SEQ ID NO: 15 for the 5′ end ITR and SEQ ID NO: 16 for the 3′ ITR); the uppercase underlined letters correspond to the nucleotide sequence comprising the 5′ and 3′ homology arms (SEQ ID NO: 19 and SEQ ID NO: 20, respectively); the uppercase italicized letters correspond to the nucleotide sequence comprising the splice site acceptor (SEQ ID NO: 1); the lowercase letters (non-boldface) correspond to the nucleotide sequence comprising CFTR exons 11-27 (SEQ ID NO: 37); and the lowercase boldface letters correspond to the nucleotide sequence comprises 3′UTR elements (SEQ ID NO: 159).

CFTR Intron 10 Target Site 3821

sgRNA:
(SEQ ID NO: 28)
attggctaccttggttggatgaGUUUUAGUACUCUGGAAACAGAAUCUACUAAAACAAGGCAAA
AUGCCGUGUUUAUCUCGUCAACUUGUUGGCGAGAUUUU, wherein
lowercase letters correspond to the nucleotide sequence comprising the sgRNA spacer and
uppercase letters correspond to the nucleotide sequence comprising the sgRNA scaffold.
Donor Template:
(SEQ ID NO: 337)
CCTGCAGGCAGCTGCGCGCTCGCTCGCTCACTGAGGCCGCCCGGGCAAAGCCCGGGC
GTCGGGCGACCTTTGGTCGCCCGGCCTCAGTGAGCGAGCGAGCGCGCAGAGAGGGAG
TGGCCAACTCCATCACTAGGGGTTCCTGCGGCCGCACGCGTTTAGAGATTAGGTCTTACT
CTGTCACCCAGGCTGAACTTCAGTGGTGTGATCATAGCTCACTGTAACCTTGAACTCCTGGG
CTCAATTGACCTTTCCGCTTCAGCCTCCCAAAGTGCTGGGTTTATAGGCATGAGCCACTGTGT
CTGGTCCAATATGCATATATATATTTTTAACCTGGATTATCAGAGCTATATTGTGTTTAGGTT
TATAAAGCTGTACTATGTGAAAATATCACTTCTAGGTTTAATTTTGTACAAAGGAATTTTATA
TAGAAATGAGGTAATTCAGATTTTTTCCCATGTAATAAGAATTGTAAAATTTACTGAAACAA
ACATCAAAAAGATATCTGTTACATGACCTTCCTTTCTTTTGAATATATTTCAGGTGATATTAT
TTATTAAAATTTAAAAATGAAAATTAAAATATATAAAAAGTTGAAAATTATTCCTTTCTTTA
CTGTCTCTCATCTGTCCATTTTCCATTCTCCTGCATTCCCTCATATACACTTCTGCTTAGGATGAT
AATTGGAGGCAAGTGAATCCTGAGCGTGATTTGATAATGACCTAATAATGATGGGTTTTATTTCCAG
acttctctgctgatggtcatcatgggcgagctggaacccagtgaggggaagatcaaacactcaggacggatttctttttgcagtcagttctcatggatcatg
cctgggaccattaaggagaatatcatttttggagtgtcctacgatgaataccggtacagaagcgtgatcaaggcctgccagctggaggaagacattagca
agttcgcagaaaaagataacatcgtgctgggggagggcgggattactctgagtggaggccagcgggccagaatctcactggctcgcgcagtgtacaa
ggacgctgatctgtatctgctggactctcccttcggctacctggacgtgctgaccgagaaagaaatcttcgagagttgcgtctgtaagctgatggctaaca
aaacccggattctggtgacatcaaagatggaacacctgaagaaagcagacaaaatcctgattctgcatgagggctcaagctacttttatgggaccttcag
cgaactgcagaatctgcagcccgatttttcctctaagctgatgggatgtgactcctttgatcagttctctgccgaaaggcgcaactccatcctgactgagac
cctgcacagattcagcctggaaggcgacgctcccgtgagctggacagagactaagaaacagtcttttaagcagacaggcgagttcggggaaaagcga
aaaaatagcatcctgaacccaatcaatagtattcggaagttctcaatcgtgcagaaaactcccctgcagatgaacggcattgaggaagactccgatgagc
cactggaacgacggctgagcctggtgcccgattccgagcagggagaagccatcctgcctaggatcagcgtcatttccactggcccaaccctgcaggct
agaaggcgccagagtgtgctgaatctgatgacacactcagtcaaccagggccagaatatccatcggaagactaccgcctctacaagaaaagtgagtct
ggctccacaggcaaacctgactgagctggacatctacagccggcggctgtcccaggagaccgggctggaaatttctgaggaaatcaatgaggaagat
ctgaaggaatgctttttcgacgatatggagagtatccccgccgtgacaacttggaacacttacctgcgctatattaccgtccacaagtctctgatttttgtcct
gatctggtgtctggtcatcttcctggctgaggtcgcagccagcctggtggtcctgtggctgctgggaaacaccccactgcaggacaaggggaattctaca
catagtagaaacaatagctacgccgtgatcattacctccacaagttcatactatgtcttctacatctatgtgggcgtcgctgatacactgctggcaatggggt
ttttcaggggactgcctctggtgcacacactgatcactgtctctaagattctgcaccataaaatgctgcattctgtgctgcaggctccaatgagtaccctgaa
cacactgaaggcagggggaatcctgaatcggtttagcaaagacatcgccattctggacgatctgctgcctctgaccatttttgatttcatccagctgctgct
gatcgtgattggagcaatcgctgtggtcgccgtgctgcagccttacattttcgtcgctactgtgccagtcattgtggccttcatcatgctgcgcgcctatttcc
tgcagaccagccagcagctgaagcagctggagtctgaaggccggagtccaatctttacacacctggtgacttccctgaaaggactgtggaccctgaga
gccttcggcaggcagccctactttgagacactgttccacaaggctctgaacctgcatactgcaaattggtttctgtatctgtctaccctgcgatggtttcagat
gcggatcgagatgattttcgtgatctttttcattgccgtcaccttcatcagcattctgaccacaggggagggagaaggcagagtgggcatcattctgactct
ggccatgaacatcatgagtaccctgcagtgggctgtgaatagctccattgacgtggattcactgatgcgctcagtcagccgagtgtttaagttcatcgacat
gcccacagaggggaagcctactaaatctaccaagccctacaaaaacggacagctgagcaaagtgatgatcattgaaaattcccatgtcaagaaagacg
acatctggcctagcggcgggcagatgaccgtgaaggatctgaccgctaaatacacagaaggaggcaacgcaattctggagaatatctccttttctattag
tccaggacagcgagtgggactgctgggacgaacagggtcaggaaagagcactctgctgtccgcattcctgaggctgctgaatactgagggagaaatc
cagattgacggcgtgtcctgggattctatcaccctgcagcagtggagaaaggcttttggagtcatccctcagaaagtgtttattttcagcggcacattcagg
aagaacctggacccatacgaacagtggtccgatcaggagatctggaaagtcgcagacgaagtgggactgcgctctgtgattgaacagtttcctgggaa
gctggacttcgtcctggtggatgggggatgcgtgctgagccacggccataaacagctgatgtgcctggcccggagtgtgctgtcaaaggctaaaatcct
gctgctggacgagccaagcgcccacctggaccccgtgacctaccagatcattagaaggacactgaagcaggcatttgccgactgcaccgtgatcctgt
gcgagcatcgcattgaagctatgctggagtgccagcagttcctggtcatcgaggaaaacaaggtccggcagtatgactctattcagaaactgctgaatga
gcggagtctgtttagacaggccatctcacccagcgatagggtgaagctgttccctcaccgcaactctagtaagtgtaaatccaagccacagattgccgca
ctgaaggaagagactgaagaggaggtccaggatacaagactgtgactgactgagatacagcgtaccttcagctcacagacatgataagatacattga
tgagtttggacaaaccacaactagaatgcagtgaaaaaaatgctttatttgtgaaatttgtgatgctattgctttatttgtaaccattataagctgc
aataaacaagttaacaacaacaattgcattcattttatgtttcaggttcagggggaggtgtgggaggttttttTCCAACCAAGGTAGC
CAATCCAGGTAACTTTTTTTAGTATCTTCCCAGAGATGTTTCTCTCTATATATATAATCAATA
TACATTTTTTATTATTCCCCACCTCTCTTTTTATGTAACAATATGCAGAGTTTTGCTTCTTGCT
TTTCCCACTATCTTGGACAACTTTCCATATTCAAAGCACAGAGGACTTGCACATATGTTCAG
ACTGCTGAATATTTCTGTCTCTCCCCTGCCATTCATATGTTGAAATCCTAATTCCCAAGGTGA
TGGTATTGCAGGGTGGGGCCTTTGGGAGGTGATTAGTCCATGAGGGTGAAGTCTTTAGTAAA
TGAGATTAGTGTCTTTATAAAAGAAACCTTAGAGAGACCCTCACACCTTAGAGAGACCCTCA
CCCCTTTCTGCCATGTGAGAACACAGCAGGAAGACAGCTGGCTATCCAGGATTCAGGAGTCT
CTTAGCAGACCCAAATCTGCTGGCACCTTGATCTTGGACTTCCCAGCGGTAACCACGTGCGG
ACCGAGGCTGCAGCGTCGTCCTCCCTAGGAACCCCTAGTGATGGAGTTGGCCACTCCCTC
TCTGCGCGCTCGCTCGCTCACTGAGGCCGGGCGACCAAAGGTCGCCCGACGCCCGGG
CTTTGCCCGGGCGGCCTCAGTGAGCGAGCGAGCGCGCAGCTGCCTGCAGG.

The uppercase boldface letters correspond to sequence comprising the AAV ITRs (SEQ ID NO: 15 for the 5′ end ITR and SEQ ID NO: 16 for the 3′ ITR); the uppercase underlined letters correspond to the nucleotide sequence comprising the 5′ and 3′ homology arms (SEQ ID NO: 21 and SEQ ID NO: 22, respectively); the uppercase italicized letters correspond to the nucleotide sequence comprising the splice site acceptor (SEQ ID NO: 1); the lowercase letters (non-boldface) correspond to the nucleotide sequence comprising CFTR exons 11-27 (SEQ ID NO: 37); and the lowercase boldface letters correspond to the nucleotide sequence comprises 3′UTR elements (SEQ ID NO: 159).

CFTR Intron 10 Target Site 4262

sgRNA:
(SEQ ID NO: 29)
gacagctggctatccaggattcGUUUUAGUACUCUGGAAACAGAAUCUACUAAAACAAGGCAA
AAUGCCGUGUUUAUCUCGUCAACUUGUUGGCGAGAUUUU, wherein
lowercase letters correspond to the nucleotide sequence comprising the sgRNA spacer and
uppercase letters correspond to the nucleotide sequence comprising the sgRNA scaffold.
Donor Template:
(SEQ ID NO: 338)
CCTGCAGGCAGCTGCGCGCTCGCTCGCTCACTGAGGCCGCCCGGGCAAAGCCCGGGC
GTCGGGCGACCTTTGGTCGCCCGGCCTCAGTGAGCGAGCGAGCGCGCAGAGAGGGAG
TGGCCAACTCCATCACTAGGGGTTCCTGCGGCCGCACGCGTTTATTCCTTTCTTTACTGTC
TCTCATCTGTCCATTTTCCATTCTCCTGCATTCCCTCATCCAACCAAGGTAGCCAATCCAGGT
AACTTTTTTTAGTATCTTCCCAGAGATGTTTCTCTCTATATATATAATCAATATACATTTTTTA
TTATTCCCCACCTCTCTTTTTATGTAACAATATGCAGAGTTTTGCTTCTTGCTTTTCCCACTAT
CTTGGACAACTTTCCATATTCAAAGCACAGAGGACTTGCACATATGTTCAGACTGCTGAATA
TTTCTGTCTCTCCCCTGCCATTCATATGTTGAAATCCTAATTCCCAAGGTGATGGTATTGCAG
GGTGGGGCCTTTGGGAGGTGATTAGTCCATGAGGGTGAAGTCTTTAGTAAATGAGATTAGTG
TCTTTATAAAAGAAACCTTAGAGAGACCCTCACACCTTAGAGAGACCCTCACCCCTTTCTGC
CATGTGAGAACACAGCAGGAAGACAGCTGGCTATCCAGGATATACACTTCTGCTTAGGATGAT
AATTGGAGGCAAGTGAATCCTGAGCGTGATTTGATAATGACCTAATAATGATGGGTTTTATTTCCAG
acttctctgctgatggtcatcatgggcgagctggaacccagtgaggggaagatcaaacactcaggacggatttctttttgcagtcagttctcatggatcatg
cctgggaccattaaggagaatatcatttttggagtgtcctacgatgaataccggtacagaagcgtgatcaaggcctgccagctggaggaagacattagca
agttcgcagaaaaagataacatcgtgctgggggagggcgggattactctgagtggaggccagcgggccagaatctcactggctcgcgcagtgtacaa
ggacgctgatctgtatctgctggactctcccttcggctacctggacgtgctgaccgagaaagaaatcttcgagagttgcgtctgtaagctgatggctaaca
aaacccggattctggtgacatcaaagatggaacacctgaagaaagcagacaaaatcctgattctgcatgagggctcaagctacttttatgggaccttcag
cgaactgcagaatctgcagcccgatttttcctctaagctgatgggatgtgactcctttgatcagttctctgccgaaaggcgcaactccatcctgactgagac
cctgcacagattcagcctggaaggcgacgctcccgtgagctggacagagactaagaaacagtcttttaagcagacaggcgagttcggggaaaagcga
aaaaatagcatcctgaacccaatcaatagtattcggaagttctcaatcgtgcagaaaactcccctgcagatgaacggcattgaggaagactccgatgagc
cactggaacgacggctgagcctggtgcccgattccgagcagggagaagccatcctgcctaggatcagcgtcatttccactggcccaaccctgcaggct
agaaggcgccagagtgtgctgaatctgatgacacactcagtcaaccagggccagaatatccatcggaagactaccgcctctacaagaaaagtgagtct
ggctccacaggcaaacctgactgagctggacatctacagccggcggctgtcccaggagaccgggctggaaatttctgaggaaatcaatgaggaagat
ctgaaggaatgctttttcgacgatatggagagtatccccgccgtgacaacttggaacacttacctgcgctatattaccgtccacaagtctctgatttttgtcct
gatctggtgtctggtcatcttcctggctgaggtcgcagccagcctggtggtcctgtggctgctgggaaacaccccactgcaggacaaggggaattctaca
catagtagaaacaatagctacgccgtgatcattacctccacaagttcatactatgtcttctacatctatgtgggcgtcgctgatacactgctggcaatggggt
ttttcaggggactgcctctggtgcacacactgatcactgtctctaagattctgcaccataaaatgctgcattctgtgctgcaggctccaatgagtaccctgaa
cacactgaaggcagggggaatcctgaatcggtttagcaaagacatcgccattctggacgatctgctgcctctgaccatttttgatttcatccagctgctgct
gatcgtgattggagcaatcgctgtggtcgccgtgctgcagccttacattttcgtcgctactgtgccagtcattgtggccttcatcatgctgcgcgcctatttcc
tgcagaccagccagcagctgaagcagctggagtctgaaggccggagtccaatctttacacacctggtgacttccctgaaaggactgtggaccctgaga
gccttcggcaggcagccctactttgagacactgttccacaaggctctgaacctgcatactgcaaattggtttctgtatctgtctaccctgcgatggtttcagat
gcggatcgagatgattttcgtgatctttttcattgccgtcaccttcatcagcattctgaccacaggggagggagaaggcagagtgggcatcattctgactct
ggccatgaacatcatgagtaccctgcagtgggctgtgaatagctccattgacgtggattcactgatgcgctcagtcagccgagtgtttaagttcatcgacat
gcccacagaggggaagcctactaaatctaccaagccctacaaaaacggacagctgagcaaagtgatgatcattgaaaattcccatgtcaagaaagacg
acatctggcctagcggcgggcagatgaccgtgaaggatctgaccgctaaatacacagaaggaggcaacgcaattctggagaatatctccttttctattag
tccaggacagcgagtgggactgctgggacgaacagggtcaggaaagagcactctgctgtccgcattcctgaggctgctgaatactgagggagaaatc
cagattgacggcgtgtcctgggattctatcaccctgcagcagtggagaaaggcttttggagtcatccctcagaaagtgtttattttcagcggcacattcagg
aagaacctggacccatacgaacagtggtccgatcaggagatctggaaagtcgcagacgaagtgggactgcgctctgtgattgaacagtttcctgggaa
gctggacttcgtcctggtggatgggggatgcgtgctgagccacggccataaacagctgatgtgcctggcccggagtgtgctgtcaaaggctaaaatcct
gctgctggacgagccaagcgcccacctggaccccgtgacctaccagatcattagaaggacactgaagcaggcatttgccgactgcaccgtgatcctgt
gcgagcatcgcattgaagctatgctggagtgccagcagttcctggtcatcgaggaaaacaaggtccggcagtatgactctattcagaaactgctgaatga
gcggagtctgtttagacaggccatctcacccagcgatagggtgaagctgttccctcaccgcaactctagtaagtgtaaatccaagccacagattgccgca
ctgaaggaagagactgaagaggaggtccaggatacaagactgtgactgactgagatacagcgtaccttcagctcacagacatgataagatacattga
tgagtttggacaaaccacaactagaatgcagtgaaaaaaatgctttatttgtgaaatttgtgatgctattgctttatttgtaaccattataagctgc
aataaacaagttaacaacaacaattgcattcattttatgtttcaggttcagggggaggtgtgggaggttttttTTCAGGAGTCTCTTA
GCAGACCCAAATCTGCTGGCACCTTGATCTTGGACTTCCCAGCCTCCAGAACTGTGAGAAAT
AAATTCCTGTTGTTTATAAGCCACACAGTTCATGGTATTTTGTTATAGCAGCCTGAACAAGG
ACACACACACACACACACACACATGCACACACATTTAAATAGATGCATAGTATTCTATCATA
TGGATGGATATTCTATGATATAATGAATCACTATTGATTGACATTTGGGTTGTTTCCAATATT
TTGTTAACACAAAGAACAACACTACAAATAACTTTATATACATATCATTTAGCACATCTGCA
ATTGTATCAGTAGGCTTCCTATAAGTGGTCAAGCATTTGTGTACTTGTGATTTTGGTAGATGT
TGTCAAATGTCCTTCCCTGAAATTTGTACCAATTCGTACTCATGCCATACACTCTAAATAGAG
TGCTGATTTCCCCACAGCATTACTAACAGATGATATTATCTAATTTAAGGTAACCACGTGCG
GACCGAGGCTGCAGCGTCGTCCTCCCTAGGAACCCCTAGTGATGGAGTTGGCCACTCCCT
CTCTGCGCGCTCGCTCGCTCACTGAGGCCGGGCGACCAAAGGTCGCCCGACGCCCGG
GCTTTGCCCGGGCGGCCTCAGTGAGCGAGCGAGCGCGCAGCTGCCTGCAGG.

The uppercase boldface letters correspond to sequence comprising the AAV ITRs (SEQ ID NO: 15 for the 5′ end ITR and SEQ ID NO: 16 for the 3′ ITR); the uppercase underlined letters correspond to the nucleotide sequence comprising the 5′ and 3′ homology arms (SEQ ID NO: 23 and SEQ ID NO: 24, respectively); the uppercase italicized letters correspond to the nucleotide sequence comprising the splice site acceptor (SEQ ID NO: 1); the lowercase letters (non-boldface) correspond to the nucleotide sequence comprising CFTR exons 11-27 (SEQ ID NO: 37); and the lowercase boldface letters correspond to the nucleotide sequence comprises 3′UTR elements (SEQ ID NO: 159).

CFTR Intron 10 Target Site 5041

sgRNA:
(SEQ ID NO: 30)
acttgcaggaggtgagggattaGUUUUAGUACUCUGGAAACAGAAUCUACUAAAACAAGGCAA
AAUGCCGUGUUUAUCUCGUCAACUUGUUGGCGAGAUUUU, wherein
lowercase letters correspond to the nucleotide sequence comprising the sgRNA spacer and
uppercase letters correspond to the nucleotide sequence comprising the sgRNA scaffold.
Donor Template:
(SEQ ID NO: 339)
CCTGCAGGCAGCTGCGCGCTCGCTCGCTCACTGAGGCCGCCCGGGCAAAGCCCGGGC
GTCGGGCGACCTTTGGTCGCCCGGCCTCAGTGAGCGAGCGAGCGCGCAGAGAGGGAG
TGGCCAACTCCATCACTAGGGGTTCCTGCGGCCGCACGCGTACAAAGAACAACACTACA
AATAACTTTATATACATATCATTTAGCACATCTGCAATTGTATCAGTAGGCTTCCTATAAGTG
GTCAAGCATTTGTGTACTTGTGATTTTGGTAGATGTTGTCAAATGTCCTTCCCTGAAATTTGT
ACCAATTCGTACTCATGCCATACACTCTAAATAGAGTGCTGATTTCCCCACAGCATTACTAA
CAGATGATATTATCTAATTTAAAAAGTTTCTCATCTTATAGGGAAAATAGTATGTCAATGTA
TTCTTAACTTGCATTTCTTTTATTATAAGTAGTGTAAAATATCATTTCAACTTATACACAGGA
GGAATTTCTCTCTATATAAAGTGATCCTAGAATCATAATGAAAAATATCACCAACTCATTAG
GAAAATGTACAAAGGATTGAATAGATATCTCATCAAAAATAAAAATATAAGTGGCCTTTAA
ACATTGAAAGGTAACATTTGAACAAAGACTTGCAGGAGGTGAGGGATATACACTTCTGCTTAG
GATGATAATTGGAGGCAAGTGAATCCTGAGCGTGATTTGATAATGACCTAATAATGATGGGTTTTAT
TTCCAGacttctctgctgatggtcatcatgggcgagctggaacccagtgaggggaagatcaaacactcaggacggatttctttttgcagtcagttctca
tggatcatgcctgggaccattaaggagaatatcatttttggagtgtcctacgatgaataccggtacagaagcgtgatcaaggcctgccagctggaggaag
acattagcaagttcgcagaaaaagataacatcgtgctgggggagggcgggattactctgagtggaggccagcgggccagaatctcactggctcgcgc
agtgtacaaggacgctgatctgtatctgctggactctcccttcggctacctggacgtgctgaccgagaaagaaatcttcgagagttgcgtctgtaagctgat
ggctaacaaaacccggattctggtgacatcaaagatggaacacctgaagaaagcagacaaaatcctgattctgcatgagggctcaagctactatatggg
accttcagcgaactgcagaatctgcagcccgatttttcctctaagctgatgggatgtgactcctttgatcagttctctgccgaaaggcgcaactccatcctga
ctgagaccctgcacagattcagcctggaaggcgacgctcccgtgagctggacagagactaagaaacagtcttttaagcagacaggcgagttcgggga
aaagcgaaaaaatagcatcctgaacccaatcaatagtattcggaagttctcaatcgtgcagaaaactcccctgcagatgaacggcattgaggaagactcc
gatgagccactggaacgacggctgagcctggtgcccgattccgagcagggagaagccatcctgcctaggatcagcgtcatttccactggcccaaccct
gcaggctagaaggcgccagagtgtgctgaatctgatgacacactcagtcaaccagggccagaatatccatcggaagactaccgcctctacaagaaaag
tgagtctggctccacaggcaaacctgactgagctggacatctacagccggcggctgtcccaggagaccgggctggaaatttctgaggaaatcaatgag
gaagatctgaaggaatgctttttcgacgatatggagagtatccccgccgtgacaacttggaacacttacctgcgctatattaccgtccacaagtctctgattt
ttgtcctgatctggtgtctggtcatcttcctggctgaggtcgcagccagcctggtggtcctgtggctgctgggaaacaccccactgcaggacaaggggaa
ttctacacatagtagaaacaatagctacgccgtgatcattacctccacaagttcatactatgtcttctacatctatgtgggcgtcgctgatacactgctggcaa
tggggtttttcaggggactgcctctggtgcacacactgatcactgtctctaagattctgcaccataaaatgctgcattctgtgctgcaggctccaatgagtac
cctgaacacactgaaggcagggggaatcctgaatcggtttagcaaagacatcgccattctggacgatctgctgcctctgaccatttttgatttcatccagct
gctgctgatcgtgattggagcaatcgctgtggtcgccgtgctgcagccttacattttcgtcgctactgtgccagtcattgtggccttcatcatgctgcgcgcc
tatttcctgcagaccagccagcagctgaagcagctggagtctgaaggccggagtccaatctttacacacctggtgacttccctgaaaggactgtggaccc
tgagagccttcggcaggcagccctactttgagacactgttccacaaggctctgaacctgcatactgcaaattggtttctgtatctgtctaccctgcgatggttt
cagatgcggatcgagatgattttcgtgatctttttcattgccgtcaccttcatcagcattctgaccacaggggagggagaaggcagagtgggcatcattctg
actctggccatgaacatcatgagtaccctgcagtgggctgtgaatagctccattgacgtggattcactgatgcgctcagtcagccgagtgtttaagttcatc
gacatgcccacagaggggaagcctactaaatctaccaagccctacaaaaacggacagctgagcaaagtgatgatcattgaaaattcccatgtcaagaaa
gacgacatctggcctagcggcgggcagatgaccgtgaaggatctgaccgctaaatacacagaaggaggcaacgcaattctggagaatatctccttttct
attagtccaggacagcgagtgggactgctgggacgaacagggtcaggaaagagcactctgctgtccgcattcctgaggctgctgaatactgagggag
aaatccagattgacggcgtgtcctgggattctatcaccctgcagcagtggagaaaggcttttggagtcatccctcagaaagtgtttattttcagcggcacatt
caggaagaacctggacccatacgaacagtggtccgatcaggagatctggaaagtcgcagacgaagtgggactgcgctctgtgattgaacagtttcctg
ggaagctggacttcgtcctggtggatgggggatgcgtgctgagccacggccataaacagctgatgtgcctggcccggagtgtgctgtcaaaggctaaa
atcctgctgctggacgagccaagcgcccacctggaccccgtgacctaccagatcattagaaggacactgaagcaggcatttgccgactgcaccgtgat
cctgtgcgagcatcgcattgaagctatgctggagtgccagcagttcctggtcatcgaggaaaacaaggtccggcagtatgactctattcagaaactgctg
aatgagcggagtctgtttagacaggccatctcacccagcgatagggtgaagctgttccctcaccgcaactctagtaagtgtaaatccaagccacagattg
ccgcactgaaggaagagactgaagaggaggtccaggatacaagactgtgactgactgagatacagcgtaccttcagctcacagacatgataagata
cattgatgagtttggacaaaccacaactagaatgcagtgaaaaaaatgctttatttgtgaaatttgtgatgctattgctttatttgtaaccattata
agctgcaataaacaagttaacaacaacaattgcattcattttatgtttcaggttcagggggaggtgtgggaggttttttTTAGGGAATGC
AGACTCTGGGAAGAGTCTTCCAAGTAGCAGGTGAAGCAAGTGCAAAGCTTTCAGATGGGAC
TGACTATACCTGTCTGGTTTGAAGAACAGTAAGGAGGTCACTGAGGCTGGCATAGAGTAAG
ACAGGGAGGGTAGAATACTGTCAGAGAAGTAATCGGCGGTGGAGGTAGGGGGTAAACCAT
AAAGTGCTCGTAAAGACTAAGGCTTATTTCTCTGGGTGAGATTAGAGGCCACTGGAGAGTTT
TAAACAGAAGTAACAGGGCCACTTTGGCTAATGTTTTTAGGCTATTCTGTAGGGAGACAAGG
GAGGAAGCAAGGAGATGAGTTAGGAGTCTATTGTGCCAGTTCAGGCAAGTGATGATGGTGG
CTTGATCCAGGTAGTAGTGGAAGTAGTATAGTAGGAAGTGATCAGATTCAGGACATGCTTTG
AAGGAAGATCCAATAGGATTAATGGATAAGTTGAACAATGGCATATGAGAAAAGTCACAGG
GTAACCACGTGCGGACCGAGGCTGCAGCGTCGTCCTCCCTAGGAACCCCTAGTGATGGAG
TTGGCCACTCCCTCTCTGCGCGCTCGCTCGCTCACTGAGGCCGGGCGACCAAAGGTCG
CCCGACGCCCGGGCTTTGCCCGGGCGGCCTCAGTGAGCGAGCGAGCGCGCAGCTGCC
TGCAGG.

The uppercase boldface letters correspond to sequence comprising the AAV ITRs (SEQ ID NO: 15 for the 5′ end ITR and SEQ ID NO: 16 for the 3′ ITR); the uppercase underlined letters correspond to the nucleotide sequence comprising the 5′ and 3′ homology arms (SEQ ID NO: 25 and SEQ ID NO: 345, respectively); the uppercase italicized letters correspond to the nucleotide sequence comprising the splice site acceptor (SEQ ID NO: 1); the lowercase letters (non-boldface) correspond to the nucleotide sequence comprising CFTR exons 11-27 (SEQ ID NO: 37); and the lowercase boldface letters correspond to the nucleotide sequence comprises 3′UTR elements (SEQ ID NO: 159).

CFTR Intron 10 Target Site 5052

sgRNA:
(SEQ ID NO: 31)
attagggaatgcagactctgggGUUUUAGUACUCUGGAAACAGAAUCUACUAAAACAAGGCAA
AAUGCCGUGUUUAUCUCGUCAACUUGUUGGCGAGAUUUU, wherein
lowercase letters correspond to the nucleotide sequence comprising the sgRNA spacer and
uppercase letters correspond to the nucleotide sequence comprising the sgRNA scaffold.
Donor Template:
(SEQ ID NO: 340)
CCTGCAGGCAGCTGCGCGCTCGCTCGCTCACTGAGGCCGCCCGGGCAAAGCCCGGGC
GTCGGGCGACCTTTGGTCGCCCGGCCTCAGTGAGCGAGCGAGCGCGCAGAGAGGGAG
TGGCCAACTCCATCACTAGGGGTTCCTGCGGCCGCACGCGTAATAACTTTATATACATAT
CATTTAGCACATCTGCAATTGTATCAGTAGGCTTCCTATAAGTGGTCAAGCATTTGTGTACTT
GTGATTTTGGTAGATGTTGTCAAATGTCCTTCCCTGAAATTTGTACCAATTCGTACTCATGCC
ATACACTCTAAATAGAGTGCTGATTTCCCCACAGCATTACTAACAGATGATATTATCTAATT
TAAAAAGTTTCTCATCTTATAGGGAAAATAGTATGTCAATGTATTCTTAACTTGCATTTCTTT
TATTATAAGTAGTGTAAAATATCATTTCAACTTATACACAGGAGGAATTTCTCTCTATATAA
AGTGATCCTAGAATCATAATGAAAAATATCACCAACTCATTAGGAAAATGTACAAAGGATT
GAATAGATATCTCATCAAAAATAAAAATATAAGTGGCCTTTAAACATTGAAAGGTAACATTT
GAACAAAGACTTGCAGGAGGTGAGGGATTAGGGAATGCAGACTCTTATACACTTCTGCTTAG
GATGATAATTGGAGGCAAGTGAATCCTGAGCGTGATTTGATAATGACCTAATAATGATGGGTTTTAT
TTCCAGacttctctgctgatggtcatcatgggcgagctggaacccagtgaggggaagatcaaacactcaggacggatttctttttgcagtcagttctca
tggatcatgcctgggaccattaaggagaatatcatttttggagtgtcctacgatgaataccggtacagaagcgtgatcaaggcctgccagctggaggaag
acattagcaagttcgcagaaaaagataacatcgtgctgggggagggcgggattactctgagtggaggccagcgggccagaatctcactggctcgcgc
agtgtacaaggacgctgatctgtatctgctggactctcccttcggctacctggacgtgctgaccgagaaagaaatcttcgagagttgcgtctgtaagctgat
ggctaacaaaacccggattctggtgacatcaaagatggaacacctgaagaaagcagacaaaatcctgattctgcatgagggctcaagctactatatggg
accttcagcgaactgcagaatctgcagcccgatttttcctctaagctgatgggatgtgactcctttgatcagttctctgccgaaaggcgcaactccatcctga
ctgagaccctgcacagattcagcctggaaggcgacgctcccgtgagctggacagagactaagaaacagtcttttaagcagacaggcgagttcgggga
aaagcgaaaaaatagcatcctgaacccaatcaatagtattcggaagttctcaatcgtgcagaaaactcccctgcagatgaacggcattgaggaagactcc
gatgagccactggaacgacggctgagcctggtgcccgattccgagcagggagaagccatcctgcctaggatcagcgtcatttccactggcccaaccct
gcaggctagaaggcgccagagtgtgctgaatctgatgacacactcagtcaaccagggccagaatatccatcggaagactaccgcctctacaagaaaag
tgagtctggctccacaggcaaacctgactgagctggacatctacagccggcggctgtcccaggagaccgggctggaaatttctgaggaaatcaatgag
gaagatctgaaggaatgctttttcgacgatatggagagtatccccgccgtgacaacttggaacacttacctgcgctatattaccgtccacaagtctctgattt
ttgtcctgatctggtgtctggtcatcttcctggctgaggtcgcagccagcctggtggtcctgtggctgctgggaaacaccccactgcaggacaaggggaa
ttctacacatagtagaaacaatagctacgccgtgatcattacctccacaagttcatactatgtcttctacatctatgtgggcgtcgctgatacactgctggcaa
tggggtttttcaggggactgcctctggtgcacacactgatcactgtctctaagattctgcaccataaaatgctgcattctgtgctgcaggctccaatgagtac
cctgaacacactgaaggcagggggaatcctgaatcggtttagcaaagacatcgccattctggacgatctgctgcctctgaccatttttgatttcatccagct
gctgctgatcgtgattggagcaatcgctgtggtcgccgtgctgcagccttacattttcgtcgctactgtgccagtcattgtggccttcatcatgctgcgcgcc
tatttcctgcagaccagccagcagctgaagcagctggagtctgaaggccggagtccaatctttacacacctggtgacttccctgaaaggactgtggaccc
tgagagccttcggcaggcagccctactttgagacactgttccacaaggctctgaacctgcatactgcaaattggtttctgtatctgtctaccctgcgatggttt
cagatgcggatcgagatgattttcgtgatctttttcattgccgtcaccttcatcagcattctgaccacaggggagggagaaggcagagtgggcatcattctg
actctggccatgaacatcatgagtaccctgcagtgggctgtgaatagctccattgacgtggattcactgatgcgctcagtcagccgagtgtttaagttcatc
gacatgcccacagaggggaagcctactaaatctaccaagccctacaaaaacggacagctgagcaaagtgatgatcattgaaaattcccatgtcaagaaa
gacgacatctggcctagcggcgggcagatgaccgtgaaggatctgaccgctaaatacacagaaggaggcaacgcaattctggagaatatctccttttct
attagtccaggacagcgagtgggactgctgggacgaacagggtcaggaaagagcactctgctgtccgcattcctgaggctgctgaatactgagggag
aaatccagattgacggcgtgtcctgggattctatcaccctgcagcagtggagaaaggcttttggagtcatccctcagaaagtgtttattttcagcggcacatt
caggaagaacctggacccatacgaacagtggtccgatcaggagatctggaaagtcgcagacgaagtgggactgcgctctgtgattgaacagtttcctg
ggaagctggacttcgtcctggtggatgggggatgcgtgctgagccacggccataaacagctgatgtgcctggcccggagtgtgctgtcaaaggctaaa
atcctgctgctggacgagccaagcgcccacctggaccccgtgacctaccagatcattagaaggacactgaagcaggcatttgccgactgcaccgtgat
cctgtgcgagcatcgcattgaagctatgctggagtgccagcagttcctggtcatcgaggaaaacaaggtccggcagtatgactctattcagaaactgctg
aatgagcggagtctgtttagacaggccatctcacccagcgatagggtgaagctgttccctcaccgcaactctagtaagtgtaaatccaagccacagattg
ccgcactgaaggaagagactgaagaggaggtccaggatacaagactgtgactgactgagatacagcgtaccttcagctcacagacatgataagata
cattgatgagtttggacaaaccacaactagaatgcagtgaaaaaaatgctttatttgtgaaatttgtgatgctattgctttatttgtaaccattata
agctgcaataaacaagttaacaacaacaattgcattcattttatgtttcaggttcagggggaggtgtgggaggttttttGGGAAGAGTCT
TCCAAGTAGCAGGTGAAGCAAGTGCAAAGCTTTCAGATGGGACTGACTATACCTGTCTGGTT
TGAAGAACAGTAAGGAGGTCACTGAGGCTGGCATAGAGTAAGACAGGGAGGGTAGAATAC
TGTCAGAGAAGTAATCGGCGGTGGAGGTAGGGGGTAAACCATAAAGTGCTCGTAAAGACTA
AGGCTTATTTCTCTGGGTGAGATTAGAGGCCACTGGAGAGTTTTAAACAGAAGTAACAGGG
CCACTTTGGCTAATGTTTTTAGGCTATTCTGTAGGGAGACAAGGGAGGAAGCAAGGAGATG
AGTTAGGAGTCTATTGTGCCAGTTCAGGCAAGTGATGATGGTGGCTTGATCCAGGTAGTAGT
GGAAGTAGTATAGTAGGAAGTGATCAGATTCAGGACATGCTTTGAAGGAAGATCCAATAGG
ATTAATGGATAAGTTGAACAATGGCATATGAGAAAAGTCACAGAGGAGTCAAAGATGATTC
GGTAACCACGTGCGGACCGAGGCTGCAGCGTCGTCCTCCCTAGGAACCCCTAGTGATGGA
GTTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGCTGC
CTGCAGG.

The uppercase boldface letters correspond to sequence comprising the AAV ITRs AAV ITRs (SEQ ID NO: 15 for the 5′ end ITR and SEQ ID NO: 16 for the 3′ ITR); the uppercase underlined letters correspond to the nucleotide sequence comprising the 5′ and 3′ homology arms (SEQ ID NO: 346 and SEQ ID NO: 347, respectively); the uppercase italicized letters correspond to the nucleotide sequence comprising the splice site acceptor (SEQ ID NO: 1); the lowercase letters (non-boldface) correspond to the nucleotide sequence comprising CFTR exons 11-27 (SEQ ID NO: 37); and the lowercase boldface letters correspond to the nucleotide sequence comprises 3′UTR elements (SEQ ID NO: 159).

CFTR Intron 10 Target Site 5278

sgRNA:
(SEQ ID NO: 32)
tgggtgagattagaggccactgGUUUUAGUACUCUGGAAACAGAAUCUACUAAAACAAGGCAA
AAUGCCGUGUUUAUCUCGUCAACUUGUUGGCGAGAUUUU, wherein
lowercase letters correspond to the nucleotide sequence comprising the sgRNA spacer and
uppercase letters correspond to the nucleotide sequence comprising the sgRNA scaffold.
Donor Template:
(SEQ ID NO: 341)
CCTGCAGGCAGCTGCGCGCTCGCTCGCTCACTGAGGCCGCCCGGGCAAAGCCCGGGC
GTCGGGCGACCTTTGGTCGCCCGGCCTCAGTGAGCGAGCGAGCGCGCAGAGAGGGAG
TGGCCAACTCCATCACTAGGGGTTCCTGCGGCCGCACGCGTTAGGGAAAATAGTATGTCA
ATGTATTCTTAACTTGCATTTCTTTTATTATAAGTAGTGTAAAATATCATTTCAACTTATACA
CAGGAGGAATTTCTCTCTATATAAAGTGATCCTAGAATCATAATGAAAAATATCACCAACTC
ATTAGGAAAATGTACAAAGGATTGAATAGATATCTCATCAAAAATAAAAATATAAGTGGCC
TTTAAACATTGAAAGGTAACATTTGAACAAAGACTTGCAGGAGGTGAGGGATTAGGGAATG
CAGACTCTGGGAAGAGTCTTCCAAGTAGCAGGTGAAGCAAGTGCAAAGCTTTCAGATGGGA
CTGACTATACCTGTCTGGTTTGAAGAACAGTAAGGAGGTCACTGAGGCTGGCATAGAGTAA
GACAGGGAGGGTAGAATACTGTCAGAGAAGTAATCGGCGGTGGAGGTAGGGGGTAAACCA
TAAAGTGCTCGTAAAGACTAAGGCTTATTTCTCTGGGTGAGATTAGAGGCCATATACACTTCT
GCTTAGGATGATAATTGGAGGCAAGTGAATCCTGAGCGTGATTTGATAATGACCTAATAATGATGG
GTTTTATTTCCAGacttctctgctgatggtcatcatgggcgagctggaacccagtgaggggaagatcaaacactcaggacggatttctttttgca
gtcagttctcatggatcatgcctgggaccattaaggagaatatcatttttggagtgtcctacgatgaataccggtacagaagcgtgatcaaggcctgccag
ctggaggaagacattagcaagttcgcagaaaaagataacatcgtgctgggggagggcgggattactctgagtggaggccagcgggccagaatctcac
tggctcgcgcagtgtacaaggacgctgatctgtatctgctggactctcccttcggctacctggacgtgctgaccgagaaagaaatcttcgagagttgcgtc
tgtaagctgatggctaacaaaacccggattctggtgacatcaaagatggaacacctgaagaaagcagacaaaatcctgattctgcatgagggctcaagct
acttttatgggaccttcagcgaactgcagaatctgcagcccgatttttcctctaagctgatgggatgtgactcctttgatcagttctctgccgaaaggcgcaa
ctccatcctgactgagaccctgcacagattcagcctggaaggcgacgctcccgtgagctggacagagactaagaaacagtcttttaagcagacaggcg
agttcggggaaaagcgaaaaaatagcatcctgaacccaatcaatagtattcggaagttctcaatcgtgcagaaaactcccctgcagatgaacggcattga
ggaagactccgatgagccactggaacgacggctgagcctggtgcccgattccgagcagggagaagccatcctgcctaggatcagcgtcatttccactg
gcccaaccctgcaggctagaaggcgccagagtgtgctgaatctgatgacacactcagtcaaccagggccagaatatccatcggaagactaccgcctct
acaagaaaagtgagtctggctccacaggcaaacctgactgagctggacatctacagccggcggctgtcccaggagaccgggctggaaatttctgagg
aaatcaatgaggaagatctgaaggaatgctttttcgacgatatggagagtatccccgccgtgacaacttggaacacttacctgcgctatattaccgtccaca
agtctctgatttttgtcctgatctggtgtctggtcatcttcctggctgaggtcgcagccagcctggtggtcctgtggctgctgggaaacaccccactgcagg
acaaggggaattctacacatagtagaaacaatagctacgccgtgatcattacctccacaagttcatactatgtcttctacatctatgtgggcgtcgctgatac
actgctggcaatggggtttttcaggggactgcctctggtgcacacactgatcactgtctctaagattctgcaccataaaatgctgcattctgtgctgcaggct
ccaatgagtaccctgaacacactgaaggcagggggaatcctgaatcggtttagcaaagacatcgccattctggacgatctgctgcctctgaccatttttga
tttcatccagctgctgctgatcgtgattggagcaatcgctgtggtcgccgtgctgcagccttacattttcgtcgctactgtgccagtcattgtggccttcatcat
gctgcgcgcctatttcctgcagaccagccagcagctgaagcagctggagtctgaaggccggagtccaatctttacacacctggtgacttccctgaaagg
actgtggaccctgagagccttcggcaggcagccctactttgagacactgttccacaaggctctgaacctgcatactgcaaattggtttctgtatctgtctacc
ctgcgatggtttcagatgcggatcgagatgattttcgtgatctttttcattgccgtcaccttcatcagcattctgaccacaggggagggagaaggcagagtg
ggcatcattctgactctggccatgaacatcatgagtaccctgcagtgggctgtgaatagctccattgacgtggattcactgatgcgctcagtcagccgagt
gtttaagttcatcgacatgcccacagaggggaagcctactaaatctaccaagccctacaaaaacggacagctgagcaaagtgatgatcattgaaaattcc
catgtcaagaaagacgacatctggcctagcggcgggcagatgaccgtgaaggatctgaccgctaaatacacagaaggaggcaacgcaattctggaga
atatctccttttctattagtccaggacagcgagtgggactgctgggacgaacagggtcaggaaagagcactctgctgtccgcattcctgaggctgctgaat
actgagggagaaatccagattgacggcgtgtcctgggattctatcaccctgcagcagtggagaaaggcttttggagtcatccctcagaaagtgtttattttc
agcggcacattcaggaagaacctggacccatacgaacagtggtccgatcaggagatctggaaagtcgcagacgaagtgggactgcgctctgtgattg
aacagtttcctgggaagctggacttcgtcctggtggatgggggatgcgtgctgagccacggccataaacagctgatgtgcctggcccggagtgtgctgt
caaaggctaaaatcctgctgctggacgagccaagcgcccacctggaccccgtgacctaccagatcattagaaggacactgaagcaggcatttgccgac
tgcaccgtgatcctgtgcgagcatcgcattgaagctatgctggagtgccagcagttcctggtcatcgaggaaaacaaggtccggcagtatgactctattc
agaaactgctgaatgagcggagtctgtttagacaggccatctcacccagcgatagggtgaagctgttccctcaccgcaactctagtaagtgtaaatccaa
gccacagattgccgcactgaaggaagagactgaagaggaggtccaggatacaagactgtgactgactgagatacagcgtaccttcagctcacagaca
tgataagatacattgatgagtttggacaaaccacaactagaatgcagtgaaaaaaatgctttatttgtgaaatttgtgatgctattgctttatttgt
aaccattataagctgcaataaacaagttaacaacaacaattgcattcattttatgtttcaggttcagggggaggtgtgggaggttttttCTGG
AGAGTTTTAAACAGAAGTAACAGGGCCACTTTGGCTAATGTTTTTAGGCTATTCTGTAGGGA
GACAAGGGAGGAAGCAAGGAGATGAGTTAGGAGTCTATTGTGCCAGTTCAGGCAAGTGATG
ATGGTGGCTTGATCCAGGTAGTAGTGGAAGTAGTATAGTAGGAAGTGATCAGATTCAGGAC
ATGCTTTGAAGGAAGATCCAATAGGATTAATGGATAAGTTGAACAATGGCATATGAGAAAA
GTCACAGAGGAGTCAAAGATGATTCCAAGCTTTCTGGACTGAGTAACTGGAAGGATAAATG
TGCCGTTTACTAGAAAGATAATGGGAGAAACAGGTTTTGGATGGAGCTTGGTTTGGGAATAT
TAAGTTTGAAATGCCTATTTGACATCCAAATAGAGATGTTAGTTGGATGTACAAGTCTAGTT
TCAAGGAAGAGGGGGCTGGTAGTGTGAAGATGGGGCTGGATAAGATTCTAAAGGAAAGAG
GGTTGAGGTAACCACGTGCGGACCGAGGCTGCAGCGTCGTCCTCCCTAGGAACCCCTAGTG
ATGGAGTTGGCCACTCCCTCTCTGCGCGCTCGCTCGCTCACTGAGGCCGGGCGACCAA
AGGTCGCCCGACGCCCGGGCTTTGCCCGGGCGGCCTCAGTGAGCGAGCGAGCGCGCA
GCTGCCTGCAGG.

The uppercase boldface letters correspond to sequence comprising the AAV ITRs (SEQ ID NO: 15 for the 5′ end ITR and SEQ ID NO: 16 for the 3′ ITR); the uppercase underlined letters correspond to the nucleotide sequence comprising the 5′ and 3′ homology arms (SEQ ID NO: 348 and SEQ ID NO: 349, respectively); the uppercase italicized letters correspond to the nucleotide sequence comprising the splice site acceptor (SEQ ID NO: 1); the lowercase letters (non-boldface) correspond to the nucleotide sequence comprising CFTR exons 11-27 (SEQ ID NO: 37); and the lowercase boldface letters correspond to the nucleotide sequence comprises 3′UTR elements (SEQ ID NO: 159).

CFTR Intron 10 Target Site 5343

sgRNA:
(SEQ ID NO: 33)
tgcttcctcccttgtctccctaGUUUUAGUACUCUGGAAACAGAAUCUACUAAAACAAGGCAAA
AUGCCGUGUUUAUCUCGUCAACUUGUUGGCGAGAUUUU, wherein
lowercase letters correspond to the nucleotide sequence comprising the sgRNA spacer and
uppercase letters correspond to the nucleotide sequence comprising the sgRNA scaffold.
Donor Template:
(SEQ ID NO: 342)
CCTGCAGGCAGCTGCGCGCTCGCTCGCTCACTGAGGCCGCCCGGGCAAAGCCCGGGC
GTCGGGCGACCTTTGGTCGCCCGGCCTCAGTGAGCGAGCGAGCGCGCAGAGAGGGAG
TGGCCAACTCCATCACTAGGGGTTCCTGCGGCCGCACGCGTTATCATTTCAACTTATACAC
AGGAGGAATTTCTCTCTATATAAAGTGATCCTAGAATCATAATGAAAAATATCACCAACTCA
TTAGGAAAATGTACAAAGGATTGAATAGATATCTCATCAAAAATAAAAATATAAGTGGCCT
TTAAACATTGAAAGGTAACATTTGAACAAAGACTTGCAGGAGGTGAGGGATTAGGGAATGC
AGACTCTGGGAAGAGTCTTCCAAGTAGCAGGTGAAGCAAGTGCAAAGCTTTCAGATGGGAC
TGACTATACCTGTCTGGTTTGAAGAACAGTAAGGAGGTCACTGAGGCTGGCATAGAGTAAG
ACAGGGAGGGTAGAATACTGTCAGAGAAGTAATCGGCGGTGGAGGTAGGGGGTAAACCAT
AAAGTGCTCGTAAAGACTAAGGCTTATTTCTCTGGGTGAGATTAGAGGCCACTGGAGAGTTT
TAAACAGAAGTAACAGGGCCACTTTGGCTAATGTTTTTAGGCTATTCTGTAGTATACACTTCT
GCTTAGGATGATAATTGGAGGCAAGTGAATCCTGAGCGTGATTTGATAATGACCTAATAATGATGG
GTTTTATTTCCAGacttctctgctgatggtcatcatgggcgagctggaacccagtgaggggaagatcaaacactcaggacggatttctttttgca
gtcagttctcatggatcatgcctgggaccattaaggagaatatcatttttggagtgtcctacgatgaataccggtacagaagcgtgatcaaggcctgccag
ctggaggaagacattagcaagttcgcagaaaaagataacatcgtgctgggggagggcgggattactctgagtggaggccagcgggccagaatctcac
tggctcgcgcagtgtacaaggacgctgatctgtatctgctggactctcccttcggctacctggacgtgctgaccgagaaagaaatcttcgagagttgcgtc
tgtaagctgatggctaacaaaacccggattctggtgacatcaaagatggaacacctgaagaaagcagacaaaatcctgattctgcatgagggctcaagct
acttttatgggaccttcagcgaactgcagaatctgcagcccgatttttcctctaagctgatgggatgtgactcctttgatcagttctctgccgaaaggcgcaa
ctccatcctgactgagaccctgcacagattcagcctggaaggcgacgctcccgtgagctggacagagactaagaaacagtcttttaagcagacaggcg
agttcggggaaaagcgaaaaaatagcatcctgaacccaatcaatagtattcggaagttctcaatcgtgcagaaaactcccctgcagatgaacggcattga
ggaagactccgatgagccactggaacgacggctgagcctggtgcccgattccgagcagggagaagccatcctgcctaggatcagcgtcatttccactg
gcccaaccctgcaggctagaaggcgccagagtgtgctgaatctgatgacacactcagtcaaccagggccagaatatccatcggaagactaccgcctct
acaagaaaagtgagtctggctccacaggcaaacctgactgagctggacatctacagccggcggctgtcccaggagaccgggctggaaatttctgagg
aaatcaatgaggaagatctgaaggaatgctttttcgacgatatggagagtatccccgccgtgacaacttggaacacttacctgcgctatattaccgtccaca
agtctctgatttttgtcctgatctggtgtctggtcatcttcctggctgaggtcgcagccagcctggtggtcctgtggctgctgggaaacaccccactgcagg
acaaggggaattctacacatagtagaaacaatagctacgccgtgatcattacctccacaagttcatactatgtcttctacatctatgtgggcgtcgctgatac
actgctggcaatggggtttttcaggggactgcctctggtgcacacactgatcactgtctctaagattctgcaccataaaatgctgcattctgtgctgcaggct
ccaatgagtaccctgaacacactgaaggcagggggaatcctgaatcggtttagcaaagacatcgccattctggacgatctgctgcctctgaccatttttga
tttcatccagctgctgctgatcgtgattggagcaatcgctgtggtcgccgtgctgcagccttacattttcgtcgctactgtgccagtcattgtggccttcatcat
gctgcgcgcctatttcctgcagaccagccagcagctgaagcagctggagtctgaaggccggagtccaatctttacacacctggtgacttccctgaaagg
actgtggaccctgagagccttcggcaggcagccctactttgagacactgttccacaaggctctgaacctgcatactgcaaattggtttctgtatctgtctacc
ctgcgatggtttcagatgcggatcgagatgattttcgtgatctttttcattgccgtcaccttcatcagcattctgaccacaggggagggagaaggcagagtg
ggcatcattctgactctggccatgaacatcatgagtaccctgcagtgggctgtgaatagctccattgacgtggattcactgatgcgctcagtcagccgagt
gtttaagttcatcgacatgcccacagaggggaagcctactaaatctaccaagccctacaaaaacggacagctgagcaaagtgatgatcattgaaaattcc
catgtcaagaaagacgacatctggcctagcggcgggcagatgaccgtgaaggatctgaccgctaaatacacagaaggaggcaacgcaattctggaga
atatctccttttctattagtccaggacagcgagtgggactgctgggacgaacagggtcaggaaagagcactctgctgtccgcattcctgaggctgctgaat
actgagggagaaatccagattgacggcgtgtcctgggattctatcaccctgcagcagtggagaaaggcttttggagtcatccctcagaaagtgtttattttc
agcggcacattcaggaagaacctggacccatacgaacagtggtccgatcaggagatctggaaagtcgcagacgaagtgggactgcgctctgtgattg
aacagtttcctgggaagctggacttcgtcctggtggatgggggatgcgtgctgagccacggccataaacagctgatgtgcctggcccggagtgtgctgt
caaaggctaaaatcctgctgctggacgagccaagcgcccacctggaccccgtgacctaccagatcattagaaggacactgaagcaggcatttgccgac
tgcaccgtgatcctgtgcgagcatcgcattgaagctatgctggagtgccagcagttcctggtcatcgaggaaaacaaggtccggcagtatgactctattc
agaaactgctgaatgagcggagtctgtttagacaggccatctcacccagcgatagggtgaagctgttccctcaccgcaactctagtaagtgtaaatccaa
gccacagattgccgcactgaaggaagagactgaagaggaggtccaggatacaagactgtgactgactgagatacagcgtaccttcagctcacagaca
tgataagatacattgatgagtttggacaaaccacaactagaatgcagtgaaaaaaatgctttatttgtgaaattttgatgctattgctttatttgt
aaccattataagctgcaataaacaagttaacaacaacaattgcattcattttatgtttcaggttcagggggaggtgtgggaggttttttGGAG
ACAAGGGAGGAAGCAAGGAGATGAGTTAGGAGTCTATTGTGCCAGTTCAGGCAAGTGATGA
TGGTGGCTTGATCCAGGTAGTAGTGGAAGTAGTATAGTAGGAAGTGATCAGATTCAGGACA
TGCTTTGAAGGAAGATCCAATAGGATTAATGGATAAGTTGAACAATGGCATATGAGAAAAG
TCACAGAGGAGTCAAAGATGATTCCAAGCTTTCTGGACTGAGTAACTGGAAGGATAAATGT
GCCGTTTACTAGAAAGATAATGGGAGAAACAGGTTTTGGATGGAGCTTGGTTTGGGAATATT
AAGTTTGAAATGCCTATTTGACATCCAAATAGAGATGTTAGTTGGATGTACAAGTCTAGTTT
CAAGGAAGAGGGGGCTGGTAGTGTGAAGATGGGGCTGGATAAGATTCTAAAGGAAAGAGG
GTTGATAAGAAGAGAAAGGGGTGTAGGGGTTAGCCTAAGGGCATTCTAAGTATTAGAGGTT
AAGGAGGGGTAACCACGTGCGGACCGAGGCTGCAGCGTCGTCCTCCCTAGGAACCCCTAG
TGATGGAGTTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGCGAGCGCG
CAGCTGCCTGCAGG.

The uppercase boldface letters correspond to sequence comprising the AAV ITRs (SEQ ID NO: 15 for the 5′ end ITR and SEQ ID NO: 16 for the 3′ ITR); the uppercase underlined letters correspond to the nucleotide sequence comprising the 5′ and 3′ homology arms (SEQ ID NO: 350 and SEQ ID NO: 351, respectively); the uppercase italicized letters correspond to the nucleotide sequence comprising the splice site acceptor (SEQ ID NO: 1); the lowercase letters (non-boldface) correspond to the nucleotide sequence comprising CFTR exons 11-27 (SEQ ID NO: 37); and the lowercase boldface letters correspond to the nucleotide sequence comprises 3′UTR elements (SEQ ID NO: 159).

CFTR Intron 10 Target Site 5538

sgRNA:
(SEQ ID NO: 34)
tggcatatgagaaaagtcacagGUUUUAGUACUCUGGAAACAGAAUCUACUAAAACAAGGCAA
AAUGCCGUGUUUAUCUCGUCAACUUGUUGGCGAGAUUUU, wherein
lowercase letters correspond to the nucleotide sequence comprising the sgRNA spacer and
uppercase letters correspond to the nucleotide sequence comprising the sgRNA scaffold.
Donor Template:
(SEQ ID NO: 343)
CCTGCAGGCAGCTGCGCGCTCGCTCGCTCACTGAGGCCGCCCGGGCAAAGCCCGGGC
GTCGGGCGACCTTTGGTCGCCCGGCCTCAGTGAGCGAGCGAGCGCGCAGAGAGGGAG
TGGCCAACTCCATCACTAGGGGTTCCTGCGGCCGCACGCGTGGATTAGGGAATGCAGACT
CTGGGAAGAGTCTTCCAAGTAGCAGGTGAAGCAAGTGCAAAGCTTTCAGATGGGACTGACT
ATACCTGTCTGGTTTGAAGAACAGTAAGGAGGTCACTGAGGCTGGCATAGAGTAAGACAGG
GAGGGTAGAATACTGTCAGAGAAGTAATCGGCGGTGGAGGTAGGGGGTAAACCATAAAGT
GCTCGTAAAGACTAAGGCTTATTTCTCTGGGTGAGATTAGAGGCCACTGGAGAGTTTTAAAC
AGAAGTAACAGGGCCACTTTGGCTAATGTTTTTAGGCTATTCTGTAGGGAGACAAGGGAGG
AAGCAAGGAGATGAGTTAGGAGTCTATTGTGCCAGTTCAGGCAAGTGATGATGGTGGCTTG
ATCCAGGTAGTAGTGGAAGTAGTATAGTAGGAAGTGATCAGATTCAGGACATGCTTTGAAG
GAAGATCCAATAGGATTAATGGATAAGTTGAACAATGGCATATGAGAAAAGTCATATACACT
TCTGCTTAGGATGATAATTGGAGGCAAGTGAATCCTGAGCGTGATTTGATAATGACCTAATAATGAT
GGGTTTTATTTCCAGacttctctgctgatggtcatcatgggcgagctggaacccagtgaggggaagatcaaacactcaggacggatttcttttt
gcagtcagttctcatggatcatgcctgggaccattaaggagaatatcatttttggagtgtcctacgatgaataccggtacagaagcgtgatcaaggcctgc
cagctggaggaagacattagcaagttcgcagaaaaagataacatcgtgctgggggagggcgggattactctgagtggaggccagcgggccagaatct
cactggctcgcgcagtgtacaaggacgctgatctgtatctgctggactctcccttcggctacctggacgtgctgaccgagaaagaaatcttcgagagttg
cgtctgtaagctgatggctaacaaaacccggattctggtgacatcaaagatggaacacctgaagaaagcagacaaaatcctgattctgcatgagggctca
agctacttttatgggaccttcagcgaactgcagaatctgcagcccgatttttcctctaagctgatgggatgtgactcctttgatcagttctctgccgaaaggc
gcaactccatcctgactgagaccctgcacagattcagcctggaaggcgacgctcccgtgagctggacagagactaagaaacagtcttttaagcagaca
ggcgagttcggggaaaagcgaaaaaatagcatcctgaacccaatcaatagtattcggaagttctcaatcgtgcagaaaactcccctgcagatgaacggc
attgaggaagactccgatgagccactggaacgacggctgagcctggtgcccgattccgagcagggagaagccatcctgcctaggatcagcgtcatttc
cactggcccaaccctgcaggctagaaggcgccagagtgtgctgaatctgatgacacactcagtcaaccagggccagaatatccatcggaagactaccg
cctctacaagaaaagtgagtctggctccacaggcaaacctgactgagctggacatctacagccggcggctgtcccaggagaccgggctggaaatttct
gaggaaatcaatgaggaagatctgaaggaatgctttttcgacgatatggagagtatccccgccgtgacaacttggaacacttacctgcgctatattaccgt
ccacaagtctctgatttttgtcctgatctggtgtctggtcatcttcctggctgaggtcgcagccagcctggtggtcctgtggctgctgggaaacaccccactg
caggacaaggggaattctacacatagtagaaacaatagctacgccgtgatcattacctccacaagttcatactatgtcttctacatctatgtgggcgtcgctg
atacactgctggcaatggggtttttcaggggactgcctctggtgcacacactgatcactgtctctaagattctgcaccataaaatgctgcattctgtgctgca
ggctccaatgagtaccctgaacacactgaaggcagggggaatcctgaatcggtttagcaaagacatcgccattctggacgatctgctgcctctgaccatt
tttgatttcatccagctgctgctgatcgtgattggagcaatcgctgtggtcgccgtgctgcagccttacattttcgtcgctactgtgccagtcattgtggccttc
atcatgctgcgcgcctatttcctgcagaccagccagcagctgaagcagctggagtctgaaggccggagtccaatctttacacacctggtgacttccctga
aaggactgtggaccctgagagccttcggcaggcagccctactttgagacactgttccacaaggctctgaacctgcatactgcaaattggtttctgtatctgt
ctaccctgcgatggtttcagatgcggatcgagatgattttcgtgatctttttcattgccgtcaccttcatcagcattctgaccacaggggagggagaaggca
gagtgggcatcattctgactctggccatgaacatcatgagtaccctgcagtgggctgtgaatagctccattgacgtggattcactgatgcgctcagtcagc
cgagtgtttaagttcatcgacatgcccacagaggggaagcctactaaatctaccaagccctacaaaaacggacagctgagcaaagtgatgatcattgaa
aattcccatgtcaagaaagacgacatctggcctagcggcgggcagatgaccgtgaaggatctgaccgctaaatacacagaaggaggcaacgcaattct
ggagaatatctccttttctattagtccaggacagcgagtgggactgctgggacgaacagggtcaggaaagagcactctgctgtccgcattcctgaggctg
ctgaatactgagggagaaatccagattgacggcgtgtcctgggattctatcaccctgcagcagtggagaaaggcttttggagtcatccctcagaaagtgtt
tattttcagcggcacattcaggaagaacctggacccatacgaacagtggtccgatcaggagatctggaaagtcgcagacgaagtgggactgcgctctgt
gattgaacagtttcctgggaagctggacttcgtcctggtggatgggggatgcgtgctgagccacggccataaacagctgatgtgcctggcccggagtgt
gctgtcaaaggctaaaatcctgctgctggacgagccaagcgcccacctggaccccgtgacctaccagatcattagaaggacactgaagcaggcatttg
ccgactgcaccgtgatcctgtgcgagcatcgcattgaagctatgctggagtgccagcagttcctggtcatcgaggaaaacaaggtccggcagtatgact
ctattcagaaactgctgaatgagcggagtctgtttagacaggccatctcacccagcgatagggtgaagctgttccctcaccgcaactctagtaagtgtaaa
tccaagccacagattgccgcactgaaggaagagactgaagaggaggtccaggatacaagactgtgactgactgagatacagcgtaccttcagctcaca
gacatgataagatacattgatgagtttggacaaaccacaactagaatgcagtgaaaaaaatgctttatttgtgaaatttgtgatgctattgcttta
tttgtaaccattataagctgcaataaacaagttaacaacaacaattgcattcattttatgtttcaggttcagggggaggtgtgggaggttttttCA
GAGGAGTCAAAGATGATTCCAAGCTTTCTGGACTGAGTAACTGGAAGGATAAATGTGCCGT
TTACTAGAAAGATAATGGGAGAAACAGGTTTTGGATGGAGCTTGGTTTGGGAATATTAAGTT
TGAAATGCCTATTTGACATCCAAATAGAGATGTTAGTTGGATGTACAAGTCTAGTTTCAAGG
AAGAGGGGGCTGGTAGTGTGAAGATGGGGCTGGATAAGATTCTAAAGGAAAGAGGGTTGA
TAAGAAGAGAAAGGGGTGTAGGGGTTAGCCTAAGGGCATTCTAAGTATTAGAGGTTAAGGA
GGTGGGTGAAGAAAACCCAATAAAATAAAAGTCTGAGAAGACAAAGCTAGTGAATGAATG
TGGTATCCCGGAACCCAACTGATGTCAAGCAGAAGGGTGTTATCAACTAGGTCAAATGCTC
ATTCATCAAGTAAGATGAAACTGTTATAATTAACCGGTGTCTTCTGAAATACGGAGATAACT
CGTGACTTAGGTAACCACGTGCGGACCGAGGCTGCAGCGTCGTCCTCCCTAGGAACCCCTA
GTGATGGAGTTGGCCACTCCCTCTCTGCGCGCTCGCTCGCTCACTGAGGCCGGGCGAC
CAAAGGTCGCCCGACGCCCGGGCTTTGCCCGGGCGGCCTCAGTGAGCGAGCGAGCGC
GCAGCTGCCTGCAGG.

The uppercase boldface letters correspond to sequence comprising the AAV ITRs (SEQ ID NO: 15 for the 5′ end ITR and SEQ ID NO: 16 for the 3′ ITR); the uppercase underlined letters correspond to the nucleotide sequence comprising the 5′ and 3′ homology arms (SEQ ID NO: 352 and SEQ ID NO: 353, respectively); the uppercase italicized letters correspond to the nucleotide sequence comprising the splice site acceptor (SEQ ID NO: 1); the lowercase letters (non-boldface) correspond to the nucleotide sequence comprising CFTR exons 11-27 (SEQ ID NO: 37); and the lowercase boldface letters correspond to the nucleotide sequence comprises 3′UTR elements (SEQ ID NO: 159).

CFTR Intron 10 Target Site 6150

sgRNA:
(SEQ ID NO: 35)
ccttattcttttgatatactccGUUUUAGUACUCUGGAAACAGAAUCUACUAAAACAAGGCAAAA
UGCCGUGUUUAUCUCGUCAACUUGUUGGCGAGAUUUU, wherein
lowercase letters correspond to the nucleotide sequence comprising the sgRNA spacer and
uppercase letters correspond to the nucleotide sequence comprising the sgRNA scaffold.
Donor Template:
(SEQ ID NO: 344)
CCTGCAGGCAGCTGCGCGCTCGCTCGCTCACTGAGGCCGCCCGGGCAAAGCCCGGGC
GTCGGGCGACCTTTGGTCGCCCGGCCTCAGTGAGCGAGCGAGCGCGCAGAGAGGGAG
TGGCCAACTCCATCACTAGGGGTTCCTGCGGCCGCACGCGTGGAATATTAAGTTTGAAAT
GCCTATTTGACATCCAAATAGAGATGTTAGTTGGATGTACAAGTCTAGTTTCAAGGAAGAGG
GGGCTGGTAGTGTGAAGATGGGGCTGGATAAGATTCTAAAGGAAAGAGGGTTGATAAGAA
GAGAAAGGGGTGTAGGGGTTAGCCTAAGGGCATTCTAAGTATTAGAGGTTAAGGAGGTGGG
TGAAGAAAACCCAATAAAATAAAAGTCTGAGAAGACAAAGCTAGTGAATGAATGTGGTATC
CCGGAACCCAACTGATGTCAAGCAGAAGGGTGTTATCAACTAGGTCAAATGCTCATTCATCA
AGTAAGATGAAACTGTTATAATTAACCGGTGTCTTCTGAAATACGGAGATAACTCGTGACTT
AATGAAAGCAATAGTAGAGAAGGTCAAACTTGACCAGAATGAAATTAGAAAGAATAAGAG
GAAAGAAAAGACCAAATACAGACAACCATTGATGCCTTATTCTTTTGATATACTATACACTTC
TGCTTAGGATGATAATTGGAGGCAAGTGAATCCTGAGCGTGATTTGATAATGACCTAATAATGATGG
GTTTTATTTCCAGacttctctgctgatggtcatcatgggcgagctggaacccagtgaggggaagatcaaacactcaggacggatttctttttgca
gtcagttctcatggatcatgcctgggaccattaaggagaatatcatttttggagtgtcctacgatgaataccggtacagaagcgtgatcaaggcctgccag
ctggaggaagacattagcaagttcgcagaaaaagataacatcgtgctgggggagggcgggattactctgagtggaggccagcgggccagaatctcac
tggctcgcgcagtgtacaaggacgctgatctgtatctgctggactctcccttcggctacctggacgtgctgaccgagaaagaaatcttcgagagttgcgtc
tgtaagctgatggctaacaaaacccggattctggtgacatcaaagatggaacacctgaagaaagcagacaaaatcctgattctgcatgagggctcaagct
acttttatgggaccttcagcgaactgcagaatctgcagcccgatttttcctctaagctgatgggatgtgactcctttgatcagttctctgccgaaaggcgcaa
ctccatcctgactgagaccctgcacagattcagcctggaaggcgacgctcccgtgagctggacagagactaagaaacagtcttttaagcagacaggcg
agttcggggaaaagcgaaaaaatagcatcctgaacccaatcaatagtattcggaagttctcaatcgtgcagaaaactcccctgcagatgaacggcattga
ggaagactccgatgagccactggaacgacggctgagcctggtgcccgattccgagcagggagaagccatcctgcctaggatcagcgtcatttccactg
gcccaaccctgcaggctagaaggcgccagagtgtgctgaatctgatgacacactcagtcaaccagggccagaatatccatcggaagactaccgcctct
acaagaaaagtgagtctggctccacaggcaaacctgactgagctggacatctacagccggcggctgtcccaggagaccgggctggaaatttctgagg
aaatcaatgaggaagatctgaaggaatgctttttcgacgatatggagagtatccccgccgtgacaacttggaacacttacctgcgctatattaccgtccaca
agtctctgatttttgtcctgatctggtgtctggtcatcttcctggctgaggtcgcagccagcctggtggtcctgtggctgctgggaaacaccccactgcagg
acaaggggaattctacacatagtagaaacaatagctacgccgtgatcattacctccacaagttcatactatgtcttctacatctatgtgggcgtcgctgatac
actgctggcaatggggtttttcaggggactgcctctggtgcacacactgatcactgtctctaagattctgcaccataaaatgctgcattctgtgctgcaggct
ccaatgagtaccctgaacacactgaaggcagggggaatcctgaatcggtttagcaaagacatcgccattctggacgatctgctgcctctgaccatttttga
tttcatccagctgctgctgatcgtgattggagcaatcgctgtggtcgccgtgctgcagccttacattttcgtcgctactgtgccagtcattgtggccttcatcat
gctgcgcgcctatttcctgcagaccagccagcagctgaagcagctggagtctgaaggccggagtccaatctttacacacctggtgacttccctgaaagg
actgtggaccctgagagccttcggcaggcagccctactttgagacactgttccacaaggctctgaacctgcatactgcaaattggtttctgtatctgtctacc
ctgcgatggtttcagatgcggatcgagatgattttcgtgatctttttcattgccgtcaccttcatcagcattctgaccacaggggagggagaaggcagagtg
ggcatcattctgactctggccatgaacatcatgagtaccctgcagtgggctgtgaatagctccattgacgtggattcactgatgcgctcagtcagccgagt
gtttaagttcatcgacatgcccacagaggggaagcctactaaatctaccaagccctacaaaaacggacagctgagcaaagtgatgatcattgaaaattcc
catgtcaagaaagacgacatctggcctagcggcgggcagatgaccgtgaaggatctgaccgctaaatacacagaaggaggcaacgcaattctggaga
atatctccttttctattagtccaggacagcgagtgggactgctgggacgaacagggtcaggaaagagcactctgctgtccgcattcctgaggctgctgaat
actgagggagaaatccagattgacggcgtgtcctgggattctatcaccctgcagcagtggagaaaggcttttggagtcatccctcagaaagtgtttattttc
agcggcacattcaggaagaacctggacccatacgaacagtggtccgatcaggagatctggaaagtcgcagacgaagtgggactgcgctctgtgattg
aacagtttcctgggaagctggacttcgtcctggtggatgggggatgcgtgctgagccacggccataaacagctgatgtgcctggcccggagtgtgctgt
caaaggctaaaatcctgctgctggacgagccaagcgcccacctggaccccgtgacctaccagatcattagaaggacactgaagcaggcatttgccgac
tgcaccgtgatcctgtgcgagcatcgcattgaagctatgctggagtgccagcagttcctggtcatcgaggaaaacaaggtccggcagtatgactctattc
agaaactgctgaatgagcggagtctgtttagacaggccatctcacccagcgatagggtgaagctgttccctcaccgcaactctagtaagtgtaaatccaa
gccacagattgccgcactgaaggaagagactgaagaggaggtccaggatacaagactgtgactgactgagatacagcgtaccttcagctcacagaca
tgataagatacattgatgagtttggacaaaccacaactagaatgcagtgaaaaaaatgctttatttgtgaaatttgtgatgctattgctttatttgt
aaccattataagctgcaataaacaagttaacaacaacaattgcattcattttatgtttcaggttcagggggaggtgtgggaggttttttTCCTG
GAGTCCACTTGCTAATACAATTGACCCTTAAACAATACAGGCTTGAACTGCATGGGTCCACT
TATTTGTGAATTTTTTTTCAGTTAATACATTGGAAAATTTTTGGGGTTTTTTGACAATTTGAA
AAAACTCACAAACTGTCTAGCCTAGAAATACCGAGAAAATTAAGAAAAAGTAAGATATGCC
ATGAATGCATAAAATATATGTAGACACTAGCCTATTTTATCATTTGCTACTATAAAATATAC
ACAATCTATTATAAAAAGTTAAAATTTATCAAAACTTAACACACACTAACACCTACCCTACC
TGGCACCATTCACAGTAAAGAGAAATGTAAATAAACATAAAAATGTAGTATTAAACCATAA
TGGCATAAAACTAATTGTAGTACATATGGTACTACTGTAATAATTTGGAAGCCACTTCCTGT
TGCTATTACGGTAAGCTCAAGCATTGTGGATAGCCATTTAAAACACCACGTGATGCTAATCA
GGTAACCACGTGCGGACCGAGGCTGCAGCGTCGTCCTCCCTAGGAACCCCTAGTGATGGA
GTTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGCTGC
CTGCAGG.

The uppercase boldface letters correspond to sequence comprising the AAV ITRs (SEQ ID NO: 15 for the 5′ end ITR and SEQ ID NO: 16 for the 3′ ITR); the uppercase underlined letters correspond to the nucleotide sequence comprising the 5′ and 3′ homology arms (SEQ ID NO: 354 and SEQ ID NO: 355, respectively); the uppercase italicized letters correspond to the nucleotide sequence comprising the splice site acceptor (SEQ ID NO: 1); the lowercase letters (non-boldface) correspond to the nucleotide sequence comprising CFTR exons 11-27 (SEQ ID NO: 37); and the lowercase boldface letters correspond to the nucleotide sequence comprises 3′UTR elements (SEQ ID NO: 159).

Results

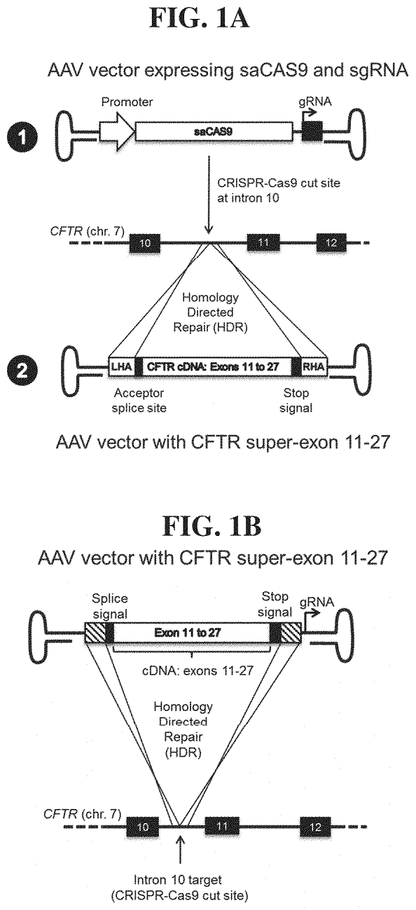

Design of dual AAV gene-editing systems for correcting mutations of the CFTR gene: FIG. 1 shows a schematic depicts exemplary CFTR gene-editing strategies. More than 99% of Cystic Fibrosis-causing mutations are located between exon 11 and 27 of the CFTR gene. AAV can provide a single-stranded DNA donor for an efficient DNA insertion mediated by homology directed repair (HDR) at a CRISPR-Cas9 cutting site. The CFTR super-exon AAV donor sets were designed to contain left and right homology arms from selected CRISPR-Cas9 cut sites at CFTR intron 10 (LHA and RHA), a cDNA comprising exon 11 until exon 27 of wild type CFTR gene, and an acceptor splice site and stop signal. FIG. 1A depicts a strategy based on two AAV vectors. The first AAV vector expresses saCAS9 and an sgRNA to induce a specific double-strand DNA cut at intron 10 of CFTR gene, and the second AAV vector serves as a HDR donor template. FIG. 1B depicts a strategy based on a single AAV vector.

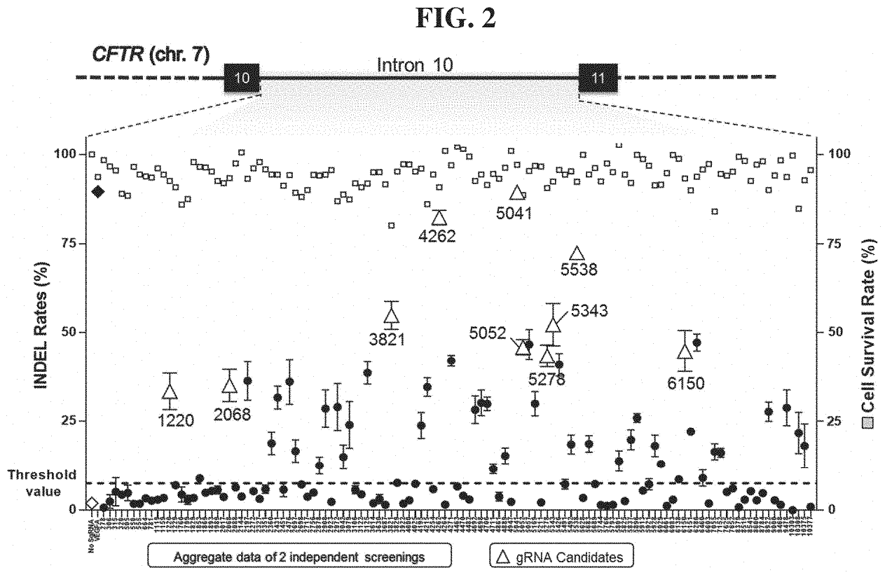

Identification of effective saCAS9-gRNAs gene-editing complexes in CFTR intron 10: Lung progenitor cells (LPCs) were electroporated with saCAS9 mRNA together with a sgRNA targeting a site located in CFTR intron (see FIG. 2). Positive and negative controls were cells electroporated with saCAS9 mRNA together VEGFA gRNA or without gRNA, respectively. Indel rates were determined by using the TIDE assay 72 hours after electroporation. gRNAs with indel rates above threshold value were considered as active gRNA sites. The threshold value was set as 7.5% indel rate (4 SD values above the mean of negative control). Cell survival rates are shown in percentages where mock electroporated cells were set arbitrarily as 100%. Indel and cell survival rates in FIG. 2 represent the average values of 2 independent experiments (n=3). 10 candidate gRNAs were selected based on Indel rates, cell survival rates, and location in CFTR intron 10 (white triangles). See TABLE 1 for sgRNA sequences tested in this study together INDEL and cell survival rates.

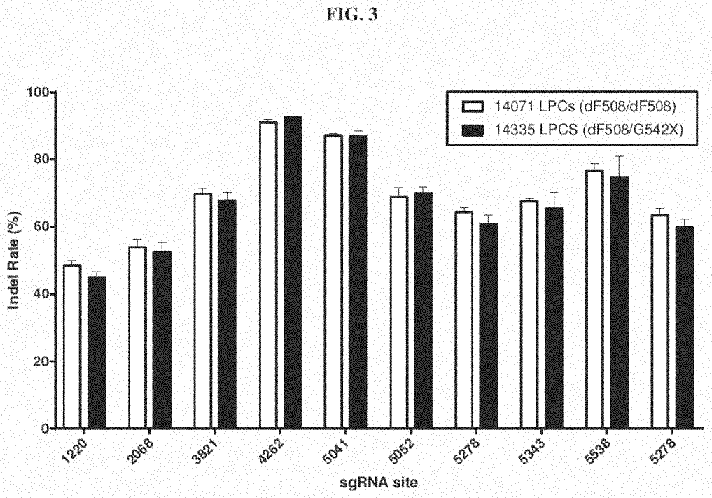

LPCs derived from two Cystic Fibrosis donors (14071 and 14335) were then electroporated with saCAS9 mRNA together with a gRNA targeting one of the 10 candidate CFTR intron 10 target sites. Indel rates were determined by using TIDE assay 72 h after electroporation. There were no significant differences in Indel rates between LPC donors for each of the 10 candidate sgRNA target sites (FIG. 3).

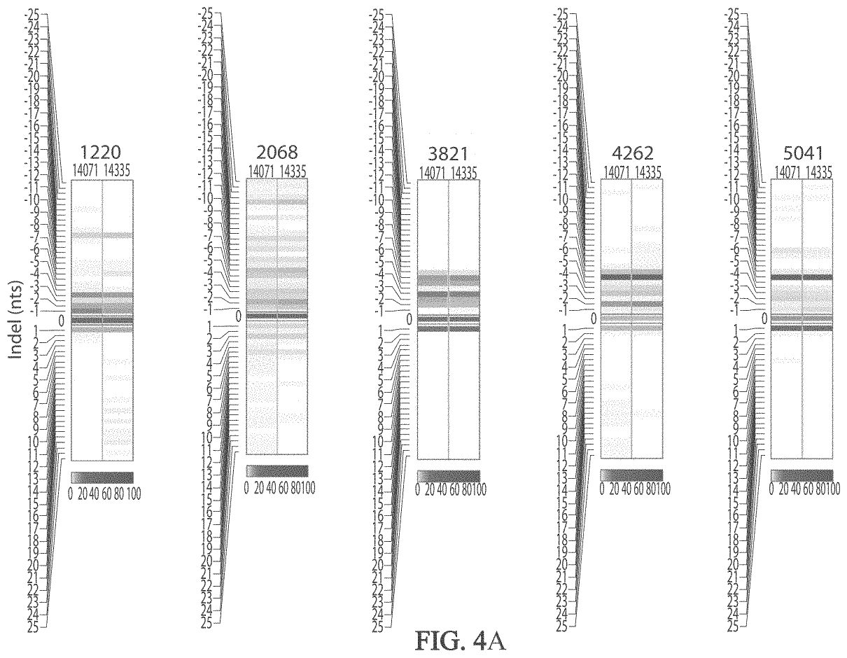

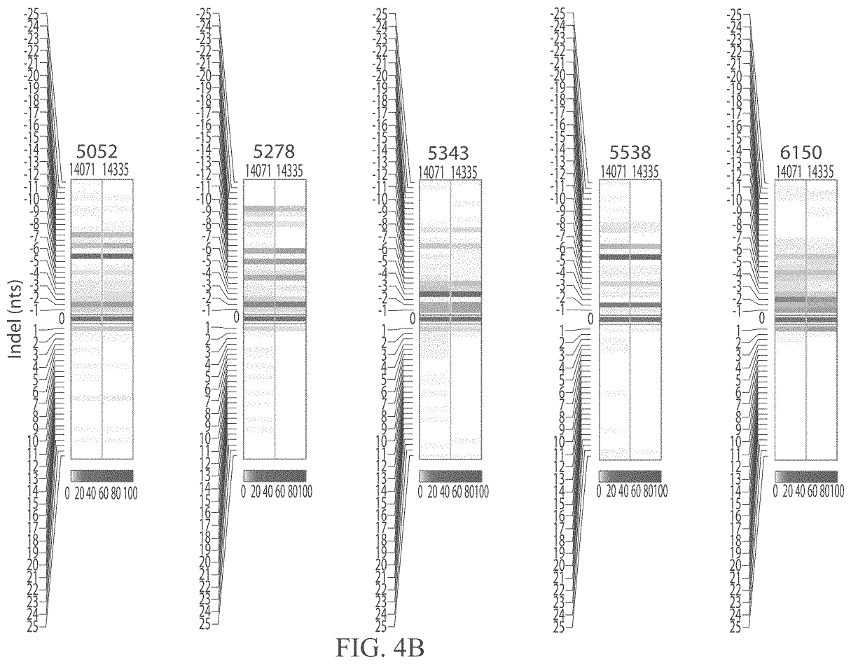

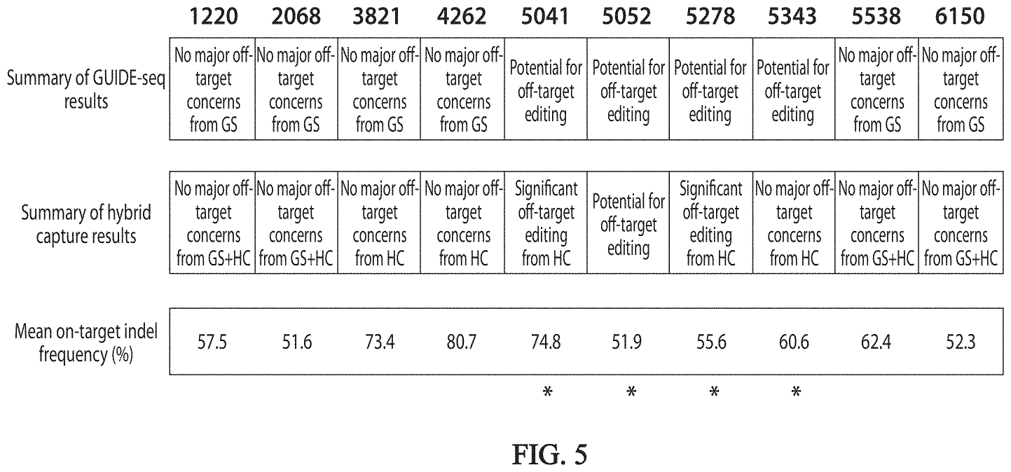

Determination of Indel pattern consistency of the 10 candidate sgRNA target site: Indel patterns for the 10 candidate sgRNA target sites (identified and labeled in FIG. 2) were determined in LPCs from two independent donors. Indel rates were determined by using TIDE assay 72 h after electroporation. There were no significant differences in indel rates between LPC donors for each of the 10 candidate selected gRNAs (FIG. 4).

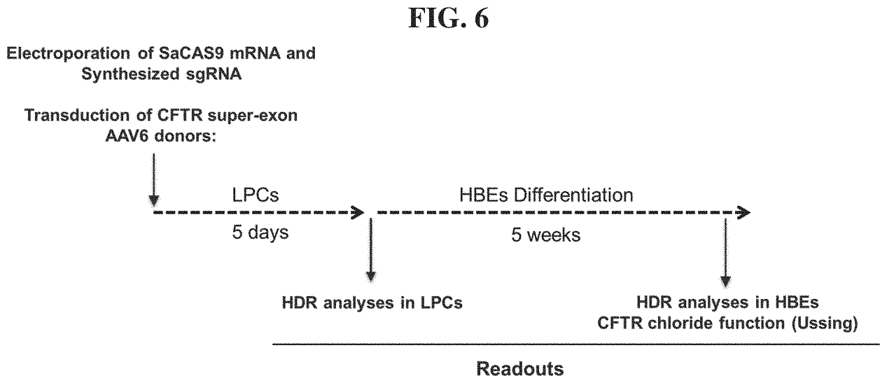

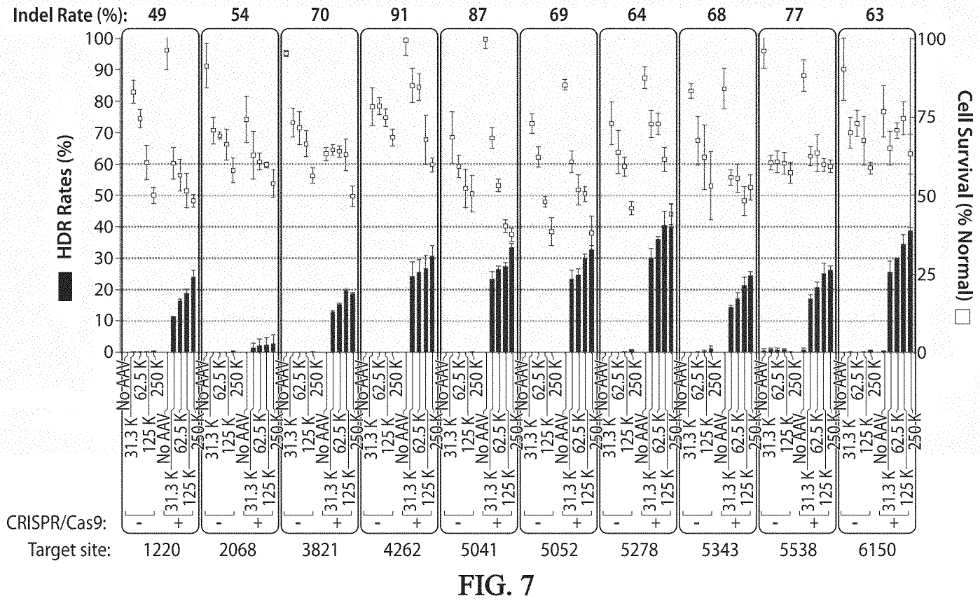

Determination of the rates of CFTR super-exon insertion by HDR in LPC: LPCs were electroporated with saCAS9 mRNA together with an sgRNA targeting one of the 10 candidate target sites. LPC cells were seeded with media containing CFTR super-exon AAV vectors. Homology dependent recombination (HDR) rates were measured by ddPCR after 5 days of treatment in LPCs and after 5 weeks of LPC differentiation into HBEs. CFTR function was measured in 5 weeks differentiated HBEs by Ussing assay (see FIG. 6). Rates of homology-dependent recombination (HDR) of CFTR super-exon 11-27 and cell survival corresponding to the 10 candidate gRNA target sites in LPCs are shown in FIG. 7.

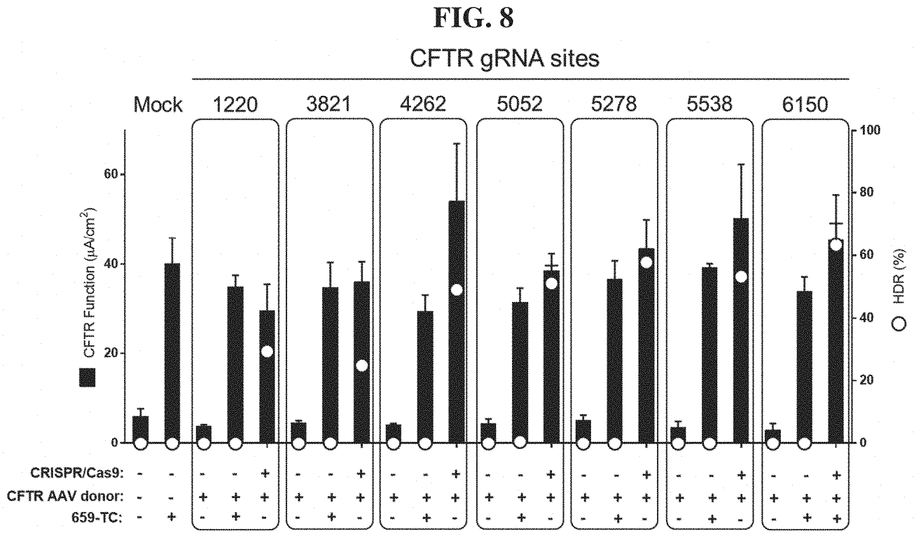

Determination of functional CFTR correction in HBEs derived from gene-edited LPCs: dF508/dF508 LPCs were electroporated with or without saCAS9 mRNA and an sgRNA targeting one of the 10 candidate target sites. LPC cells were seeded with media containing CFTR super-exon AAV vectors. Homology dependent recombination (HDR) rates were measured by ddPCR after 5 days of treatment in LPCs and after 5 weeks of LPC differentiation into HBEs. CFTR function was measured in 5 weeks differentiated HBEs by Ussing assay (see FIG. 6). Rates of homology-dependent recombination (HDR) of CFTR super-exon 11-27 and functional correction corresponding to the 10 candidate gRNA target sites in LPCs are shown in FIG. 8.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one of skill in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A gene-editing system for modifying a cystic fibrosis transmembrane regulator (CFTR) gene, the gene-editing system comprising:
(a) a first polynucleotide, which comprises a first nucleotide sequence encoding exons 11 to 27 of the CFTR gene;
(b) a second polynucleotide, which comprises a second nucleotide sequence encoding an RNA-guided DNA endonuclease, or the RNA-guided DNA endonuclease; and
(c) a third polynucleotide, which comprises a third nucleotide sequence encoding a guide RNA (gRNA), wherein the gRNA directs cleavage by the RNA-guided DNA endonuclease at a target site, which is position 1220, 2068, 3821, 4262, 5041, 5052, 5278, 5343, 5538, or 6150 of intron 10 in the CFTR gene.

2. The gene-editing system of claim 1, wherein the first nucleotide sequence is free of intron sequences.

3. The gene-editing system of claim 1, wherein the first polynucleotide of (a) further comprises a 5′ homologous arm upstream to the first nucleotide sequence and a 3′ homologous arm downstream to the first nucleotide sequence, wherein the 5′ homologous arm comprises a nucleic acid sequence that is homologous to a region upstream to the target site, and wherein the 3′ homologous arm comprises a nucleic acid sequence that is homologous to a region downstream to the target site.

4. The gene-editing system of claim 3, wherein the 5′ homologous arm and the 3′ homologous arm comprise nucleotide sequences selected from the group consisting of:
(i) SEQ ID NO: 17 and SEQ ID NO: 18, respectively;
(ii) SEQ ID NO: 19 and SEQ ID NO: 20, respectively;
(iii) SEQ ID NO: 21 and SEQ ID NO: 22, respectively;
(iv) SEQ ID NO: 23 and SEQ ID NO: 24, respectively;
(v) SEQ ID NO: 25 and SEQ ID NO: 345, respectively;
(vi) SEQ ID NO: 346 and SEQ ID NO: 347, respectively;
(vii) SEQ ID NO: 348 and SEQ ID NO: 349, respectively;
(viii) SEQ ID NO: 350 and SEQ ID NO: 351, respectively;
(ix) SEQ ID NO: 352 and SEQ ID NO: 353, respectively; and
(x) SEQ ID NO: 354 and SEQ ID NO: 355, respectively.

5. The gene-editing system of claim 1, wherein the first nucleotide sequence in (a) further comprises a first fragment upstream to the first nucleotide sequence and downstream to the 5′ homologous arm, and wherein the first fragment contains an acceptor splice site.

6. (canceled)

7. The gene-editing system of claim 1, wherein the second nucleotide sequence encoding the RNA-guided DNA endonuclease further comprises a nucleotide sequence encoding a nuclear localization signal (NLS), which is fused in-frame with the RNA-guided DNA endonuclease.

8. (canceled)

9. The gene-editing system of claim 1, wherein the RNA-guided DNA endonuclease is a Cas9 endonuclease.

10. (canceled)

11. The gene-editing system of claim 1, wherein the third nucleotide sequence in (c), which encodes the gRNA, comprises one of the following:
(i)
(SEQ ID NO: 2)
ACCCAGCCTGACACCAAATTTA
or
(SEQ ID NO: 51
ACCCAGCCUGACACCAAAUUUA;
(ii)
(SEQ ID NO: 3)
TACTAAAAGGCAGCCTCCTAGA
or
(SEQ ID NO: 61
UACUAAAAGGCAGCCUCCUAGA;
(iii)
(SEQ ID NO: 4)
ATTGGCTACCTTGGTTGGATGA
or
(SEQ ID NO: 88)
AUUGGCUACCUUGGUUGGAUGA;
(iv)
(SEQ ID NO: 5)
GACAGCTGGCTATCCAGGATTC
or
(SEQ ID NO: 96)
GACAGCUGGCUAUCCAGGAUUC;
(v)
(SEQ ID NO: 6)
ACTTGCAGGAGGTGAGGGATTA
or
(SEQ ID NO: 109)
ACUUGCAGGAGGUGAGGGAUUA;
(vi)
(SEQ ID NO: 7)
ATTAGGGAATGCAGACTCTGGG
or
(SEQ ID NO: 110)
AUUAGGGAAUGCAGACUCUGGG;
(vii)
(SEQ ID NO: 8)
TGGGTGAGATTAGAGGCCACTG
or
(SEQ ID NO: 114)
UGGGUGAGAUUAGAGGCCACUG;
(viii)
(SEQ ID NO: 9)
TGCTTCCTCCCTTGTCTCCCTA
or
(SEQ ID NO: 115)
UGCUUCCUCCCUUGUCUCCCUA;
(iv)
(SEQ ID NO: 10)
TGGCATATGAGAAAAGTCACAG
or
(SEQ ID NO: 119)
UGGCAUAUGAGAAAAGUCACAG;
and
(x)
(SEQ ID NO: 11)
CCTTATTCTTTTGATATACTCC
or
(SEQ ID NO: 137)
CCUUAUUCUUUUGAUAUACUCC.

12. The gene-editing system of claim 11, wherein the third nucleotide sequence in (c) further comprises a scaffold sequence.

13. (canceled)

14. The gene-editing system of claim 1, wherein (a), (b), and (c) are located on the same vector or on different vectors.

15-18. (canceled)

19. A viral particle or a set of viral particles, which collectively comprises the gene-editing system of claim 1.

20. (canceled)

21. A method of editing a cystic fibrosis transmembrane regulator (CFTR) gene, the method comprises contacting a cell with a gene-editing system of claim 1.

22-23. (canceled)

24. The method of claim 21, wherein the contacting step is performed by administering the gene-editing system to a subject in need thereof.

25. The method of claim 24, wherein the gene-editing system is administered to the respiratory tract of the subject.

26-27. (canceled)

28. The method of claim 21, wherein the cell is a stem cell.

29. (canceled)

30. The method of claim 28, wherein the method further comprises administering the cell with the edited CFTR gene to a subject in need thereof.

31. (canceled)

32. The method of claim 30, wherein the subject is a human patient having cystic fibrosis.

33. (canceled)

34. A nucleic acid comprising:
(a) a first nucleotide sequence encoding exons 11 to 27 of a cystic fibrosis transmembrane conductance regulator (CFTR) gene;
(b) a 5′ homologous arm upstream to the first nucleotide sequence, wherein the 5′ homologous arm comprises a nucleic acid sequence that is homologous to a region upstream to a target position in intron 10 of the CFTR gene; and
(c) a 3′ homologous arm downstream to the first nucleotide sequence, wherein the 3′ homologous arm comprises a nucleic acid sequence that is homologous to a region downstream to a target position in intron 10 of the CFTR gene;
wherein the target position is selected from the group consisting of position 1220, 2068, 3821, 4262, 5041, 5052, 5278, 5343, 5538, or 6150 of intron 10 in the CFTR gene.

35-44. (canceled)

45. A nucleic acid, comprising:
(a) a first nucleotide sequence encoding an RNA-guided DNA endonuclease, and
(b) a second nucleotide sequence encoding a guide RNA (gRNA), wherein the gRNA directs cleavage by the RNA-guided DNA endonuclease at a target position of a cystic fibrosis transmembrane regulator (CFTR) gene, wherein the target position is selected from the group consisting of position 1220, 2068, 3821, 4262, 5041, 5052, 5278, 5343, 5538, or 6150 of intron 10 in the CFTR gene;
wherein each of the first nucleotide sequence and the second nucleotide sequence is in operable linkage to a promoter.

46-52. (canceled)

53. A genetically edited lung cell or a precursor cell thereof, comprising a genetically edited endogenous cystic fibrosis transmembrane regulator (CFTR) gene, in which an exogenous nucleic acid is inserted into intron 10 of the endogenous CFTR gene, wherein the exogenous nucleic acid comprises a first nucleotide sequence encoding exons 11 to 27 of a CFTR gene.

54-58. (canceled)
